(19) United States
(12) Patent Application Publication
(10) Pub. No.: US 2004/0204577 A1
(43) Pub. Date: Oct. 14, 2004

(54) Novel peptide-forming enzyme gene

(75) Inventors: Seiichi Hara, Kawasaki (JP); Kenzo Yokozeki, Kawasaki (JP); Isao Abe, Kawasaki (JP); Naoto Tonouchi, Kawasaki (JP); Yasuko Jojima, Kawasaki (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Chuo-Ku (JP)

(21) Appl. No.: 10/763,179

(22) Filed: Jan. 26, 2004

Related U.S. Application Data
(60) Provisional application No. 60/491,612, filed on Aug. 1, 2003.

(30) Foreign Application Priority Data
Jan. 24, 2003 (JP) 2003-16765

(51) Int. Cl. C12P 21/06; C12N 9/48; C07H 21/04; C12N 1/21; C12N 15/74

(52) U.S. Cl. 536/23.2; 435/68.1; 435/69.1; 435/252.3; 435/320.1; 435/212

(57) ABSTRACT

DNA and recombinant DNA that encode a peptide-forming enzyme, a method for producing a peptide-forming enzyme, and a method for producing a dipeptide are disclosed. A method for producing a dipeptide includes producing a dipeptide from a carboxy component and an amine component by using a culture of a microbe belonging to the genus Sphingobacterium and having the ability to form the dipeptide from the carboxy component and the amine component, a microbial cell separated from the culture, treated microbial cell product of the microbe or a peptide-forming enzyme derived from the microbe.

### CROSS-REFERENCE TO RELATED APPLICATIONS

This application claims priority to Japanese patent application JP 2003-16765 filed on Jan. 24, 2003 and to U.S. provisional application U.S. No. 60/491,612 filed on Aug. 1, 2003. This application is also related to, but does not claim priority to, International Application PCT/JP03/09468 filed on Jul. 25, 2003 and Japanese patent application JP 2002-218957 filed on Jul. 26, 2002. The entire contents of each of the aforementioned applications is incorporated herein by reference.

### BACKGROUND OF THE INVENTION

1) Field of the Invention

The present invention relates to a novel enzyme that can form a peptide easily, at high yield and inexpensively without going through a complex synthetic method. More particularly, the present invention relates to a novel enzyme that catalyzes a peptide-forming reaction from a carboxy component and an amine component, to a microbe that produces the enzyme, and to a method for producing dipeptide using this enzyme or microbe.

2) Description of the Related Art

Peptides are used in the fields of pharmaceuticals, foods and various other fields. For example, since L-alanyl-L-glutamine has higher stability and water-solubility than L-glutamine, it is widely used as a component of fluid infusion and serum-free media.

Chemical synthesis methods, which have been known as methods for producing peptides, are not always easy. Known examples of such methods include a method that uses N-benzyloxycarbonylalanine (hereinafter, “Z-alanine”) and protected L-glutamine (see Bull. Chem. Soc. Jpn., 34, 739 (1961), Bull. Chem. Soc. Jpn., 35, 1966 (1962)), a method that uses Z-alanine and protected L-glutamic acid-γ-methyl ester (see Bull. Chem. Soc. Jpn., 37, 200 (1964)), a method that uses Z-alanine ester and unprotected glutamic acid (see Japanese Patent Application Laid-open Publication No. H1-96194), a method that involves synthesis of an N-(2-substituted)-propionyl glutamine derivative as an intermediate from a 2-substituted-propionyl halide as a raw material (see Patent Application Laid-open Publication No. H6-234715).

However, since all these methods require the introduction and elimination of protecting groups or the use of an optically active intermediate, they are not considered to be adequately satisfactory in terms of their industrial advantages.

On the other hand, widely known examples of typical peptide production methods using enzymes consist of a condensation reaction that uses an N-protected and C-unprotected carboxy component and an N-unprotected, C-protected amine component (hereinafter, “Reaction 1”), and a substitution reaction that uses an N-protected, C-protected carboxy component and an N-unprotected, C-protected amine component (hereinafter, “Reaction 2”). An example of Reaction 1 is a method for producing Z-aspartylphenylalanine methyl ester from Z-aspartic acid and phenylalanine methyl ester (see Japanese Patent Application Laid-open Publication No. S53-92729), while an example of Reaction 2 is a method for producing acetylphenylalanylleucine amide from acetylphenylalanine ethyl ester and leucine amide (see Biochemical J., 163, 531 (1977)). There have been reported very few research examples of method that uses an N-unprotected, C-protected carboxy component. An example of a substitution reaction that uses an N-unprotected, C-protected carboxy component and an N-unprotected, C-protected amine component (hereinafter, “Reaction 3”) is described in International Patent Publication WO 90/01555. For example, a method for producing arginylleucine amide from arginine ethyl ester and leucine amide may be mentioned of. Examples of substitution reactions that use an N-unprotected, C-protected carboxy component and an N-unprotected, C-unprotected amine component (hereinafter, “Reaction 4”) are described in European Patent Publication EP 278787A1 and European Patent Publication EP 359399B1. For example, a method for producing tyrosylalanine from tyrosine ethyl ester and alanine may be mentioned of.

### SUMMARY OF THE INVENTION

The most inexpensive production method among the aforementioned methods of Reactions 1 to 4 naturally falls within the class of Reaction 4, which involves the fewest protecting groups.

However, the example of Reaction 4 of the prior art (see European Patent Publication EP 278787A1) had the following major problems: (1) extremely slow rate of peptide production, (2) low peptide production yield, (3) the peptides that can be produced are limited to those that contain amino acids with comparatively high hydrophobicity, (4) the amount of enzyme added is extremely large, and (5) comparatively expensive carboxypeptidase preparations derived from molds, yeasts or plants are required. In the Reaction 4, there is no method known whatsoever that uses an enzyme derived from bacteria or yeasts other than the genus Saccharomyces, and there are no known method for producing alanylglutamine and other peptides that are highly hydrophilic. In consideration of this background, there is a need to develop an industrially inexpensive method for producing these peptides.

It is an object of the present invention to provide a novel enzyme that can form a peptide easily, at high yield and inexpensively without going through a complex synthesis method. More particularly, an object of the present invention is to provide a novel enzyme that catalyzes a peptide-forming reaction from a carboxy component and an amine component, a microbe that produces the enzyme, and a method for inexpensively producing a peptide using this enzyme or microbe.

As a result of conducting extensive research in consideration of the above object, the inventors of the present invention have found a novel enzyme that efficiently forms a peptide from newly discovered bacteria belonging to the genus Empedobacter, etc. and determined the sequence of this enzyme gene, thereby leading to completion of the present invention.

Namely, the present invention is as described below.

[1] A DNA encoding a protein (A) or (B):

(A) a protein having an amino acid sequence consisting of amino acid residues numbers 23 to 616 of an amino acid sequence described in SEQ ID NO: 6 of the Sequence Listing,

(B) a protein having an amino acid sequence including substitution, deletion, insertion, addition, and/or inversion of one or a plurality of amino acids in the amino acid sequence consisting of amino acid residues numbers 23 to 616 of the amino acid sequence described in SEQ ID NO: 6 of the Sequence Listing, and having peptide-forming activity.

[2] A DNA encoding a protein (C) or (D):

(C) a protein having an amino acid sequence consisting of amino acid residues numbers 21 to 619 of an amino acid sequence described in SEQ ID NO: 12 of the Sequence Listing,

(D) a protein that has an amino acid sequence including substitution, deletion, insertion, addition, and/or inversion of one or a plurality of amino acids in the amino acid sequence consisting of amino acid residues numbers 21 to 619 of the amino acid sequence described in SEQ ID NO: 12 of the Sequence Listing, and having peptide-forming activity.

[3] A DNA encoding a protein (E) or (F):

(E) a protein having an amino acid sequence consisting of amino acid residues numbers 23 to 625 of an amino acid sequence described in SEQ ID NO: 18 of the Sequence Listing,

(F) a protein having an amino acid sequence including substitution, deletion, insertion, addition, and/or inversion of one or a plurality of amino acids in the amino acid sequence consisting of amino acid residues numbers 23 to 625 of the amino acid sequence described in SEQ ID NO: 18 of the Sequence Listing, and having peptide-forming activity.

[4] A DNA encoding a protein (G) or (H):

(G) a protein having an amino acid sequence consisting of amino acid residues numbers 23 to 645 of an amino acid sequence described in SEQ ID NO: 23 of the Sequence Listing,

(H) a protein that has an amino acid sequence including substitution, deletion, insertion, addition, and/or inversion of one or a plurality of amino acids in the amino acid sequence consisting of amino acid residues numbers 23 to 645 of the amino acid sequence described in SEQ ID NO: 23 of the Sequence Listing, and having peptide-forming activity.

[5] A DNA encoding a protein (I) or (J):

(I) a protein having an amino acid sequence consisting of amino acid residues numbers 26 to 620 of an amino acid sequence described in SEQ ID NO: 25 of the Sequence Listing,

(J) a protein having an amino acid sequence including substitution, deletion, insertion, addition, and/or inversion of one or a plurality of amino acids in the amino acid sequence consisting of amino acid residues numbers 26 to 620 of an amino acid sequence described in SEQ ID NO: 25 of the Sequence Listing, and having peptide-forming activity.

[6] A DNA encoding a protein (K) or (L):

(K) a protein having an amino acid sequence consisting of amino acid residues numbers 18 to 644 of an amino acid sequence described in SEQ ID NO: 27 of the Sequence Listing,

(L) a protein having an amino acid sequence including substitution, deletion, insertion, addition, and/or inversion of one or a plurality of amino acids in the amino acid sequence consisting of amino acid residues numbers 18 to 644 of the amino acid sequence described in SEQ ID NO: 27 of the Sequence Listing, and having peptide-forming activity.

[7] A DNA encoding a protein (M) or (N):

(M) a protein that has an amino acid sequence described in SEQ ID NO: 6 of the Sequence Listing,

(N) a protein containing a mature protein region, having an amino acid sequence including substitution, deletion, insertion, addition, and/or inversion of one or a plurality of amino acids in the amino acid sequence described in SEQ ID NO: 6 of the Sequence Listing, and having peptide-forming activity.

[8] A DNA encoding a protein (O) or (P):

(O) a protein having an amino acid sequence described in SEQ ID NO: 12 of the Sequence Listing,

(P) a protein containing a mature protein region, having an amino acid sequence including substitution, deletion, insertion, addition, and/or inversion of one or a plurality of amino acids in the amino acid sequence described in SEQ ID NO: 12 of the Sequence Listing, and having peptide-forming activity.

[9] A DNA encoding a protein (Q) or (R):

(Q) a protein having an amino acid sequence described in SEQ ID NO: 18 of the Sequence Listing,

(R) a protein containing a mature protein region, having an amino acid sequence including substitution, deletion, insertion, addition, and/or inversion of one or a plurality of amino acids in the amino acid sequence described in SEQ ID NO: 18 of the Sequence Listing, and having peptide-forming activity.

[10] A DNA encoding a protein (S) or (T):

(S) a protein having an amino acid sequence described in SEQ ID NO: 23 of the Sequence Listing,

(T) a protein containing a mature protein region, having an amino acid sequence including substitution, deletion, insertion, addition, and/or inversion of one or a plurality of amino acids in the amino acid sequence described in SEQ ID NO: 23 of the Sequence Listing, and having peptide-forming activity.

[11] A DNA encoding a protein (U) or (V):

(U) a protein having an amino acid sequence described in SEQ ID NO: 25 of the Sequence Listing,

(V) a protein containing a mature protein region, having an amino acid sequence including substitution, deletion, insertion, addition, and/or inversion of one or a plurality of amino acids in the amino acid sequence described in SEQ ID NO: 25 of the Sequence Listing, and having peptide-forming activity.

[12] A DNA encoding a protein (W) or (X):

(W) a protein having an amino acid sequence described in SEQ ID NO: 27 of the Sequence Listing,

(X) a protein containing a mature protein region, having an amino acid sequence including substitution, deletion, insertion, addition, and/or inversion of one or a plurality of amino acids in the amino acid sequence described in SEQ ID NO: 27 of the Sequence Listing, and having peptide-forming activity.

[13] A DNA (a) or (b):

(a) a DNA having a base sequence consisting of bases numbers 127 to 1908 of a base sequence described in SEQ ID NO: 5 of the Sequence Listing,

(b) a DNA that hybridizes under stringent conditions with a DNA having a base sequence complementary to the base sequence consisting of bases numbers 127 to 1908 of the base sequence described in SEQ ID NO: 5 of the Sequence Listing, and encodes a protein that has peptide-forming activity.

[14] A DNA (c) or (d):

(c) a DNA having a base sequence consisting of bases numbers 121 to 1917 of a base sequence described in SEQ ID NO: 11 of the Sequence Listing,

(d) a DNA that hybridizes under stringent conditions with a DNA having a base sequence complementary to the base sequence consisting of bases numbers 121 to 1917 of the base sequence described in SEQ ID NO: 11 of the Sequence Listing, and encodes a protein that has peptide-forming activity.

[15] A DNA (e) or (f):

(e) a DNA having a base sequence consisting of bases numbers 127 to 1935 of a base sequence described in SEQ ID NO: 17 of the Sequence Listing,

(f) a DNA that hybridizes under stringent conditions with a DNA having a base sequence complementary to the base sequence consisting of bases numbers 127 to 1935 of the base sequence described in SEQ ID NO: 17 of the Sequence Listing, and encodes a protein that has peptide-forming activity.

[16] A DNA (g) or (h):

(g) a DNA having a base sequence consisting of bases numbers 127 to 1995 of a base sequence described in SEQ ID NO: 22 of the Sequence Listing,

(h) a DNA that hybridizes under stringent conditions with a DNA having a base sequence complementary to the base sequence consisting of bases numbers 127 to 1995 of the base sequence described in SEQ ID NO: 22 of the Sequence Listing, and encodes a protein that has peptide-forming activity.

[17] A DNA (i) or (j):

(i) a DNA having a base sequence consisting of bases numbers 104 to 1888 of the base sequence described in SEQ ID NO: 24 of the Sequence Listing,

(j) a DNA that hybridizes under stringent conditions with a DNA having a base sequence complementary to the base sequence consisting of bases numbers 104 to 1888 of the base sequence described in SEQ ID NO: 24 of the Sequence Listing, and encodes a protein that has peptide-forming activity.

[18] A DNA (k) or (l):

(k) a DNA having a base sequence consisting of bases numbers 112 to 1992 of a base sequence described in SEQ ID NO: 26 of the Sequence Listing,

(l) a DNA that hybridizes under stringent conditions with a DNA having a base sequence complementary to the base sequence consisting of bases numbers 112 to 1992 of the base sequence described in SEQ ID NO: 26 of the Sequence Listing, and encodes a protein that has peptide-forming activity.

[19] A DNA (m) or (n):

(m) a DNA having a base sequence consisting of bases numbers 61 to 1908 of a base sequence described in SEQ ID NO: 5 of the Sequence Listing,

(n) a DNA that hybridizes under stringent conditions with a DNA having a base sequence complementary to the base sequence consisting of bases numbers 61 to 1908 of the base sequence described in SEQ ID NO: 5 of the Sequence Listing, and encodes a protein that contains a mature protein region and has peptide-forming activity.

[20] A DNA (o) or (p):

(o) a DNA having a base sequence consisting of bases numbers 61 to 1917 of the base sequence described in SEQ ID NO: 11 of the Sequence Listing,

(p) a DNA that hybridizes under stringent conditions with a DNA having a base sequence complementary to the base sequence consisting of bases numbers 61 to 1917 of the base sequence described in SEQ ID NO: 11 of the Sequence Listing, and encodes a protein that contains a mature protein region and has peptide-forming activity.

[21] A DNA (q) or (r):

(q) a DNA having a base sequence consisting of bases numbers 61 to 1935 of the base sequence described in SEQ ID NO: 17 of the Sequence Listing,

(r) a DNA that hybridizes under stringent conditions with a DNA having a base sequence complementary to the base sequence consisting of bases numbers 61 to 1935 of the base sequence described in SEQ ID NO: 17 of the Sequence Listing, and encodes a protein that contains a mature protein region and has peptide-forming activity.

[22] A DNA (s) or (t):

(s) a DNA having a base sequence consisting of bases numbers 61 to 1995 of the base sequence described in SEQ ID NO: 22 of the Sequence Listing,

(t) a DNA that hybridizes under stringent conditions with a DNA having a base sequence complementary to the base sequence consisting of bases numbers 61 to 1995 of the base sequence described in SEQ ID NO: 22 of the Sequence Listing, and encodes a protein that contains a mature protein region and has peptide-forming activity.

[23] A DNA (u) or (v):

(u) a DNA having a base sequence consisting of bases numbers 29 to 1888 of a base sequence described in SEQ ID NO: 24 of the Sequence Listing,

(v) a DNA that hybridizes under stringent conditions with a DNA having a base sequence complementary to the base sequence consisting of bases numbers 29 to 1888 of the base sequence described in SEQ ID NO: 24 of the Sequence Listing, and encodes a protein that contains a mature protein region and has peptide-forming activity.

[24] A DNA (w) or (x):

(w) a DNA having a base sequence consisting of bases numbers 61 to 1992 of a base sequence described in SEQ ID NO: 26 of the Sequence Listing,

(x) a DNA that hybridizes under stringent conditions with a DNA having a base sequence complementary to the base sequence consisting of bases numbers 61 to 1992 of the base sequence described in SEQ ID NO: 26 of the Sequence Listing, and encodes a protein that contains a mature protein region and has peptide-forming activity.

[25] The DNA according to any one of [13] to [24], wherein stringent conditions are conditions under which washing is carried out at 60° C. at a salt concentration equivalent to 1×SSC and 0.1% SDS.

[26] A recombinant DNA comprising the DNA according to any one of [1] to [24].

[27] A transformed cell comprising introduced therein the recombinant DNA according to [26].

[28] A method for producing a peptide-forming enzyme, comprising: culturing the transformed cell according to [27] in a medium, and allowing a peptide-forming enzyme to accumulate in the medium and/or transformed cell.

[29] A method for producing a dipeptide, comprising: culturing the transformed cell according to [28] in a medium to obtain a culture, and mixing the culture with a carboxy component and an amine component to synthesize the dipeptide.

[30] A method for producing a dipeptide, comprising: producing a dipeptide from a carboxy component and an amine component by using a culture of a microbe belonging to the genus Sphingobacterium and having the ability to form the dipeptide from the carboxy component and the amine component, a microbial cell separated from the culture, a treated microbial cell product of the microbe, or a peptide-forming enzyme derived from the microbe.

[31] A recombinant DNA comprising the DNA according to [25].

[32] A transformed cell comprising introduced therein the recombinant DNA according to [31].

[33] A method for producing a peptide-forming enzyme comprising: culturing the transformed cell according to [32] in a medium, and allowing peptide-forming enzyme to accumulate in the medium and/or transformed cell.

[34] A method for producing a dipeptide comprising: culturing the transformed cell according to [32] in a medium to obtain a culture, and mixing the culture with a carboxy component and an amine component to synthesize, the dipeptide.

Furthermore, the amino acid sequence described in SEQ ID NO: 6 is specified by the DNA described in SEQ ID NO: 5 of the Sequence Listing. The amino acid sequence described in SEQ ID NO: 12 is specified by the DNA described in SEQ ID NO: 11. The amino acid sequence described in SEQ ID NO: 18 is specified by the DNA described in SEQ ID NO: 17. The amino acid sequence described in SEQ ID NO: 23 is specified by the DNA described in SEQ ID NO: 22. The amino acid sequence described in SEQ ID NO: 25 is specified by the DNA described in SEQ ID NO: 24. The amino acid sequence described in SEQ ID NO: 27 is specified by the DNA described in SEQ ID NO: 26.

According to the present invention, a novel enzyme is provided that can produce a peptide easily, at high yield and inexpensively by reducing complex synthetic methods such as introduction and elimination of protecting groups. The use of the enzyme of the present invention enables efficient industrial production of a peptide.

The other objects, features and advantages of the present invention are specifically set forth in or will become apparent from the following detailed descriptions of the invention when read in conjunction with the accompanying drawings.

### BRIEF DESCRIPTION OF THE DRAWINGS

FIG. 1 is a graph illustrating the optimum pH of the enzyme of Empedobacter of the present invention;

FIG. 2 is a graph illustrating the optimum temperature of the enzyme of Empedobacter of the present invention;

FIG. 3 is a graph illustrating the time course of L-alanyl-L-glutamine from L-alanine production methyl ester and L-glutamine; and

FIG. 4 is a bar graph illustrating the amount of enzyme present in a cytoplasm fraction (Cy) and a periplasm fraction (Pe).

### DETAILED DESCRIPTION

Hereinafter, the novel dipeptide-forming enzyme gene of the present invention and the dipeptide-forming enzyme that is the product of that gene.

### (1) Microbes Harboring the DNA of the Present Invention

The DNA of the present invention encodes a protein having the ability to form a peptide from a carboxy component and an amine component. In the present specification, a carboxy component refers to a component that provides a carbonyl site (CO) in a peptide bond (—CONH—), while an amine component refers to a component that provides an amino site (NH) in a peptide bond. In addition, in the present specification, unless otherwise indicated specifically, the term “peptide” when used alone refers to a polymer having at least one peptide bond. In addition, in the present specification, “dipeptide” refers to a peptide having one peptide bond.

Examples of microbes harboring the DNA of the present invention include bacteria belonging to the genus Empedobacter, genus Sphingobacterium, genus Pedobacter, genus Taxeobacter, genus Cyclobacterium or genus Psycloserpens, while more specific examples thereof include Empedobacter brevis strain ATCC 14234 (strain FERM P-18545, strain FERM BP-8113), Sphingobacterium sp. strain FERM BP-8124, Pedobacter heparinus strain IFO 12017, Taxeobacter gelupurpurascens strain DSMZ 11116, Cyclobacterium marinum strain ATCC 25205 and Psycloserpens burtonensis strain ATCC 700359. Empedobacter brevis strain ATCC 14234 (strain FERM P-18545, strain FERM BP-8113), Sphingobacterium sp. strain FERM BP-8124, Pedobacter heparinus strain IFO 12017, Taxeobacter gelupurpurascens strain DSMZ 11116, Cyclobacterium marinum strain ATCC 25205 and Psycloserpens burtonensis strain ATCC 700359 are microbes that were selected as a result of searching by the inventors of the present invention for microbes that produce an enzyme which forms a peptide from a carboxy component and an amine component at high yield.

Among the aforementioned strains of microbes, those microbes described with FERM numbers have been deposited at the independent administrative corporation, National Institute of Advanced Industrial Science and Technology, International Patent Organism Depository (Chuo Dai-6, 1-1 Higashi 1-Chome, Tsukuba-shi, Ibaraki-ken, Japan), and can be furnished by referring to each number.

Among the aforementioned strains of microbes, those microbes described with ATCC numbers have been deposited at the American Type Culture Collection (P.O. Box 1549, Manassas, Va. 20110, the United States of America), and can be furnished by referring to each number.

Among the aforementioned strains of microbes, those microbes described with IFO numbers have been deposited at the Institute of Fermentation, Osaka (2-17-85 Jusanbon-cho, Yodogawa-ku, Osaka-shi, Japan), and can be furnished by referring to each number.

Among the aforementioned strains of microbes, those microbes described with NBRC numbers have been deposited at the NITE Biological Resource Center of the National Institute of Technology and Evaluation (5-8 Kazusa-Kamaashi 2-Chome, Kisarazu-shi, Chiba-ken, Japan), and can be furnished by referring to each number.

Among the aforementioned strains of microbes, those microbes described with DSMZ numbers have been deposited at the Deutche Sammlung von Mikroorganismen und Zellkulturen GmbH (German Collection of Microbes and Cell Cultures) (Mascheroder Weg 1b, 38124 Braunschweig, Germany), and can be furnished by referring to each number.

Empedobacter brevis strain ATCC 14234 (strain FERM P-1 8545, strain FERM BP-8113) was deposited at the International Patent Organism Depository of the independent administrative corporation, National Institute of Advanced Industrial Science and Technology (Chuo Dai-6, 1-1 Higashi 1-Chome, Tsukuba-shi, Ibaraki-ken, Japan) on Oct. 1, 2001 and assigned the deposit number of FERM P-18545. Control of this organism was subsequently transferred to deposition under the provisions of the Budapest Treaty at the International Patent Organism Depository of the independent administrative corporation, National Institute of Advanced Industrial Science and Technology on Jul. 8, 2002 and was assigned the deposit number of FERM BP-8113 (indication of microbe: Empedobacter brevis strain AJ 13933).

Sphingobacterium sp. strain AJ 110003 was deposited at the International Patent Organism Depository of the independent administrative corporation, National Institute of Advanced Industrial Science and Technology on Jul. 22, 2002, and was assigned the deposit number of FERM BP-8124. Note that the strain AJ 110003 (FERM BP-8124) was identified to be the aforementioned Sphingobacterium sp. by the identification experiment described below. The strain FERM BP-8124 is a Gram-negative rod (0.7 to 0.8×1.5 to 2.0 μm) that forms no spore and is not motile. Its colonies are round with a completely smooth border, contain low protrusions and have a glossy, light yellow color. The organism grows at 30° C. and is catalase positive, oxidase positive and negative for the OF test (glucose), and was identified as a bacteria bacterium belonging to the genus Sphingobacterium based on these properties. Moreover, because of the properties that it is negative for nitrate reduction, negative for indole production, negative for acid production from glucose, arginine dihydrolase negative, urease positive, esculin hydrolysis positive, gelatin hydrolysis negative, β-galactosidase positive, glucose assimilation positive, L-arabinose assimilation negative, D-mannose assimilation positive, D-mannitol assimilation negative, N-acetyl-D-glucosamine assimilation positive, maltose assimilation positive, potassium gluconate assimilation negative, n-capric acid assimilation negative, adipic acid assimilation negative, dl-malic acid assimilation negative, sodium citrate assimilation negative, phenyl acetate assimilation negative and cytochrome oxidase positive, it was determined to have properties that are similar to those of Sphingobacterium multivorum or Sphingobacterium spiritivorum. Moreover, although results of analyses on the homology of the base sequence of the 16S rRNA gene indicate the highest degree of homology with Sphingobactetrium multivorum (98.8%), there was no strain with which the bacterial strain matched completely. Accordingly, this bacterial strain was therefore identified as Sphingobacterium sp.

### (2) Microbe Culturing

In order to obtain microbial cells of microbes having the DNA of the present invention, the microbes can be cultured and grown in a suitable medium. There is no particular restriction on the medium used for this purpose so far as it allows the microbes to grow. This medium may be an ordinary medium containing ordinary carbon sources, nitrogen sources, phosphorus sources, sulfur sources, inorganic ions, and organic nutrient sources as necessary.

For example, any carbon source may be used so far as the microbes can utilize it. Specific examples of the carbon source that can be used include sugars such as glucose, fructose, maltose and amylose, alcohols such as sorbitol, ethanol and glycerol, organic acids such as fumaric acid, citric acid, acetic acid and propionic acid and their salts, hydrocarbons such as paraffin as well as mixtures thereof.

Examples of nitrogen sources that can be used include ammonium salts of inorganic acids such as ammonium sulfate and ammonium chloride, ammonium salts of organic acids such as ammonium fumarate and ammonium citrate, nitrates such as sodium nitrate and potassium nitrate, organic nitrogen compounds such as peptones, yeast extract, meat extract and corn steep liquor as well as mixtures thereof.

In addition, nutrient sources used in ordinary media, such as inorganic salts, trace metal salts and-vitamins, can also be suitably mixed and used.

There is no particular restriction on culturing conditions, and culturing can be carried out, for example, for about 12 to about 48 hours while properly controlling the pH and temperature within a pH range of 5 to 8 and a temperature range of 15 to 40° C., respectively, under aerobic conditions.

Enzyme Purification

The DNA of the present invention encodes a peptide-forming enzyme. This peptide-forming enzyme can be purified from bacteria belonging to, for example, the genus Empedobacter. A method for isolating and purifying a peptide-forming enzyme from Empedobacter brevis is explained as an example of enzyme purification of the enzyme.

First, a microbial cell extract is prepared from the microbial cells of Empedobacter brevis, for example, the strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002) by disrupting the cells using a physical method such as ultrasonic crushing or an enzymatic method using a cell wall-dissolving enzyme and removing the insoluble fraction by centrifugation and so forth. The peptide-forming enzyme can then be purified by fractionating the microbial cell extract solution obtained in the above manner by combining ordinary protein purification methods such as anion exchange chromatography, cation exchange chromatography or gel filtration chromatography.

An example of a carrier for use in anion exchange chromatography is Q-Sepharose HP (manufactured by Amersham). The enzyme is recovered in the non-adsorbed fraction under conditions of pH 8.5 when the cell extract containing the enzyme is allowed to pass through a column packed with the carrier.

An example of a carrier for use in cation exchange chromatography is MonoS HR (manufactured by Amersham). After adsorbing the enzyme onto the column by allowing the cell extract containing the enzyme to pass through a column packed with the carrier and then washing the column, the enzyme is eluted with a buffer solution having a high salt concentration. At that time, the salt concentration may be sequentially increased or a concentration gradient may be applied. For example, in the case of using MonoS HR, the enzyme adsorbed onto the column is eluted with NaCl of about 0.2 to about 0.5 M.

The enzyme purified in the manner described above can then be further uniformly purified by gel filtration chromatography and so forth. An example of the carrier for use in gel filtration chromatography is Sephadex 200 pg (manufactured by Amersham).

In the aforementioned purification procedure, the fraction containing the enzyme can be verified by assaying the peptide-forming activity of each fraction according to the method indicated in the examples to be described later. The internal amino acid sequence of the enzyme purified in the manner described above is shown in SEQ ID NO: 1 and SEQ ID NO: 2 of the Sequence Listing.

### (4) DNA of the Present Invention and Transformants

(4-1) DNA of the Present Invention

A DNA of the present invention having the base sequence consisting of base numbers 61 to 1908 described in SEQ ID NO: 5 was isolated from Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002). The DNA consisting of bases numbers 61-1908 described in SEQ ID NO: 5 is a code sequence (hereinafter, “CDS”) portion. The base sequence consisting of bases numbers 61 to 1908 contains a signal sequence region and a mature protein region. The signal sequence region consists of bases numbers 61 to 126, while the mature protein region consists of bases . numbers 127 to 1908. Namely, the present invention provides both a peptide enzyme protein gene that contains a signal sequence, and a peptide enzyme protein gene in the form of a mature protein. The signal sequence contained in the sequence described in SEQ ID NO: 5 is a type of leader sequence, and the main function of the leader peptide encoded by this leader sequence is presumed to be excretion from inside the cell membrane to outside the cell membrane. The protein encoded by bases numbers 127 to 1908, namely the site excluding the leader peptide, is a mature protein, and is presumed to exhibit a high degree of peptide-forming activity.

The DNA having a base sequence consisting of bases numbers 61 to 1917 described in SEQ ID NO: 11, which is also a DNA of the present invention, was isolated from Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002). The DNA having a base sequence consisting of bases numbers 61 to 1917 is a code sequence (CDS) portion. The base sequence consisting of bases numbers 61 to 1917 contains a signal sequence region and a mature protein region. The signal sequence region is a region that consists of bases numbers 61 to 120, while the mature protein region is a region that consists of bases numbers 121 to 1917. Namely, the present invention provides both a gene for a peptide enzyme protein gene that contains a signal sequence, and a gene for a peptide enzyme protein gene in the form of a mature protein. The signal sequence contained in the sequence described in SEQ ID NO: 11 is a kind of leader sequence. The main function of a leader peptide encoded by the leader sequence is presumed to be excretion from inside the cell membrane to outside the cell membrane. The protein encoded by bases numbers 121 to 1917, namely the portion excluding the leader peptide, is a mature protein, and it is presumed to exhibit a high degree of peptide-forming activity.

A DNA of the present invention having the base sequence consisting of bases numbers 61 to 1935 described in SEQ ID NO: 17 was isolated from Pedobacter heparinus strain IFO 12017 (Depositary institution: Institute of Fermentation, Osaka, Address of depositary institution: 2-17-85 Jusanbon-cho, Yodogawa-ku, Osaka-shi, Japan). The DNA consisting of bases numbers 61 to 1935 described in SEQ ID NO: 17 is a CDS portion. A signal sequence region and a mature protein region are contained in the base sequence consisting of bases numbers 61 to 1935. The signal sequence region consists of bases numbers 61 to 126, while the mature protein region consists of bases numbers 127 to 1935. Namely, the present invention provides both a peptide enzyme protein gene that contains a signal sequence, and a peptide enzyme protein gene in the form of a mature protein. The signal sequence contained in the sequence described in SEQ ID NO: 17 is a type of leader sequence, and the main function of the leader peptide encoded by this leader sequence region is presumed to be excretion from inside the cell membrane to outside the cell membrane. The protein encoded by bases numbers 127 to 1935, namely the site excluding the leader peptide, is a mature protein, and is presumed to exhibit a high degree of peptide-forming activity.

A DNA of the present invention having a base sequence consisting of bases numbers 61 to 1995 described in SEQ ID NO: 22 was isolated from Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1b, 38124 Braunschweig, Germany). The DNA consisting of bases numbers 61 to 1995 described in SEQ ID NO: 22 is a CDS portion. A signal sequence region and a mature protein region are contained in the base sequence consisting of bases numbers 61 to 1995. The signal sequence region consists of bases numbers 61 to 126, while the mature protein region consists of bases numbers. 127 to 1995. Namely, the present invention provides both a peptide enzyme protein gene that contains a signal sequence, and a peptide enzyme protein gene in the form of a mature protein. The signal sequence contained in the sequence described in SEQ ID NO: 22 is a type of leader sequence, and the main function of the leader peptide encoded by this leader sequence region is presumed to be excretion from inside the cell membrane to outside the cell membrane. The protein encoded by bases numbers 127 to 1995, namely the site excluding the leader peptide, is a mature protein, and is presumed to exhibit a high degree of peptide-forming activity.

A DNA of the present invention having a base sequence consisting of bases numbers 29 to 1888 described in SEQ ID NO: 24 was isolated from Cyclobacterium marinum strain ATCC 25205 (Depositary institution: American Type Culture Collection, Address of depositary institution: P.O. Box 1549, Manassas, Va. 20110, the United States of America). The DNA consisting of bases numbers 29 to 1888 described in SEQ ID NO: 24 is a CDS portion. A signal sequence region and a mature protein region are contained in the base sequence consisting of bases numbers 29 to 1888. The signal sequence region consists of bases numbers 29 to 103, while the mature protein region consists of bases numbers 104 to 1888. Namely, the present invention provides both a peptide enzyme protein gene that contains a signal sequence, and a peptide enzyme protein gene in the form of a mature protein. The signal sequence contained in the sequence described in SEQ ID NO: 24 is a type of leader sequence, and the main function of the leader peptide encoded by this leader sequence region is presumed to be excretion from inside the cell membrane to outside the cell membrane. The protein encoded by bases numbers 104 to 1888, namely the site excluding the leader peptide, is a mature protein, and is presumed to exhibit a high degree of peptide-forming activity.

A DNA of the present invention having a base sequence consisting of bases numbers 61 to 1992 described in SEQ ID NO: 26 was isolated from Psycloserpens burtonensis strain ATCC 700359 (Depositary institution: American Type Culture Collection, Address of depositary institution: P.O. Box 1549, Manassas, Va. 20110, the United States of America). The DNA consisting of bases numbers 61 to 1992 described in SEQ ID NO: 26 is a CDS portion. A signal sequence region and a mature protein region are contained in the base sequence consisting of bases numbers 61 to 1992. The signal sequence region consists of bases numbers 61 to 111, while the mature protein region consists of bases numbers 112 to 1992. Namely, the present invention provides both a peptide enzyme protein gene that contains a signal sequence, and a peptide enzyme protein gene in the form of a mature protein. The signal sequence contained in the sequence described in SEQ ID NO: 26 is a type of leader sequence, and the main function of the leader peptide encoded by this leader sequence region is presumed to be excretion from inside the cell membrane to outside the cell membrane. The protein encoded by bases numbers 112 to 1992, namely the site excluding the leader peptide, is a mature protein, and is presumed to exhibit a high degree of peptide-forming activity.

Furthermore, the various gene recombination techniques described below can be carried out in compliance with the descriptions in publications such as Molecular Cloning, 2nd edition, Cold Spring Harbor Press (1989).

The DNA of the present invention can be acquired by polymerase chain reaction (hereinafter, “PCR”) (refer to PCR; White T. J. et al., Trends Genet., 5, 185 (1989)) or hybridization from a chromosomal DNA or a DNA library of Empedobacter brevis, Sphingobacterium sp., Pedobacter heparinus, Taxeobacter gelupurpurascens, Cyclobacterium marinum or Psycloserpens burtonensis. Primers for PCR can be designed based on the internal amino acid sequences determined based on peptide-forming enzyme purified as explained in the aforementioned section (3). In addition, since the base sequences of peptide-forming enzyme gene (SEQ ID NO: 5, SEQ ID NO: 11, SEQ ID NO: 22, SEQ ID NO: 24 and SEQ ID NO: 26) have been clearly determined by the present invention, primers or probes for hybridization can be designed on the basis of these base sequences, and the gene can also be isolated using a probe. If primers having sequences corresponding to the 5′-non-translated region and 3′-non-translated region are used as PCR primers, the entire length of the coding region of the present enzyme can be amplified. For example, in amplifying the region containing both the leader sequence and mature protein coding region described in SEQ ID NO: 5, specifically, an example of the 5′-side primer is a primer having the base sequence of the region upstream of base number 61 in SEQ ID NO: 5, while an example of the 3′-side primer is a primer having a sequence complementary to the base sequence of the region downstream of base number 1908.

Primers can be synthesized by the phosphoamidite method (see Tetrahedron Letters (1981), 22,1859) using, for example, the Model 380B DNA Synthesizer manufactured by Applied Biosystems in accordance with routine methods. The PCR reaction can be carried out, for example, in accordance with the method specified by the supplier such as the manufacturer using the Gene Amp PCR System 9600 (manufactured by Perkin-Elmer) and the Takara LA PCR In Vitro Cloning Kit (manufactured by Takara Shuzo).

Regardless of whether a leader sequence is contained or not, a DNA substantially identical to a DNA consisting of the CDS described in SEQ ID NO: 5 of the Sequence Listing is also included in the DNA of the present invention. Namely, a DNA substantially identical to the DNA of the present invention can be obtained by isolating a DNA that hybridizes under stringent conditions with a DNA having a base sequence complementary to the CDS described in SEQ ID NO: 5 of the Sequence Listing, or with a probe prepared from the same base sequence, and encodes a protein having peptide-forming activity, from DNAs encoding the present enzyme having a mutation or cells possessing that DNA.

Regardless of whether a leader sequence is contained or not, a DNA substantially identical to a DNA consisting of the CDS described in SEQ ID NO: 11 of the Sequence Listing is also included in the DNA of the present invention. Namely, a DNA substantially identical to the DNA of the present invention can be obtained by isolating a DNA that hybridizes, under stringent conditions, with a DNA having a base sequence complementary to the CDS described in SEQ ID NO: 11 of the Sequence Listing, or with a probe prepared from the same base sequence, and encodes a protein that has peptide-forming activity, from DNAs encoding the present enzyme having a mutation or cells possessing the DNA.

Regardless of whether a leader sequence is contained or not, a DNA substantially identical to a DNA consisting of the CDS described in SEQ ID NO: 17 of the Sequence Listing is also included in the DNA of the present invention. Namely, a DNA substantially identical to the DNA of the present invention can be obtained by isolating a DNA that hybridizes under stringent conditions with a DNA having a base sequence complementary to the CDS described in SEQ ID NO: 17 of the Sequence Listing, or with a probe prepared from the same base sequence, and encodes a protein having peptide-forming activity, from DNAs encoding the present enzyme having a mutation or cells possessing that DNA.

Regardless of whether a leader sequence is contained or not, a DNA substantially identical to a DNA consisting of the CDS described in SEQ ID NO: 22 of the Sequence Listing is also included in the DNA of the present invention. Namely, a DNA substantially identical to the DNA of the present invention can be obtained by isolating a DNA that hybridizes under stringent conditions with a DNA having a base sequence complementary to the CDS described in SEQ ID NO: 22 of the Sequence Listing, or with a probe prepared from the same base sequence, and encodes a protein having peptide-forming activity, from DNAs encoding the present enzyme having a mutation or cells possessing that DNA.

Regardless of whether a leader sequence is contained or not, a DNA substantially identical to a DNA consisting of the CDS described in SEQ ID NO: 24 of the Sequence Listing is also included in the DNA of the present invention. Namely, a DNA substantially identical to the DNA of the present invention can be obtained by isolating a DNA that hybridizes under stringent conditions with a DNA having a base sequence complementary to the CDS described in SEQ ID NO: 24 of the Sequence Listing, or with a probe prepared from the same base sequence, and encodes a protein having peptide-forming activity, from DNAs encoding the present enzyme having a mutation or cells possessing that DNA.

Regardless of whether a leader sequence is contained or not, a DNA substantially identical to a DNA consisting of the CDS described in SEQ ID NO: 26 of the Sequence Listing is also included in the DNA of the present invention. Namely, a DNA substantially identical to the DNA of the present invention can be obtained by isolating a DNA that hybridizes under stringent conditions with a DNA having a base sequence complementary to the CDS described in SEQ ID NO: 26 of the Sequence Listing, or with a probe prepared from the same base sequence, and encodes a protein having peptide-forming activity, from DNAs encoding the present enzyme having a mutation or cells possessing that DNA.

A probe can be produced, for example, in accordance with established methods based on, for example, the base sequence described in SEQ ID NO: 5 of the Sequence Listing. In addition, a method for isolating a target DNA by using a probe to find a DNA that hybridizes with the probe may also be carried out in accordance with established methods. For example, a DNA probe can be produced by amplifying a base sequence cloned in a plasmid or phage vector, cleaving the base sequence desired to be used as a probe with a restriction enzyme and then extracting the desired base sequence. The portion to be cleaved out can be adjusted depending on the target DNA.

The term “under a stringent conditions” as used herein refers to conditions under which a so-called specific hybrid is formed but no non-specific hybrid is formed. It is difficult to precisely express the conditions in numerical values. For example, mention may be made of a condition under which DNAs having high homologies, for example, 50% or more, preferably 80% or more, more preferably 90% or more, hybridize with each other and DNAs having lower homologies than these do not hybridize with each other, or ordinary conditions for rinse in Southern hybridization, i.e., 60° C. and a salt concentration corresponding to 1×SSC and 0.1% SDS, preferably 0.1×SSC and 0.1% SDS. Although the genes that hybridize under such conditions include those genes in which stop codons have occurred at certain locations along their sequences or which have lost activity due to a mutation in the active center, these can be easily removed by ligating them to a commercially available expression vector, expressing them in a suitable host, and assaying the enzyme activity of the expression product using a method to be described later.

However, in the case of a base sequence that hybridizes under stringent conditions as described above, it is preferable that the protein encoded by that base sequence retains about a half or more, preferably 80% or more, and more preferably 90% or more, of the enzyme activity of the protein having the amino acid sequence encoded by the original base sequence serving as the base be retained under conditions of 50° C. and pH 8. For example, when explained for on the case of, for example, a base sequence that hybridizes under stringent conditions with a DNA that has a base sequence complementary to the base sequence consisting of bases numbers 127 to 1908 of the base sequence described in SEQ ID NO: 5, it is preferable that the protein encoded by that base sequence retains about a half or more, preferably 80% or more, and more preferably 90% or more, of the enzyme activity of the protein having an amino acid sequence that consists of amino acid residues numbers 23 to 616 of the amino acid sequence described in SEQ ID NO: 6 under conditions of 50° C. and pH 8.

An amino acid sequence encoded by the CDS described in SEQ ID NO: 5 of the Sequence Listing is shown in SEQ ID NO: 6 of the Sequence Listing. An amino acid sequence encoded by the CDS described in SEQ ID NO: 11 of the Sequence Listing is shown in SEQ ID NO: 12 of the Sequence Listing. An amino acid sequence encoded by the CDS described in SEQ ID NO.: 17 of the Sequence Listing is shown in SEQ ID NO: 18 of the Sequence Listing. An amino acid sequence encoded by the CDS described in SEQ ID NO: 22 of the Sequence Listing is shown in SEQ ID NO: 23 of the Sequence Listing. An amino acid sequence encoded by the CDS described in SEQ ID NO: 24 of the Sequence Listing is shown in SEQ ID NO: 25 of the Sequence Listing. An amino acid sequence encoded by the CDS described in SEQ ID NO: 26 of the Sequence Listing is shown in SEQ ID NO: 27 of the Sequence Listing.

The entire amino acid sequence described in SEQ ID NO: 6 contains a leader peptide and a mature protein region, with amino acid residues numbers 1 to 22 constituting the leader peptide, and amino acid residues numbers 23 to 616 constituting the mature protein region. In addition, the entire amino acid sequence described in SEQ ID NO: 11 includes a leader peptide and a mature protein region, with amino acid residues numbers 1 to 20 constituting the leader peptide, and amino acid residues numbers 21 to 619 constituting the mature protein region.

The entire amino acid sequence described in SEQ ID NO: 18 contains a leader peptide and a mature protein region, with amino acid residues numbers 1 to 22 constituting the leader peptide, and amino acid residues numbers 23 to 625 constituting the mature protein region.

The entire amino acid sequence described in SEQ ID NO: 23 contains a leader peptide and a mature protein region, with amino acid residues numbers 1 to 22 constituting the leader peptide, and amino acid residues numbers 23 to 645 constituting the mature protein region.

The entire amino acid sequence described in SEQ ID NO: 25 contains a leader peptide and a mature protein region, with amino acid residues numbers 1 to 25 constituting the leader peptide, and amino acid residues numbers 26 to 620 constituting the mature protein region.

The entire amino acid sequence described in SEQ ID NO: 27 contains a leader peptide and a mature protein region, with amino acid residues numbers 1 to 17 constituting the leader peptide, and amino acid residues numbers 18 to 644 constituting the mature protein region.

The protein encoded by the DNA of the present invention is a protein in which the mature protein has peptide-forming activity, and a DNA that encodes a protein substantially identical to a protein having the amino acid sequence described in SEQ ID NO: 6, SEQ ID NO: 12, SEQ ID NO: 18, SEQ ID NO: 23, SEQ ID NO: 25, or SEQ ID NO: 27 of the Sequence Listing, regardless of whether it contains a leader peptide or not, is also included in the DNA of the present invention. (Note that, base sequences are specified from amino acid sequences in accordance with the codes of the universal codons.) Namely, the present invention provides DNAs that encode proteins indicated in (A) to (X) below:

(A) a protein having an amino acid sequence consisting of amino acid residues numbers 23 to 616 of an amino acid sequence described in SEQ ID NO: 6 of the Sequence Listing,

(B) a protein having an amino acid sequence including substitution, deletion, insertion, addition, and/or inversion of one or a plurality of amino acids in amino acid residues numbers 23 to 616 of the amino acid sequence described in SEQ ID NO: 6 of the Sequence Listing, and having peptide-forming activity,

(C) a protein having the amino acid sequence consisting of amino acid residue numbers 21 to 619 of an amino acid sequence described in SEQ ID NO: 12 of the Sequence Listing,

(D) a protein having an amino acid sequence including substitution, deletion, insertion, addition, and/or inversion of one or a plurality of amino acids in amino acid residue numbers 21 to 619 of the amino acid sequence described in SEQ ID NO: 12 of the Sequence Listing, and having peptide-forming activity,

(E) a protein having an amino acid sequence consisting of amino acid residues numbers 23 to 625 of an amino acid sequence described in SEQ ID NO: 18 of the Sequence Listing,

(F) a protein having an amino acid sequence including substitution, deletion, insertion, addition, and/or inversion of one or a plurality of amino acids in amino acid residues numbers 23 to 625 of the amino acid sequence described in SEQ ID NO: 18 of the Sequence Listing, and having peptide-forming activity,

(G) a protein having an amino acid sequence consisting of amino acid residues numbers 23 to 645 of an amino acid sequence described in SEQ ID NO: 23 of the Sequence Listing,

(H) a protein having an amino acid sequence including substitution, deletion, insertion, addition, and/or inversion of one or a plurality of amino acids in amino acid residues numbers 23 to 645 of the amino acid sequence described in SEQ ID NO: 23 of the Sequence Listing, and having peptide-forming activity,

(I) a protein having an amino acid sequence consisting of amino acid residues numbers 26 to 620 of an amino acid sequence described in SEQ ID NO: 25 of the Sequence Listing,

(J) a protein having an amino acid sequence including substitution, deletion, insertion, addition, and/or inversion of one or a plurality of amino acids in amino acid residues numbers 26 to 620 of the amino acid sequence described in SEQ ID NO: 25 of the Sequence Listing, and having peptide-forming activity,

(K) a protein having an amino acid sequence consisting of amino acid residues numbers 18 to 644 of an amino acid sequence described in SEQ ID NO: 32 of the Sequence Listing,

(L) a protein having an amino acid sequence including substitution, deletion, insertion, addition, and/or inversion of one or a plurality of amino acids in amino acid residues numbers 18 to 644 of the amino acid sequence described in SEQ ID NO: 32 of the Sequence Listing, and having peptide-forming activity,

(M) a protein having an amino acid sequence described in SEQ ID NO: 6 of the Sequence Listing,

(N) a protein containing a mature protein region, having an amino acid sequence including substitution, deletion, insertion, addition, and/or inversion of one or a plurality of amino acids in the amino acid sequence described in SEQ ID NO: 6 of the Sequence Listing, and having peptide-forming activity,

(O) a protein having the amino acid sequence described in SEQ ID NO: 12 of the Sequence Listing,

(P) a protein containing a mature protein region, having an amino acid sequence including substitution, deletion, insertion, addition, and/or inversion of one or a plurality of amino acids in an amino acid sequence described in SEQ ID NO: 12 of the Sequence Listing, and having peptide-forming activity,

(Q) a protein having an amino acid sequence described in SEQ ID NO: 18 of the Sequence Listing,

(R) a protein containing a mature protein region, having an amino acid sequence including substitution, deletion, insertion, addition, and/or inversion of one or a plurality of amino acids in the amino acid sequence described in SEQ ID NO: 18 of the Sequence Listing, and having peptide-forming activity,

(S) a protein having an amino acid sequence described in SEQ ID NO: 23 of the Sequence Listing,

(T) a protein containing a mature protein region, having an amino acid sequence including substitution, deletion, insertion, addition, and/or inversion of one or a plurality of amino acids in the amino acid sequence described in SEQ ID NO: 23 of the Sequence Listing, and having peptide-forming activity,

(U) a protein having an amino acid sequence described in SEQ ID NO: 25 of the Sequence Listing,

(V) a protein containing a mature protein region, having an amino acid sequence including substitution, deletion, insertion, addition, and/or inversion of one or a plurality of amino acids in the amino acid sequence described in SEQ ID NO: 25 of the Sequence Listing, and having peptide-forming activity;

(W) a protein having an amino acid sequence described in SEQ ID NO: 27 of the Sequence Listing, and

(X) a protein containing a mature protein region, having an amino acid sequence in the amino acid sequence described in SEQ ID NO: 27 of the Sequence Listing, and having peptide-forming activity.

Here, although the meaning of the term “a plurality of” varies depending on the locations and types of the amino acid residues in the three-dimensional structure of the protein, it is within a range that does not significantly impair the three-dimensional structure and activity of the protein of the amino acid residues, and is specifically 2 to 50, preferably 2 to 30, and more preferably 2 to 10. However, in the case of amino acid sequences including substitution, deletion, insertion, addition, and/or inversion of one or a plurality of amino acids in amino acid sequences of the proteins of (B), (D), (F), (H), (J), (L), (N), (P), (R), (T), (V) or (X), it is preferable that the proteins retain about half or more, more preferably 80% or more, and even more preferably 90% or more of the enzyme activity of the proteins in the state where no mutation is included, under conditions of 50° C. and pH 8. For example, explanation will be made in the case of (B); in the case of the amino acid sequence (B) including substitution, deletion, insertion, addition, and/or inversion of one or a plurality of amino acids in the amino acid sequence described in SEQ ID NO: 6 of the Sequence Listing, it is preferable that this protein retains about half or more, more preferably 80% or more, and even more preferably 90% or more of the enzyme activity of the protein having the amino acid sequence described in SEQ ID NO: 6 of the Sequence Listing, under conditions of 50° C. and pH 8.

A mutation of an amino acid like that indicated in the aforementioned (B) and so forth is obtained by modifying the base sequence so that an amino acid of a specific site in the present enzyme gene is substituted, deleted, inserted or added by, for example, site-directed mutagenesis. In addition, a modified DNA that described above can also be acquired by mutagenesis treatment known in the art. Mutagenesis treatment refers to, for example, a method in which a DNA encoding the present enzyme is treated in vitro with hydroxylamine and so forth, as well as a method in which Escherichia bacteria that possess a DNA encoding the present enzyme are treated by a mutagen normally used in artificial mutagenesis, such as ultraviolet irradiation, N-methyl-N′-nitro-N-nitrosoguanidine (NTG) or nitrous acid.

In addition, naturally-occurring mutations such as differences attributable to a microbe species or strain are also included in the base substitution, deletion, insertion, addition and/or inversion described above. By expressing a DNA having such a mutation in suitable cells and investigating the enzyme activity of the expression product, a DNA can be obtained that encodes a protein substantially identical to the protein described in SEQ ID NO: 6 or 12 of the Sequence Listing.

(4-2) Preparation of Transformants and Production of Peptide-Forming Enzymes

Peptide-forming enzymes can be produced by introducing a DNA of the present invention into a suitable host and expressing the DNA in that host.

Examples of hosts for expressing a protein specified by a DNA of the present invention that can be used include various prokaryotic cells such as bacteria belonging to the genus Escherichia such as Escherichia coli, Empedobacter, Sphingobacterium, Flavobacterium and Bacillus such as Bacillus subtilis, as well as various eukaryotic cells such as Saccharomyces cerevisiae, Pichia stipitis and Aspergillus oryzae.

A recombinant DNA used to introduce a DNA into a host can be prepared by inserting the DNA to be introduced into a vector corresponding to the type of host in which the DNA is to be expressed, in such a form that the protein encoded by that DNA can be expressed. In the case where a promoter unique to a peptide-forming enzyme gene of Empedobacter brevis and so forth functions in the host cells, the promoter can be used as a promoter for expressing the DNA of the present invention. In addition, another promoter that acts on in the host cells may be ligated to the DNA of the present invention and the DNA may be expressed under the control of the promoter as necessary.

Examples of transformation methods for introducing a recombinant DNA into host cells include the method of D. M. Morrison (see Methods in Enzymology, 68, 326 (1979)) or the method in which DNA permeability is increased by treating receptor microbial cells with calcium chloride (see Mandel, H. and Higa, A., J. Mol. Biol., 53,159 (1970)).

In the case of mass production of a protein using recombinant DNA technology, conjugating the protein within a transformant that produces the protein to form an inclusion body of protein is also a preferable mode for carrying out the present invention. Advantages of this expression and production method include protection of the target protein from digestion by proteases present in the microbial cells, and simple and easy purification of the target protein by crushing the microbial cells, followed by centrifugation and so forth.

The inclusion bodies of protein obtained in this manner are solubilized with a protein denaturant and the solubilized protein is converted to a properly folded, physiologically active protein by going through an activity regeneration procedure that consists primarily of lysing the protein with a protein denaturant followed by removal of the denaturant. There are numerous examples of this, including regeneration of the activity of human interleukin-2 (see Japanese Patent Application Laid-open Publication No. S61-257931).

To obtain an active protein from inclusion bodies of protein, a series of operations including solubilization and activity regeneration are required, and the procedure is more complex than in the case of producing the active protein directly. However, in the case of producing a large volume of protein that has a detrimental effect on microbial growth in microbial cells, that effect can be suppressed by accumulating the proteins in the form of inclusion bodies of inactive protein in the microbial cells.

Examples of mass production methods for producing a large volume of target protein in the form of inclusion bodies include a method in which a target protein is expressed independently under the control of a powerful promoter, and a method in which a target protein is expressed in the form of a fused protein with a protein that is known to be expressed in a large volume.

Hereinafter, the present invention will be explained more specifically taking as an example of a method for producing transformed Escherichia coli and using the transformed microbe to produce a peptide-forming enzyme. Furthermore, in the case of producing a peptide-forming enzyme in a microbe such as Escherichia coli, a DNA may be incorporated that encodes a precursor protein containing a leader sequence or a DNA may be incorporated that consists only of a mature protein region that does not contain a leader sequence, and the DNA can be suitably selected for the protein encoding sequence depending on the production conditions, form, usage conditions and so forth of the enzyme to be produced.

Promoters normally used in the production of heterogeneous proteins in Escherichia coli can be used as promoters for expressing a DNA encoding a peptide-forming enzyme. Examples of such promoters include T7 promoter, lac promoter, trp promoter, trc promoter, tac promoter, lambda phage PR promoter, PL promoter and other powerful promoters. In addition, examples of vectors that can be used include pUC19, pUC18, pBR322, pHSG299, pHSG298, pHSG399, pHSG398, RSF1010, pMW119, pMW118, pMW219, and pMW218. Besides, vectors of phage DNA can also be used. Moreover, expression vectors can be used that contain promoters and are capable of expressing an inserted DNA sequence, including the promoter can be used.

In order to produce a peptide-forming enzyme in the form of an inclusion body of fused protein, a gene that encodes another protein, and preferably a hydrophilic peptide is ligated upstream or downstream of the peptide-forming enzyme gene to obtain a fused protein gene. The gene that encodes another protein in this manner may be any gene that increases the amount of the fused protein accumulated, and enhances the solubility of the fused protein after the denaturation and regeneration steps. Examples of candidates for such genes include T7 gene 10, β-galactosidase gene, dehydrofolate reductase gene, γ-interferon gene, interleukin-2 gene and prochymosin gene.

When these genes are ligated to a gene that encodes a peptide-forming enzymes, the both genes are ligated so that their reading frames of codons are consistent. The genes may be ligated at a proper restriction enzyme site or a synthetic DNA having a proper sequence may be utilized.

Further, to increase a production amount of the peptide-forming enzyme, it is preferable in some cases that a terminator, which is a transcription terminating sequence, be ligated to downstream of the fusion protein gene. The terminator includes, for example, a T7 terminator, an fd phage terminator, a T4 terminator, a tetracycline resistant gene terminator, and an Escherichia coli trpA gene terminator.

As the vectors for introducing a gene that encodes a peptide-forming enzyme or a fused protein between the peptide-forming enzyme and another protein in Escherichia coli are preferred so-called multi-copy type vectors, examples of which include a plasmid having a replication origin derived from ColE1, for example, a pUC-based plasmid, and a pBR322-based plasmid or derivatives thereof. The “derivatives” as used herein refer to those plasmids that are subjected to modification by substitution, deletion, insertion, addition and/or inversion of bases. Note that the modification as used herein includes modifications by a mutagenesis treatment with a mutagen or UV irradiation, or modifications by spontaneous mutation.

To screen transformants, it is preferable that the vectors have markers such as an ampicillin resistant gene. Such plasmids include commercially available expression vectors having potent promoters (a pUC-based vector (manufactured by Takara Shuzo, Co., Ltd.), pRROK-based vector (manufactured by Clonetech Laboratories, Inc.), pKK233-2 (manufactured by Clonetech Laboratories, Inc.) and so forth.

A recombinant DNA is obtained by ligating a DNA fragment to a vector DNA; in the DNA fragment, a promoter, a gene encoding L-amino acid amide hydrolase or a fused protein consisting of an L-amino acid amide hydrolase and another protein, and depending on the case, a terminator are ligated in that order.

When E. coli is transformed using the recombinant DNA and the resulting E. coli is cultured, a peptide-forming enzyme or a fused protein consisting of the peptide-forming enzyme and another protein is expressed and produced. Although a strain that is normally used in the expression of a heterogeneous gene can be used as a host to be transformed, Escherichia coli strain JM109, for example, is preferable. Methods for carrying out transformation and methods for screening out transformants are described in Molecular Cloning, 2nd Edition, Cold Spring Harbor Press (1989) and other publications.

In the case of expressing a peptide-forming enzyme in the form of a fusion protein, the peptide-forming enzyme may be cleaved out using a restriction protease that uses a sequence not present in the peptide-forming enzyme, such as blood coagulation factor Xa or kallikrein, as the recognition sequence.

A medium normally used for culturing E. coli, such as M9-casamino acid medium or LB medium, may be used for as the a production medium. In addition, culturing conditions and production induction conditions are suitably selected according to the marker of the vector used, promoter, type of host microbe and so forth.

The following method can be used to recover the peptide-forming enzyme or fused protein consisting of the peptide-forming enzyme and another protein. If the peptide-forming enzyme or its fused protein has been solubilized in the microbial cells, the microbial cells are recovered and then crushed or lysed so that they can be used as a crude enzyme liquid. Moreover, the peptide-forming enzyme or its fused protein can be purified prior to use by ordinary techniques such as precipitation, filtration or column chromatography as necessary. In this case, a purification method can also be used that uses an antibody of the peptide-forming enzyme or its fused protein.

In the case where inclusion bodies of protein are formed, the inclusion bodies are solubilized with a denaturant. Although they may be solubilized together with the microbial cell protein, it is preferable in consideration of the subsequent purification procedure that the inclusion bodies are taken out and then solubilized. Conventionally known methods may be used to recover the inclusion bodies from the microbial cells. For example, the inclusion bodies can be recovered by crushing the microbial cells followed by centrifugation. Examples of denaturants capable of solubilizing the inclusion bodies include quanidine guanidine hydrochloride (for example, 6 M, pH 5 to 8) and urea (for example, 8 M) and the like.

A protein that has activity is regenerated by removing these denaturants by dialysis or the like. A Tris-HCl buffer solution, a phosphate buffer solution or the like may be used as a dialysis solution to be used in dialysis, and its concentration may be, for example, 20 mM to 0.5 M, while its pH may be, for example, 5 to 8.

The protein concentration during the regeneration step is preferably held to about 500 μg/ml or less. The dialysis temperature is preferably 5° C. or lower to prevent the regenerated peptide-forming enzyme from undergoing self-crosslinking. Moreover, the method for removing the denaturants includes dilution or ultrafiltration in addition to dialysis, and it is expected the activity can be regenerated whichever denaturant is used.

### (5) Properties of Enzyme Encoded by DNA of the Present Invention

The activity of the enzyme encoded by the DNA of the present invention can be assayed by, for example, allowing the enzyme to react in a borate buffer solution containing an amino acid ester and an amine as substrates, and then quantifying the peptide formed. In a more concrete example, the reaction is carried out at 25° C. for several minutes using a borate buffer solution (pH 9.0) containing 100 mM L-alanine methyl ester and 200 mM L-glutamine.

The activity unit of the enzyme used in the present invention is defined such that 1 unit (U) is the amount of enzyme that produces 1 micromole (μmole) of peptide in 1 minute under the condition of reacting at 25° C. using a 100 mM borate buffer solution (pH 9.0) containing 100 mM L-alanine methyl ester and 200 mM L-glutamine.

A protein encoded by the DNA of the present invention is a peptide-forming enzyme protein. Peptide-forming activity refers to the activity that forms a peptide from a carboxy component and an amine component. Hereinafter, a preferable mode of the enzyme encoded by the DNA of the present invention will be explained on its properties.

One preferable mode of the enzyme encoded by the DNA of the present invention includes an enzyme that has the abilities described below, for which the dipeptide production rate is used as an indicator. Namely, one preferable mode of the enzyme of the present invention includes an enzyme that has the ability to form a peptide from a carboxy component and an amino component, and has a production rate of L-alanyl-L-glutamine in the dipeptide formation reaction under the conditions of (i) to (iv) below of preferably 0.03 mM/min or more, more preferably 0.3 mM/min or more, and particularly preferably 1.0 mM/min or more. The conditions of the dipeptide formation reaction are as follows:

(i) the carboxy component is L-alanine methyl ester hydrochloride (100 mM);

(ii) the amine component is L-glutamine (200 mM);

(iii) the pH is 9.0; and,

(iv) the amount of homogeneously purified enzyme added is less than 0.61 mg/ml as a protein amount.

The aforementioned production rate far exceeds the conventional production rate for peptide synthesis using an enzyme, and the enzyme of the present invention has the ability to catalyze peptide synthesis at an extremely rapid rate.

The aforementioned amount of enzyme added indicates a final amount of the enzyme that is added to the reaction system, and addition of the enzyme of 0.01 mg/ml or more, and preferably 0.02 mg/ml or more, as protein amount is desirable. The term “protein amount” refers to the value indicated by a calorimetric method with Coomassie brilliant blue using a protein assay CBB solution (manufactured by Nakarai) and bovine serum albumin as a standard substance.

In a specific example of the procedure for assaying the enzyme activity, the enzyme activity can be assayed by allowing the enzyme to react in a borate buffer solution containing an amino acid ester and an amine as substrates and quantifying the resulting peptide. In a more specific example, mention may be made of a method in which the enzyme is allowed to react for several minutes at 25° C. using a 100 mM borate buffer solution (pH 9.0) containing 100 mM L-alanine methyl ester and 200 mM L-glutamine.

In addition, a preferable mode of the enzyme encoded by the DNA of the present invention includes an enzyme having the property by which both an amino acid ester and an amino acid amide can be used as a substrate for the carboxy component. The words “both an amino acid ester and an amino acid amide can be used as a substrate” mean that at least one type or more of amino acid ester and at least one type or more of amino acid amide can be used as a substrate. In addition, one preferable mode of the enzyme of the present invention includes an enzyme that has the property by which all of an amino acid, a C-protected amino acid and an amine can be used as a substrate for the amine component. The words “an amino acid, a C-protected amino acid, and an amine can be used as a substrate” mean that at least one type or more of amino acid, at least one type or more of C-protected amino acid, and at least one type or more of amine can be used as a substrate. Having a wide range of substrate specificity with respect to the carboxy component or the amino component, the enzyme of the present invention is preferable in that a wide range of raw materials can be selected, which in turn is favorable in terms of cost and production equipment in the case of industrial production.

Specific examples of the carboxy component include L-amino acid esters, D-amino acid esters, L-amino acid amides and D-amino acid amides. In addition, the amino acid esters include not only amino acid esters corresponding to naturally-occurring amino acids, but also amino acid esters corresponding to non-naturally-occurring amino acids or their derivatives. Furthermore, examples of the amino acid esters include a-amino acid esters as well as β-, γ-, and ω-amino acid esters and the like, which have different amino group bonding sites. Typical examples of amino acid esters include methyl esters, ethyl esters, n-propyl esters, iso-propyl esters, n-butyl esters, iso-butyl esters, and tert-butyl esters of amino acids.

Specific examples of the amine component include L-amino acids, C-protected L-amino acids, D-amino acids, C-protected D-amino acids and amines. In addition, examples of the amines include not only naturally-occurring amines, but also non-naturally-occurring amines or their derivatives. In addition, examples of the amino acids include not only naturally-occurring amino acids, but also non-naturally-occurring amino acids or their derivatives. These include α-amino acids as well as β-, γ- and ω-amino acids and the like, which have different amino group bonding sites.

Further, in another aspect, one preferable mode of the enzyme encoded by the DNA of the present invention includes an enzyme in which the pH range over which the peptide-forming reaction can be catalyzed is 6.5 to 10.5. The ability of the enzyme of the present invention to catalyze this reaction over such a wide pH range as stated above is preferable in that it allows flexible accommodation of industrial production that could be subject to the occurrence of various restrictions. However, in the actual production of peptides, it is preferable to use the enzyme by further adjusting to an optimum pH corresponding to the acquired enzyme so as to maximize the catalytic performance of the enzyme.

Moreover, in another aspect, one preferable mode of the enzyme encoded by the DNA of the present invention includes an enzyme for which the temperature range over which the enzyme is capable of catalyzing the peptide-forming reaction is within the range of 0 to 60° C. Since the enzyme of the present invention is able to catalyze the reaction over a wide temperature range, it is preferable in that it allows flexible accommodation of industrial production that could be subject to the occurrence of various restrictions. However, in the actual production of peptides, it is preferable to use the enzyme by further adjusting to an optimum temperature corresponding to the acquired enzyme so as to maximize the catalytic performance of the enzyme.

### (6) Dipeptide Production Method

The method for producing dipeptide of the present invention includes reaction between a carboxy component and an amine component in the presence of a predetermined enzyme. The dipeptide production method of the present invention includes allowing an enzyme, or enzyme-containing substance, having the ability to form a peptide from a carboxy component and an amine component, to act on the carboxy component and the amine component to synthesize a dipeptide.

The method of allowing the enzyme or enzyme-containing substance used in the present invention to act on the carboxy component and the amine component may be mixing the enzyme or enzyme-containing substance, the carboxy component, and the amine component with each other. More specifically, a method of adding the enzyme or enzyme-containing substance to a solution containing a carboxy component and an amine component and allowing them to react may be used. Alternatively, in the case of using a microbe that produces that enzyme, a method may be used that includes culturing the microbe that forms that enzyme, producing and accumulating the enzyme in the microbe or microbial culture broth, and then adding the carboxy component and amine component to the culture broth. The produced dipeptide can then be collected by established methods and purified as necessary.

The term “enzyme-containing substance” means any substance so far as it contains the enzyme, and examples of specific forms thereof include a culture of microbes that produce the enzyme, microbial cells isolated from the culture, and a product obtained by treating the microbial cells (hereinafter, “treated microbial cell product”). A culture of microbes refers to what is obtained by culturing a microbe, and more specifically, to a mixture of microbial cells, the medium used for culturing the microbe, and substances produced by the cultured microbe, and so forth. In addition, the microbial cells may be washed and used in the form of washed microbial cells. In addition, the treated microbial cell product includes the products of crushed, lysed or freeze-dried microbial cells, and the like, and also includes a crude enzyme recovered by treating microbial cells, and so forth, as well as a purified enzyme obtained by purification of the crude enzyme, and so forth. A partially purified enzyme obtained by various types of purification methods may be used for the purified enzyme, or immobilized enzymes may be used that have been immobilized by a covalent bonding method, an adsorption method, an entrapment method, or the like. In addition, since some microbes are partially lysed during culturing depending on the microbes used, the culture supernatant may also be used as the enzyme-containing substance in such cases.

In addition, wild strains may be used as the microbes that contain the enzyme, or gene recombinant strains that express the enzyme may also be used. The microbes are not limited to enzyme microbial cells, but rather acetone-treated microbial cells, freeze-dried microbial cells or other treated microbial cells may also be used. Immobilized microbial cells and an immobilized treated microbial cell product obtained by immobilizing the microbial cells or treated microbial cell product by covalent bonding, adsorption, entrapment or other methods, as well as treated immobilized microbial cells, may also be used.

The term “homogeneously purified enzyme” as used herein refers to an enzyme that shows a homogeneous band in an electrophoresis experiment of a purified protein containing an enzyme protein and has enzyme activity of that enzyme.

Furthermore, when using cultures, cultured microbial cells, washed microbial cells or a treated microbial cell product that has been obtained by crushing or lysing microbial cells, it is often the case that an enzyme exists therein that decomposes the formed peptides without being involved in peptide formation. In this situation, it may be rather preferable in some cases to add a metal protease inhibitor like ethylene diamine tetraacetic acid (EDTA). The addition amount is within the range of 0.1 millimolar (mM) to 300 mM, and preferably 1 mM to 100 mM.

A preferable mode of the dipeptide production method of the present invention is a method in which the transformed cells described in the previously described section (4-2) are cultured in a medium, and a peptide-forming enzyme is allowed to accumulate in the medium and/or transformed cells. Since the peptide-forming enzyme can be easily produced in large volumes by using a transformant, dipeptides can be produced in large amounts and rapidly.

The amount of enzyme or enzyme-containing substance used may be enough if it is an amount at which the target effect is demonstrated (effective amount), and this effective amount can be easily determined through simple, preliminary experimentation by a person with ordinary skill in the art. In the case of using the enzyme, for example, the amount used is about 0.01 U to about 100 U, while in the case of using washed microbial cells, the amount used is about 0.1 g/L to about 500 g/L.

Any carboxy component may be used as far as it can form a peptide by condensation with the other substrate in the form of the amine component. Examples of carboxy component include L-amino acid esters, D-amino acid esters, L-amino acid amides and D-amino acid amides as well as organic acid esters not having an amino group. In addition, examples of amino acid esters include not only amino acid esters corresponding to naturally-occurring amino acids, but also amino acid esters corresponding to non-naturally-occurring amino acids or their derivatives. In addition, examples of amino acid esters include α-amino acid esters as well as β-, γ- and ω-amino acid esters and the like having different amino group bonding sites. Typical examples of amino acid esters include methyl esters, ethyl esters, n-propyl esters, iso-propyl esters, n-butyl esters, iso-butyl esters and tert-butyl esters of amino acids.

Any amine component may be used as far as it can form a peptide by condensation with the other substrate in the form of the carboxy component. Examples of the amine component include L-amino acids, C-protected L-amino acids, D-amino acids, C-protected D-amino acids and amines. In addition, examples of the amines include not only naturally-occurring amines, but also non-naturally-occurring amines or their derivatives. In addition, examples of the amino acids include not only naturally-occurring amino acids, but also non-naturally-occurring amino acids or their derivatives. These include α-amino acids as well as β-, γ- or ω-amino acids and the like having different amino group bonding sites.

The concentrations of the carboxy component and amine component serving as starting materials are 1 mM to 10 M, and preferably 0.05 M to 2 M, respectively; however, there are cases where it is preferable to add amine component in an amount equimolar or excess molar with respect to the carboxy component. In addition, in cases where high concentrations of substrates inhibit the reaction, these can be successively added during the reaction after they are adjusted to concentrations that do not cause inhibition.

The reaction temperature that allows synthesis of peptide is 0 to 60° C., and preferably 5 to 40° C. In addition, the reaction pH that allows synthesis of peptide is 6.5 to 10.5, and preferably 7.0 to 10.0.

### EXAMPLES

Hereinafter, the present invention will be explained by examples. However, the present invention is not limited to these examples. In addition to confirmation by ninhydrin coloring of thin-film chromatograms (qualitative), quantitative determinations were made by the following high-performance liquid chromatography in order to assay products. Column: InertsiL ODS-2 (manufactured by GL Science, Inc.), eluate: an aqueous phosphate solution containing 5.0 mM sodium 1-octanesulfonate (pH 2.1):methanol=100:15 to 50, flow rate: 1.0 mL/min, detection: 210 nanometers (hereinafter, “nm”).

### Example 1

### Microbe Culturing (Empedobacter brevis Strain FERM BP-8113)

A 50 mL medium (pH 6.2) containing 5 grams (g) of glucose, 5 g of ammonium sulfate, 1 g of monopotassium phosphate, 3 g of dipotassium phosphate, 0.5 g of magnesium sulfate, 10 g of yeast extract and 10 g of peptone in 1 liter (L) was transferred to a 500 mL Sakaguchi flask and sterilized at 115° C. for 15 minutes. This medium was then inoculated with one loopful cells of Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002) that had been cultured at 30° C. for 16 hours in the same medium, followed by shake culturing at 30° C. for 16 hours and 120 strokes/min.

### Example 2

### Production of Peptide Using Microbial Cells

Microbial cells were collected by centrifuging (10,000 rounds per minute (rpm), 15 minutes) the culture broth obtained in Example 1, followed by suspending them to a concentration of 100 g/L in 100 mM borate buffer (pH 9.0) containing 10 mM EDTA. After respectively adding 1 mL of the suspension to 1 mL each of 100 mM borate buffer solutions (pH 9.0) containing 10 mM EDTA, 200 mM of the following carboxy components, and 400 mM of the following amino acids to make a final volume of 2 mL, the reaction was carried out at 18° C. for 2 hours. The peptides that were formed as a result of this reaction are shown in Table 1.

TABLE 1

| Carboxy | Amine | Formed | | Carboxy | Amine | Formed | |
|---|---|---|---|---|---|---|---|
| component | component | peptide | (mM) | component | component | peptide | (mM) |
| L-Ala-OMe | L-Leu | L-Ala-L-Leu | 38.2 | Gly-OMe | L-His | L-Gly-L-His | 22.1 |
| | L-Met | L-Ala-L-Met | 68.3 | L-Ser-OMe | L-Ser | L-Ser-L-Ser | 29.0 |
| | L-Phe | L-Ala-L-Phe | 62.4 | L-Val-OMe | L-Met | L-Val-L-Met | 10.5 |
| | L-Ser | L-Ala-L-Ser | 51.3 | L-Met-OMe | L-Phe | L-Met-L-Phe | 28.5 |
| | L-His | L-Ala-L-His | 52.1 | L-Thr-OMe | L-Leu | L-Thr-L-Leu | 23.0 |
| | L-Arg | L-Ala-L-Arg | 72.1 | L-Ile-OMe | L-Met | L-Ile-L-Met | 8.3 |
| | L-Gln | L-Ala-L-Gln | 68.0 | | | | |

Hydrochloride salts were used for all the carboxy components.

### Example 3

### Enzyme Purification

The procedure after centrifugation was carried out either on ice or at 4° C. Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002) was cultured in the same manner in as Example 1, and the microbial cells were collected by centrifugation (10,000 rpm, 15 minutes). After washing 16 g of microbial cells with 50 mM Tris-HCl buffer (pH 8.0), they were suspended in 40 milliliters (ml or mL) of the same buffer and subjected to ultrasonic crushing treatment for 45 minutes at 195 watts. This ultrasonic crushed suspension was then centrifuged (10,000 rpm, 30 minutes) to remove the crushed cell fragments and obtain a supernatant. This supernatant was dialyzed overnight against 50 mM Tris-HCl buffer (pH 8.0) followed by removal of the insoluble fraction by ultracentrifugation (50,000 rpm, 30 minutes) to obtain a soluble fraction in the form of the supernatant liquid. The resulting soluble fraction was applied to a Q-Sepharose HP column (manufactured by Amersham) pre-equilibrated with Tris-HCl buffer (pH 8.0), and the active fraction was collected from the non-adsorbed fraction. This active fraction was dialyzed overnight against 50 mM acetate buffer (pH 4.5) followed by removal of the insoluble fraction by centrifugation (10,000 rpm, 30 minutes) to obtain a dialyzed fraction in the form of the supernatant liquid. This dialyzed fraction was then applied to a Mono S column (manufactured by Amersham) pre-equilibrated with 50 mM acetate buffer (pH 4.5) to elute enzyme at a linear concentration gradient of the same buffer containing 0 to 1 M NaCl. The fraction that had the lowest level of contaminating protein among the active fractions was applied to a Superdex 200 pg column (manufactured by Amersham) pre-equilibrated with 50 mM acetate buffer (pH 4.5) containing 1 M NaCl, and gel filtration was performed by allowing the same buffer (pH 4.5) containing 1 M NaCl to flow through the column to obtain an active fraction solution. As a result of performing these operations, the peptide-forming enzyme used in the present invention was confirmed to have been uniformly purified based on the experimental results of electrophoresis. The enzyme recovery rate in the aforementioned purification process was 12.2% and the degree of purification was 707 times.

### Example 4

### Measurement of Enzyme Molecular Weight

SDS-Gel Electrophoresis

A 0.3 microgram (μg) equivalent of the purified enzyme fraction obtained by the method of Example 3 was applied to polyacrylamide electrophoresis. 0.3% (w/v) Tris, 1.44% (w/v) glycine and 0.1% (w/v) sodium laurylsulfate were used for the electrophoresis buffer solution, a gel having a concentration gradient of a gel concentration of 10 to 20% (Multigel 10 to 20, manufactured by Daiichi Pure Chemicals) was used for the polyacrylamide gel, and Pharmacia molecular weight markers were used as the molecular weight markers. Following completion of electrophoresis, the gel was stained with Coomassie brilliant blue R-250, and a uniform band was detected at the location of a molecular weight of about 75 kilodaltons (kDa).

Gel Filtration

The purified enzyme fraction obtained by the method of Example 3 was applied to a Superdex 200 pg column (manufactured by Amersham) pre-equilibrated with 50 mM acetate buffer (pH 4.5) containing 1 M NaCl, and gel filtration was carried out by allowing the same buffer (pH 4.5) containing 1 M NaCl to flow through the column to measure the molecular weight. Pharmacia molecular weight markers were used as standard proteins having known molecular weights to prepare a calibration curve. As a result, the molecular weight of the enzyme was about 150 kDa.

Based on the results of SDS-gel electrophoresis and gel filtration, the enzyme was suggested to be a homodimer having a molecular weight of about 75 kDa.

### Example 5

### Enzyme Optimum pH

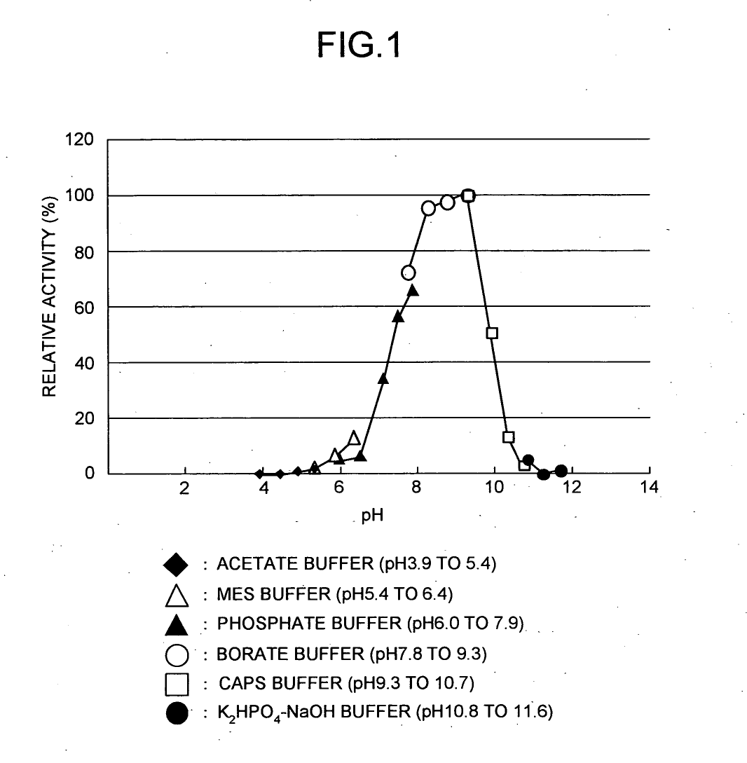

The effects of pH were examined in the reaction in which L-alanyl-L-glutamine is formed from L-alanine methyl ester hydrochloride and L-glutamine. Acetate buffer (pH 3.9 to 5.4), MES buffer (pH 5.4 to 6.4), phosphate buffer (pH 6.0 to 7.9), borate buffer (pH 7.8 to 9.3), CAPS buffer (pH 9.3 to 10.7), and K₂HPO₄—NaOH buffer (pH 10.8 to 11.6) were used as buffers. 1 microliter (II) of the Mono S fraction enzyme obtained in Example 3 (about 180 U/ml) was added to 100 μl of each buffer at 100 mM containing 100 mM L-alanine methyl ester, 200 mM L-glutamine and 10 mM EDTA and allowed to react at 18° C. for 5 minutes to measure the effects of pH on the reaction. The results expressed by assigning a value of 100% to the case of using borate buffer (pH 9.3) are shown in FIG. 1. As a result, the optimum enzyme pH was found to be 8 to 9.5.

### Example 6

### Enzyme Optimum Temperature

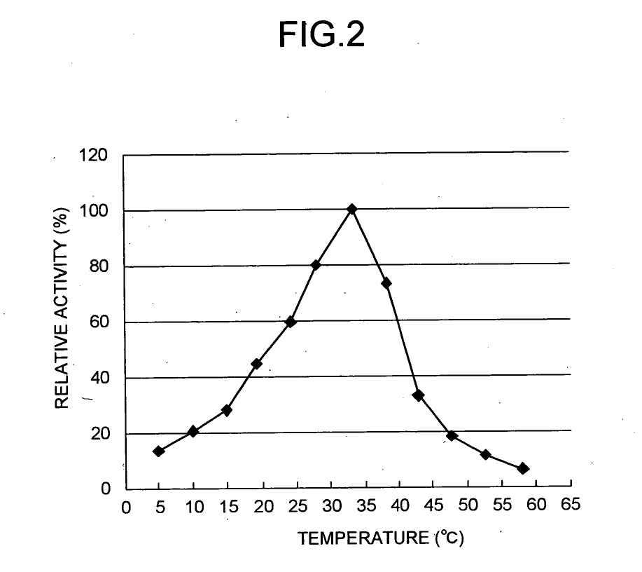

The effects of temperature were examined on the reaction in which L-alanyl-L-glutamine is formed from L-alanine methyl ester hydrochloride and L-glutamine. 1 μl of the same enzyme fraction used in Example 5 was added to 100 μl of 100 mM borate buffer (pH 9.0) containing 100 mM L-alanine methyl ester, 200 mM L-glutamine and 10 mM EDTA and allowed to react for 5 minutes at each temperature to measure the effects of temperature on the reaction. The results based on assigning a value of 100% to the activity at 34° C. are shown in FIG. 2. As a result, the optimum enzyme temperature was 30 to 40° C.

### Example 7

### Enzyme Inhibitors

The effects of inhibitors on the production of L-alanyl-L-glutamine were examined using L-alanine methyl ester hydrochloride and L-glutamine as substrates. 2 μl of the same enzyme fraction used in Example 5 was added to 50 μl of 100 mM borate buffer (pH 9.0) containing each of the enzyme inhibitors shown in Table 2 at 10 mM, and allowed to react at 25° C. for 5 minutes. Note that, o-phenanthroline, phenylmethylsulfonyl fluoride and p-nitrophenyl-p′-guanidinobenzoate were dissolved in methanol to a concentration of 50 mM before use. The enzyme activity under each condition was indicated as the relative activity in the case of assigning a value of 100 to the production of L-alanyl-L-glutamine in the absence of enzyme inhibitor. Those results are shown in Table 2. As a result, among the serine enzyme inhibitors tested, the enzyme was not inhibited by phenylmethylsulfonyl fluoride, but it was inhibited by p-nitrophenyl-p′-guanidinobenzoate.

TABLE 2

| | | Relative activity of |
|---|---|---|
| | | L-Ala-L-Gln production |
| Enzyme inhibitor | | (%) |
| None | | 100 |
| Metal enzyme | EDTA | 96 |
| inhibitor | o-Phenanthroline | 96 |
| SH enzyme | N-Ethyl maleimide | 110 |
| inhibitor | Monoiodoacetate | 101 |
| Serine enzyme | Phenylmethylsulfonyl | 115 |
| inhibitor | fluoride | |
| | 4-(2-Aminoethyl)benzene | 75 |
| | sulfonyl fluoride | |
| | p-Nitrophenyl-p′-guanidino | 0.1 |
| | benzoate | |

### Example 8

### Production of L-Alanyl-L-Glutamine from L-Alanine Methyl Ester and L-Glutamine

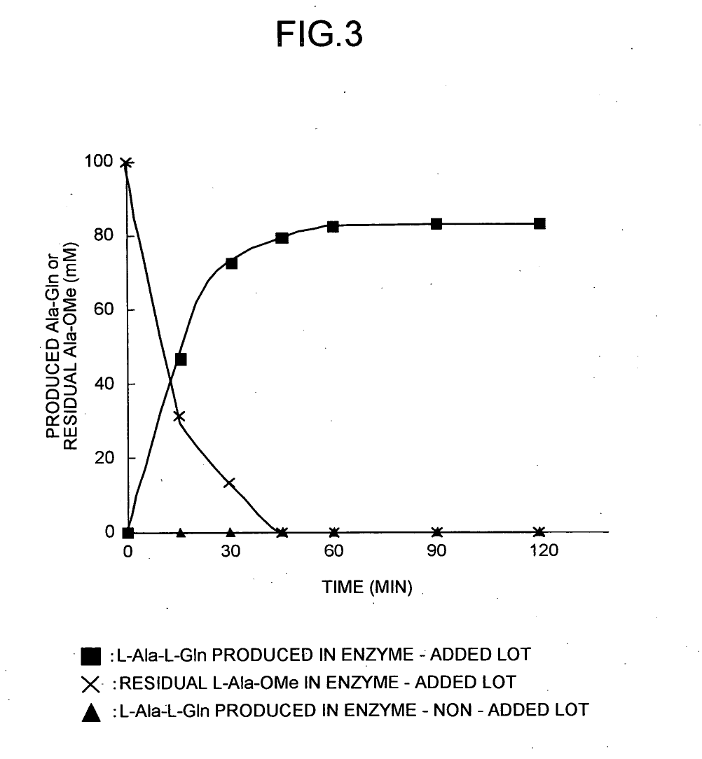

3 μl of the same enzyme fraction as used in Example 5 was added to 100 μl of 100 mM borate buffer (pH 9.0) containing 100 mM L-alanine methyl ester hydrochloride, 200 mM L-glutamine and 10 mM EDTA, and allowed to react at 18° C. As a result, as shown in FIG. 3, 83 mM L-alanyl-L-glutamine (L-Ala-L-Gln) was formed in the case of an enzyme-added lot, and the concentration of by-product L-Ala-L-Ala-L-Gln was 1.3 mM. On the other hand, there was hardly any production of L-Ala-L-Gln observed in an enzyme-non-added lot, and the enzyme concentration was only about 0.07 mM after reacting for 120 minutes.

### Example 9

### Effects of L-Glutamine Concentration on Production of L-Alanyl-L-Glutamine

1 μl of the same enzyme fraction as used in Example 5 was added to 100 μl of 100 mM borate buffer (pH 9.0) containing 100 mM L-alanine methyl ester hydrochloride, L-glutamine at the concentrations shown in Table 3 and 10 mM EDTA, and allowed to react at 18° C. for 2 hours. Those results are shown in Table 3.

TABLE 3

| L-Alanine methyl ester | L-Glutamine | L-Ala-L-Gln |
|---|---|---|
| hydrochloride (mM) | (mM) | (mM) |
| 100 | 100 | 68.2 |
| | 110 | 72.1 |
| | 120 | 73.3 |
| | 130 | 75.1 |
| | 150 | 75.5 |
| | 200 | 82.0 |

### Example 10

### Enzyme Substrate Specificity (1)

Ester specificity was examined in the case of using L-amino acid ester for the carboxy component. 2 μl of the same enzyme fraction as used in Example 5 was added to 100 μl of 100 mM borate buffer (pH 9.0) containing the carboxy components indicated in Table 4 at 100 mM, 200 mM L-glutamine and 10 mM EDTA, and allowed to react at 25° C. for 2 hours. The amounts of L-Ala-L-Gln formed in this reaction are shown in Table 4. HCl represents hydrochloride in Table 4.

TABLE 4

| Carboxy component | L-Ala-L-Gln formed (mM) |
|---|---|
| L-Alanine methyl ester.HCl | 84.3 |
| L-Alanine ethyl ester.HCl | 91.5 |
| L-Alanine isopropyl ester.HCl | 78.9 |
| L-Alanine-t-butyl ester.HCl | 7.5 |

### Example 11

### Enzyme Substrate Specificity (2)

Peptide production was examined in the case of using L-alanine methyl ester for the carboxy component and using various L-amino acids for the amine component. 2 μl of the same enzyme fraction as used in Example 5 was added to 100 μl of 100 mM borate buffer (pH 9.0) containing 100 mM L-alanine methyl ester hydrochloride, the L-amino acids shown in Table 5 at 150 mM and 10 mM EDTA, and allowed to react at 25° C. for 3 hours. The amounts of various peptides formed in this reaction are shown in Table 5. The “+” mark indicates those peptides for which production was confirmed but which were unable to be quantified due to the absence of a standard, while “tr” indicates a trace amount.

TABLE 5

| Amine | Formed | | Amine | Formed | |
|---|---|---|---|---|---|
| component | peptide | (mM) | component | peptide | (mM) |
| Gly | L-Ala-Gly | 13.7 | L-Asn | L-Ala-L-Asn | 65.5 |
| L-Ala | L-Ala-L-Ala | 25.4 | L-Gln | L-Ala-L-Gln | 79.3 |
| L-Val | L-Ala-L-Val | 20.8 | L-Tyr | L-Ala-L-Tyr | 17.6 |
| L-Leu | L-Ala-L-Leu | 45.3 | L-CySH | L-Ala-L-CySH | + |
| L-Ile | L-Ala-L-Ile | 33.9 | L-Lys | L-Ala-L-Lys | 71.8 |
| L-Met | L-Ala-L-Met | 83.3 | L-Arg | L-Ala-L-Arg | 88.0 |
| L-Phe | L-Ala-L-Phe | 74.4 | L-His | L-Ala-L-His | 66.9 |
| L-Trp | L-Ala-L-Trp | 53.9 | L-Asp | L-Ala-L-Asp | 2.1 |
| L-Ser | L-Ala-L-Ser | 62.5 | L-Glu | L-Ala-L-Glu | 42.9 |
| L-Thr | L-Ala-L-Thr | 53.9 | L-Pro | L-Ala-L-Pro | tr |

### Example 12

### Enzyme Substrate Specificity (3)

Peptide production was examined in the case of using various types of L-amino acid methyl esters for the carboxy component and using L-glutamine for the amine component. 2 μl of the same enzyme fraction as used in Example 5 was added to 100 μl of 100 mM borate buffer (pH 9.0) containing the L-amino acid methyl ester hydrochloride salts (AA-OMe.HCl) shown in Table 6 at 100 mM, 150 mM L-glutamine and 10 mM EDTA, and allowed to react at 25° C. for 3 hours. The amounts of various peptides formed in this reaction are shown in Table 6. The “+” mark indicates those peptides for which production was confirmed but which were unable to be quantified due to the absence of a standard, while “tr” indicates a trace amount. Furthermore, Tween-80 was added to the reaction system to a final concentration of 0.1% in the case of using L-Trp-OMe and L-Tyr-OMe.

TABLE 6

| Carboxy | Formed | | Carboxy | Formed | |
|---|---|---|---|---|---|
| component | peptide | (mM) | component | peptide | (mM) |
| Gly-OMe | Gly-L-Gln | 54.7 | L-Tyr-OMe | L-Tyr-L-Gln | 3.4 |
| L-Ala-OMe | L-Ala-L-Gln | 74.6 | CySH-OMe | L-CySH-L-Gln | + |
| L-Val-OMe | L-Val-L-Gln | 15.4 | L-Lys-OMe | L-Lys-L-Gln | + |
| L-Leu-OMe | L-Leu-L-Gln | + | L-Arg-OMe | L-Arg-L-Gln | 7.1 |
| L-Ile-OMe | L-Ile-L-Gln | 8.4 | L-His-OMe | L-His-L-Gln | + |
| L-Met-OMe | L-Met-L-Gln | 12.0 | L-Asp-α- | α-L-Asp-L-Gln | tr |
| | | | OMe | | |
| L-Phe-OMe | L-Phe-L-Gln | 0.9 | L-Asp-β- | β-L-Asp-L-Gln | tr |
| | | | OMe | | |
| L-Trp-OMe | L-Trp-L-Gln | + | L-Glu-α- | α-L-Glu-L-Gln | + |
| | | | OMe | | |
| L-Ser-OMe | L-Ser-L-Gln | 24.0 | L-Glu-γ- | γ-L-Glu-L-Gln | + |
| | | | OMe | | |
| L-Thr-OMe | L-Thr-L-Gln | 81.9 | L-Pro-OMe | L-Pro-L-Gln | 2.2 |
| L-Asn-OMe | L-Asn-L-Gln | + | | | |
| L-Gln-OMe | L-Gln-L-Gln | 0.3 | | | |

Hydrochloride salts were used for all the carboxy components.

### Example 13

### Enzyme Substrate Specificity (4)

Peptide production was examined in the case of using various L-amino acid methyl esters for the carboxy component and various L-amino acids for the amine component. 2 μl of the same enzyme fraction as used in Example 5 was added to 100 μl of 100 mM borate buffer (pH 9.0) containing the L-amino acid methyl ester hydrochloride salts (AA-OMe.HCl) shown in Table 7 at 100 mM, the L-amino acids shown in Table 7 at 150 mM and 10 mM EDTA, and allowed to react at 25° C. for 3 hours. The amounts formed of various peptides formed in this reaction are shown in Table 7. The “tr” indicates a trace amount. Furthermore, Tween-80 was added to the reaction system to a final concentration of 0.1% in the case of using L-Trp-OMe. The “+” mark indicates those peptides for which production was confirmed but which were unable to be quantified due to the absence of a standard.

TABLE 7

| Carboxy | Amine | Formed | | Carboxy | Amine | Formed | |
|---|---|---|---|---|---|---|---|
| component | component | peptide | (mM) | component | component | peptide | (mM) |
| Gly-OMe | L-CySH | Gly-L- | 45.6 | L-Met-OMe | L-Ser | L-Met-L- | 12.8 |
| | | CySH | | | | | |
| | L-Arg | Gly-L- | 25.5 | | L-Met | L-Met-L- | 25.0 |
| | | Arg | | | | | |
| | L-Phe | Gly-L- | 44.0 | | L-Phe | L-Met-L- | 34.0 |
| | | Phe | | | | | |
| | L-His | Gly-L- | 31.6 | L-Ile-OMe | L-Ser | L-Ile-L- | 17.2 |
| | | His | | | | Ser | |
| | L-Lys | Gly-L- | 9.8 | | L-Met | L-Ile-L- | 10.0 |
| | | Lys | | | | Met | |
| | L-Ser | Gly-L- | 44.2 | | L-Phe | L-Ile-L- | 5.2 |
| | | Ser | | | | Phe | |
| L-Thr-OMe | Gly | L-Thr- | 9.4 | L-Arg-OMe | L-Ser | L-Arg-L- | 3.6 |
| | | Gly | | | | Ser | |
| | L-Ala | L-Thr-L- | 9.4 | | L-Met | L-Arg-L- | 0.7 |
| | | | | | | Met | |
| | L-Val | L-Thr-L- | 0.7 | | L-Phe | L-Arg-L- | 1.9 |
| | | Val | | | | Phe | |
| | L-Leu | L-Thr-L- | 28.4 | L-Leu-OMe | L-Met | L-Leu-L- | 12.2 |
| | | Leu | | | | | |
| | L-Met | L-Thr-L- | 38.6 | L-Trp-OMe | L-Met | L-Trp-L- | 4.1 |
| | | Met | | | | Met | |
| | L-Ser | L-Thr-L- | 58.2 | L-Lys-OMe | L-Met | L-Lys-L- | 6.8 |
| | | Ser | | | | Met | |
| L-Ser-OMe | L-Ser | L-Ser-L- | 38.0 | L-His-OMe | L-Met | L-His-L- | 6.5 |
| | | Ser | | | | Met | |
| | L-Met | L-Ser-L- | 12.5 | L-Asn-OMe | L-Glu | L-Asn-L- | 10.2 |
| | | Met | | | | | |
| | L-Phe | L-Ser-L- | 20.3 | | | | |
| | | Phe | | | | | |
| L-Val-OMe | L-Ser | L-Val-L- | 30.8 | | | | |
| | | Ser | | | | | |
| | L-Met | L-Val-L- | 10.3 | | | | |
| | | Met | | | | | |
| | L-Phe | L-Val-L- | 6.1 | | | | |
| | | Phe | | | | | |

Hydrochloride salts were used for all the carboxy components.

### Example 14

### Enzyme Substrate Specificity (5)

Peptide production was examined in the case of using the L or D forms of various amino acid methyl esters for the carboxy component, and the L or D forms of various amino acids for the amine component. 2 μl of the same enzyme fraction as used in Example 5 was added to 100 μl of 100 mM borate buffer (pH 9.0) containing the various amino acid methyl ester hydrochloride salts (AA-OMe HCl) shown in Table 8 at 100 mM, the various amino acids shown in Table 8 at 150 mM and 10 mM EDTA, and allowed to react at 25° C. for 3 hours. The amounts of various peptides formed in this reaction are shown in Table 8. The “tr” indicates a trace amount.

TABLE 8

| Carboxy | Amine | | |
|---|---|---|---|
| component | component | Formed peptide | (mM) |
| D-Ala-OMe | L-Gln | D-Ala-L-Gln | 69.3 |
| D-Ala-OMe | L-Ser | D-Ala-L-Ser | 20.3 |
| D-Thr-OMe | | D-Thr-L-Ser | 1.0 |
| D-Ser-OMe | | D-Ser-L-Ser | 3.3 |
| D-Val-OMe | | D-Val-L-Ser | 0.6 |
| D-Met-OMe | | D-Met-L-Ser | 5.1 |
| L-Ala-OMe | D-Gln | L-Ala-D-Gln | 0.3 |
| L-Ala-OMe | D-Ser | L-Ala-D-Ser | 5.4 |
| L-Thr-OMe | | L-Thr-D-Ser | 6.9 |
| L-Ser-OMe | | L-Ser-D-Ser | 16.2 |
| L-Val-OMe | | L-Val-D-Ser | 1.4 |
| L-Met-OMe | | L-Met-D-Ser | 1.9 |
| D-Ala-OMe | D-Gln | D-Ala-D-Gln | tr |
| D-Ala-OMe | D-Ser | D-Ala-D-Ser | 0.2 |
| D-Thr-OMe | | D-Thr-D-Ser | 1.1 |
| D-Ser-OMe | | D-Ser-D-Ser | 2.5 |
| D-Val-OMe | | D-Val-D-Ser | 0.5 |
| D-Met-OMe | | D-Met-D-Ser | 2.7 |

Hydrochloride salts were used for all the carboxy components.

### Example 15

### Enzyme Substrate Specificity (6)

Peptide production was examined using various L-amino acid amides for the carboxy component, and various L-amino acids for the amine component. 2 μl of the same enzyme fraction as that used in Example 5 was added to 100 μl of 100 mM borate buffer (pH 9.0) containing the L-amino acid amide hydrochloride salt (M-NH₂.HCl) shown in Table 9 at 100 mM, the L-amino acids shown in Table 9 at 150 mM and 10 mM EDTA, and allowed to react at 25° C. for 3 hours. The amounts of various peptides formed in this reaction are shown in Table 9.

TABLE 9

| Carboxy | Amine | Formed | |
|---|---|---|---|
| component | component | peptide | (mM) |
| L-Phe-NH₂ | L-Gln | L-Phe-L-Gln | 0.2 |
| L-Phe-NH₂ | L-Ser | L-Phe-L-Ser | 0.6 |
| L-Ala-NH₂ | L-Gln | L-Ala-L-Gln | 7.6 |
| L-Ala-NH₂ | L-Met | L-Ala-L-Met | 3.4 |
| L-Ala-NH₂ | L-His | L-Ala-L-His | 3.9 |
| L-Thr-NH₂ | L-Gln | L-Thr-L-Gln | 0.3 |

### Example 16

### Enzyme Substrate Specificity (7)

Peptide production was examined in the case of using various L-alanine methyl esters for the carboxy component and C-protected L-amino acids for the amine component. 2 μl of the same enzyme fraction as used in Example 5 was added to 100 μl of 100 mM borate buffer (pH 9.0) containing the L-alanine methyl ester hydrochloride hydrochloride salt (Ala-OMe.HCl) shown in Table 10 at 100 mM, the L-amino acid amide hydrochloride salts shown in Table 10 at 150 mM and 10 mM EDTA, and allowed to react at 25° C. for 3 hours. The amounts of various peptides formed in this reaction are shown in Table 10.

TABLE 10

| Carboxy | | | |
|---|---|---|---|
| component | Amine component | Formed peptide | (mM) |
| L-Ala-OMe | Gly-NH₂ | L-Ala-Gly-NH₂ | 7.4 |
| | L-Ala-NH₂ | L-Ala-L-Ala-NH₂ | 8.3 |
| | L-Phe-NH₂ | L-Ala-L-Phe-NH₂ | 12.2 |

### Example 17

### Enzyme Substrate Specificity (8)

Peptide production was examined in the case of using various amino acid methyl esters for the carboxy component and methylamine for the amine component. 2 μl of the same enzyme fraction as used in Example 5 was added to 100 μl of 100 mM borate buffer (pH 9.0) containing the amino acid methyl ester hydrochloride salt (M-OMe.HCl) shown in Table 11 at 100 mM, the methylamine shown in Table 11 at 150 mM and 10 mM EDTA, and allowed to react at 25° C. for 3 hours. The amounts of various peptides formed in this reaction are shown in Table 11.

TABLE 11

| Carboxy component | Amine component | Formed peptide | (mM) |
|---|---|---|---|
| Gly-OMe | Methylamine | Gly-methylamine | 1.1 |
| L-Thr-OMe | | L-Thr-methylamine | 0.2 |
| L-Ala-OMe | | L-Ala-methylamine | 0.3 |

### Example 18

### Enzyme Substrate Specificity (9)

Peptide production was examined in the case of using 62 -amino acid ester for the carboxy component or β-amino acid for the amine component. 2 μl of the same enzyme fraction as used in Example 5 was added to 100 μl of 100 mM borate buffer (pH 9.0) containing the carboxy components shown in Table 12 at 100 mM, the amine components shown in Table 12 at 150 mM and 10 mM EDTA, and allowed to react at 25° C. for 3 hours. The amounts of 5 various peptides formed in this reaction are shown in Table 12. The “tr” indicates a trace amount.

TABLE 12

| Carboxy component | Amine component | Formed peptide | (mM) |
|---|---|---|---|
| Gly-OMe | β-Ala | Gly-β-Ala | 2.2 |
| Gly-OMe | β-Phe | Gly-β-Phe | 0.4 |
| L-Ala-OMe | β-Ala | Ala-β-Ala | 7.7 |
| L-Ala-OMe | β-Phe | Ala-β-Phe | 1.4 |
| L-Thr-OMe | β-Ala | Thr-β-Ala | 3.2 |
| L-Thr-OMe | β-Phe | Thr-β-Phe | 1.4 |
| β-Ala-OMe | L-α-Ala | β-Ala-L-α-Ala | tr |
| β-Ala-OMe | β-Ala | β-Ala-β-Ala | 0.2 |
| β-Ala-OMe | L-Gln | β-Ala-L-Gln | 0.6 |
| β-Ala-OMe | L-Ser | β-Ala-L-Ser | 3.2 |

Hydrochloride salts were used for all the carboxy components.

### Example 19

### Enzyme Substrate Specificity (10)

Oligopeptide production was examined in the case of using L-amino acid ester for the carboxy component and peptide for the amine component. 2 μl of the same enzyme fraction as used in Example 5 was added to 100 μl of 100 mM borate buffer (pH 9.0) containing the carboxy components shown in Table 13 at 100 mM, the amine components shown in Table 13 at 150 mM and 10 mM EDTA, and allowed to react at 25° C. for 3 hours. The amounts of various peptides formed in this reaction are shown in Table 13. As a result, it was clearly demonstrated that the present enzyme can form not only dipeptide, but also long-chain peptides by using a peptide for the amine component.

As has been indicated in the aforementioned Examples 9 to 20, the present enzyme obtained from Empedobacter brevis strain FERM BP-1 8545 was determined to have extremely broad substrate specificity.

TABLE 13

| Carboxy | | | |
|---|---|---|---|
| component | Amine component | Produced peptide | (mM) |
| L-Ala-OMe | L-Ala | L-Ala-L-Ala | 28.7 |
| | L-Ala-L-Ala | L-Ala-L-Ala-L-Ala | 57.5 |
| | L-Ala-L-Ala-L-Ala | L-Ala-L-Ala-L-Ala-L-Ala | 44.5 |
| | L-Ala-L-Ala-L-Ala-L-Ala | L-Ala-L-Ala-L-Ala-L-Ala-L-Ala | 34.8 |
| | L-Ala-L-Ala-L-Ala-L-Ala-L-Ala | L-Ala-L-Ala-L-Ala-L-Ala-L-Ala-L-Ala | 1.4* |
| | L-Ala-L-Gln | L-Ala-L-Ala-L-Gln | 15.2 |
| | Gly-L-Ala | L-Ala-Gly-L-Ala | 25.9 |
| | Gly-Gly | L-Ala-Gly-Gly | 41.7 |
| | L-His-L-Ala | L-Ala-L-His-L-Ala | 55.9 |
| | L-Leu-L-Ala | L-Ala-L-Leu-L-Ala | 48.3 |
| | L-Phe-L-Ala | L-Ala-L-Phe-L-Ala | 49.7 |
| | L-Phe-Gly | L-Ala-L-Phe-Gly | 43.7 |
| Gly-OMe | L-Ala-L-Tyr | Gly-L-Ala-L-Tyr | 1.7 |
| | Gly-L-Gln | Gly-Gly-L-Gln | 7.2 |
| | Gly-L-Tyr-L-Ala | Gly-Gly-L-Tyr-L-Ala | 44.2 |
| L-Thr-OMe | Gly-Gly | L-Thr-Gly-Gly | 83.0 |

*Since the solubility of L-Ala-L-Ala-L-Ala-L-Ala-L-Ala was low, the carboxy component was used at a concentration of 10 mM and the amine component was used at 15 mM in this reaction system. The other conditions were the same as those explained in the example. Hydrochloride salts were used for all the carboxy components.

### Example 20

### Comparison of Ability to Catalyze Peptide Formation with Known Enzymes

The peptide-forming ability of the present enzyme was compared with that of known enzymes. Carboxypeptidase Y described in EP 278787A1 and the thiol endopeptidases (ficin, papain, bromelain, and chymopapain) described in EP 359399B1 were used as the known enzymes, and they were used in the form of purified enzymes (manufactured by Sigma). The enzyme homogeneously purified in Example 3 was used as a source of the present enzyme of the present invention. These enzymes were added to a reaction system in the protein amounts shown in Table 14. The reaction was carried out by adding the enzyme to 100 μl of borate buffer (pH 9.0) containing 100 mM L-alanine methyl ester hydrochloride salt and 200 mM L-glutamine and allowing the resultant to react at 25° C. Note that the carboxypeptidase used was one dissolved in 10 mM acetate buffer (pH 5.0) containing 1 mM EDTA, while the thiol endopeptidase used was one dissolved in 10 mM acetate buffer (pH 5.0) containing 2 mM EDTA, 0.1 M KCl, and 5 mM dithiothreitol. The ratios of the production rates of L-alanyl-L-glutamine by these enzymes are shown in Table 14.

As a result, the production of an extremely trace small amount of L-alanyl-L-glutamine was observed even in the absence of enzymes, while a slight increase in the production rate was observed in the section where carboxypeptidase- or thiol endopeptidase-added lot as compared with the enzyme-non-added lot. In contrast, an overwhelmingly higher rate of production of L-alanyl-L-glutamine was observed in the enzyme-added lot, and that rate of production was about 5,000 to 100,000 times higher than those of carboxypeptidase Y and of thiol endopeptidase. As has been described above, the present enzyme was verified to have an extremely high peptide production rate unlike any known enzyme in the prior art. Furthermore, the enzyme of the present invention is a dimer having a molecular weight of about 75,000. In contrast, the molecular weight of the carboxypeptidase Y has been reported to be about 61,000, while the molecular weight of thiol endopeptidase has been reported to be about 23,000 to 36,000. Thus, the L-alanyl-L-glutamine production rate of the enzyme of the present invention as compared to those of the carboxypeptidase Y and the thiol endopeptidase is even greater when the rate is expressed per molecular weight than when it is expressed per unit weight as indicated in the examples.

TABLE 14

| | | | Ratio of |
|---|---|---|---|
| | Amount of | L-Ala-L-Gln | L-Ala-L-Gln |
| | enzyme | production | production rate |
| | added | rate | per enzyme unit |
| Enzyme | (protein mg/ml) | (mM/min) | weight |
| No enzyme | 0 | 0.0006 | |
| Carboxypeptidase Y | 0.61 | 0.0257 | 0.0191 |
| Ficin | 2.60 | 0.0096 | 0.0017 |
| Papain | 2.30 | 0.0106 | 0.0021 |
| Bromelain | 2.80 | 0.0062 | 0.0010 |
| Chymopapain | 3.60 | 0.0100 | 0.0013 |
| Enzyme of present | 0.02 | 4.4000 | 100.0 |
| invention | | | |

### Example 21

### Production of L-Alanyl-L-Glutamine Using Microbial Cell of Sphingobacterium sp.

A 50 ml medium (pH 7.0) containing 5 g of glucose, 5 g of ammonium sulfate, 1 g of monopotassium phosphate, 3 g of dipotassium phosphate, 0.5 g of magnesium sulfate, 10 g of yeast extract, and 10 g of peptone in 1 L was transferred to a 500 mL Sakaguchi flask and sterilized at 11 5° C. for 15 minutes for culturing Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002). This medium was then inoculated with one loopful cells of Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002) cultured at 30° C. for 24 hours in a slant agar medium (agar: 20 g/L, pH 7.0) containing 5 g of glucose, 10 g of yeast extract, 10 g of peptone and 5 g of NaCl in 1 L, followed by shake culturing at 30° C. for 20 hours and 120 strokes/minute. 1 ml of this culture broth was then added to the aforementioned medium (50 ml/500 mL Sakaguchi flask) and cultured at 30° C. for 18 hours. Following completion of the culturing, the microbial cells were separated from the culture broth by centrifugation and suspended in 0.1 M borate buffer (pH 9.0) containing 10 mM EDTA to 100 g/L as wet microbial cells. 0.1 mL of 100 mM borate buffer (pH 9.0) containing 10 mM EDTA, 200 mM L-alanyl methyl ester hydrochloride and 400 mM L-glutamine was then added to 0.1 mL of this microbial cell suspension, and after bringing to a final volume of 0.2 mL, was allowed to react at 25° C. for 120 minutes. The amount of L-alanyl-L-glutamine formed at this time was 62 mM.

### Example 22

### Purification of Enzyme from Sphingobacterium sp.

The following procedure after centrifugation was carried out either on ice or at 4° C. Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002) was cultured in the same manner as Example 21, and the microbial cells were collected by centrifugation (10,000 rpm, 15 minutes). After washing 2 g of microbial cells with 20 mM Tris-HCl buffer (pH 7.6), they were suspended in 8 ml of the same buffer and subjected to ultrasonic crushing treatment for 45 minutes at 195 W. This ultrasonic crushed suspension was then centrifuged (10,000 rpm, 30 minutes) to remove the crushed cell fragments and obtain a supernatant. This supernatant was dialyzed overnight against 20 mM Tris-HCl buffer (pH 7.6) followed by removal of the insoluble fraction by ultracentrifugation (50,000 rpm, 30 minutes) to obtain a soluble fraction in the form of the supernatant liquid. The resulting soluble fraction was applied to a Q-Sepharose HP column (manufactured by Amersham) pre-equilibrated with Tris-HCl buffer (pH 7.6), and the active fraction was collected from the non-adsorbed fraction. This active fraction was dialyzed overnight against 20 mM acetate buffer (pH 5.0) followed by removal of the insoluble fraction by centrifugation (10,000 rpm, 30 minutes) to obtain a dialyzed fraction in the form of the supernatant liquid. This dialyzed fraction was then applied to an SP-Sepharose HP column (manufactured by Amersham) pre-equilibrated with 20 mM acetate buffer (pH 5.0) to obtain the active fraction in which enzyme was eluted at a linear concentration gradient of the same buffer containing 0 to 1 M NaCl.

### Example 23

### Production of L-Alanyl-L-Glutamine Using Enzyme Fraction

10 μl of the SP-Sepharose HP fraction (about 27 U/ml) purified in Example 22 was added to 90 μl of 111 mM borate buffer (pH 9.0) containing 111 mM L-alanine methyl ester hydrochloride, 222 mM L-glutamine and 11 mM EDTA, and allowed to react at 25° C. for 120 minutes. As a result, 73 mM of L-alanyl-L-glutamine was formed in the enzyme-added lot. On the other hand, there was hardly any production of L-Ala-L-Glu observed in the enzyme-non-added lot, and the production amount was only about 0.07 mM after reacting for 120 minutes.

### Example 24

### Enzyme Substrate Specificity (11)

Substrate specificity was examined for enzyme derived from Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002). 100 μl of 100 mM borate buffer (pH 9.0) containing the various carboxy components at a final concentration of 100 mM and the various amine components at a final concentration of 150 mM shown in Tables 15-1 to 15-4, the SP-Sepharose HP fraction enzyme purified in Example 22 (addition of 0.33 units in the reaction liquid) and 10 mM EDTA were allowed to react at 25° C. for 1.5 hours. The amounts of various peptides formed in this reaction are shown in Table 15. The “+” mark indicates those peptides for which production was confirmed but which were unable to be quantified due to the absence of a standard, while “tr” indicates a trace amount. Furthermore, Tween-80 was added to the reaction system to a final concentration of 0.1% in the case of using L-Tyr-OMe. In addition, hydrochloride salts were used for all carboxy components.

TABLE 15-1

| Carboxy | Amine | Produced | |
|---|---|---|---|
| component | component | peptide | (mM) |
| L-Ala-OMe | Gly | L-Ala-Gly | 11.1 |
| | L-Ala | L-Ala-L-Ala | 13.1 |
| | L-Val | L-Ala-L-Val | 10.9 |
| | L-Leu | L-Ala-L-Leu | 33.0 |
| | L-Ile | L-Ala-L-Ile | 24.7 |
| | L-Met | L-Ala-L-Met | 86.9 |
| | L-Pro | L-Ala-L-Pro | 1.5 |
| | L-Phe | L-Ala-L-Phe | 69.5 |
| | L-Trp | L-Ala-L-Trp | 46.0 |
| | L-Thr | L-Ala-L-Thr | 47.3 |
| | L-Asn | L-Ala-L-Asn | 52.3 |
| | L-Tyr | L-Ala-L-Tyr | 11.1 |
| | L-CySH | L-Ala-L-CySH | + |
| | L-Lys | L-Ala-L-Lys | 71.2 |
| | L-Arg | L-Ala-L-Arg | 72.2 |
| | L-His | L-Ala-L-His | 73.6 |
| | L-Asp | L-Ala-L-Asp | 2.3 |
| | L-Glu | L-Ala-L-Glu | 39.1 |
| | L-Ser | L-Ala-L-Ser | 43.8 |
| | D-Ser | L-Ala-D-Ser | 3.3 |
| D-Ala-OMe | L-Ser | D-Ala-L-Ser | 24.1 |
| | D-Ser | D-Ala-D-Ser | 5.5 |

TABLE 15-2

| Carboxy | Amine | Produced | |
|---|---|---|---|
| component | component | peptide | (mM) |
| L-Thr-OMe | L-Gln | L-Thr-L-Gln | 36.1 |
| Gly-OMe | | Gly-L-Gln | 61.1 |
| L-Ser-OMe | | L-Ser-L-Gln | 12.9 |
| L-Val-OMe | | L-Val-L-Gln | 8.2 |
| L-Met-OMe | | L-Met-L-Gln | 32.6 |
| L-Ile-OMe | | L-Ile-L-Gln | 6.4 |
| L-Arg-OMe | | L-Arg-L-Gln | 17.2 |
| L-Tyr-OMe | | L-Tyr-L-Gln | 0.6 |
| L-Pro-OMe | | L-Pro-L-Gln | 1.8 |
| L-Phe-OMe | | L-Phe-L-Gln | 0.8 |
| L-Gln-OMe | | L-Gln-L-Gln | 0.1 |
| Asp-α-OMe | | α-L-Asp-L-Gln | 0.05 |

TABLE 15-3

| Carboxy | Amine | Produced | |
|---|---|---|---|
| component | component | peptide | (mM) |
| L-Thr-OMe | Gly | L-Thr-Gly | 0.4 |
| | L-Ala | L-Thr-L-Ala | 5.8 |
| | L-Val | L-Thr-L-Val | 1.3 |
| | L-Leu | L-Thr-L-Leu | 15.3 |
| | L-Met | L-Thr-L-Met | 28.9 |
| Gly-OMe | L-Arg | Gly-L-Arg | 17.9 |
| | L-Phe | Gly-L-Phe | 20.0 |
| | L-His | Gly-L-His | 36.2 |
| | L-Lys | Gly-L-Lys | 48.2 |
| | L-Ser | Gly-L-Ser | 53.8 |
| L-Ser-OMe | L-Ser | L-Ser-L-Ser | 9.9 |
| | L-Met | L-Ser-L-Met | 7.6 |
| | L-Phe | L-Ser-L-Phe | 4.3 |
| L-Val-OMe | L-Ser | L-Val-L-Ser | 31.9 |
| | L-Met | L-Val-L-Met | 6.8 |
| | L-Phe | L-Val-L-Phe | 1.0 |
| L-Met-OMe | L-Ser | L-Met-L-Ser | 25.3 |
| | L-Met | L-Met-L-Met | 28.4 |
| | L-Phe | L-Met-L-Phe | 8.9 |
| L-Ile-OMe | L-Ser | L-Ile-L-Ser | 17.3 |
| | L-Met | L-Ile-L-Met | 5.1 |
| | L-Phe | L-Ile-L-Phe | 1.5 |
| L-Arg-OMe | L-Ser | L-Arg-L-Ser | 2.2 |
| | L-Met | L-Arg-L-Met | tr |
| | L-Phe | L-Arg-L-Phe | tr |

TABLE 15-4

| Carboxy | Amine | | |
|---|---|---|---|
| component | component | Produced peptide | (mM) |
| L-Ala-OMe | Gly amide | L-Ala-Gly amide | 15.1 |
| | L-Ala amide | L-Ala-L-Ala amide | 9.2 |
| | L-Phe amide | L-Ala-Phe amide | 27.1 |
| L-Ala-OMe | Methylamine | L-Ala-methylamine | 0.6 |
| L-Thr-OMe | | L-Thr-methylamine | 0.3 |
| Gly-OMe | | Gly-methylamine | 1.0 |
| L-Ala amide | L-Gln | L-Ala-L-Gln | 0.3 |
| | L-Met | L-Ala-L-Met | tr |
| | L-His | L-Ala-L-His | tr |

Hydrochloride salts were used for all the carboxy components.

### Example 25

### Enzyme Substrate Specificity (12)

Substrate specificity with respect to oligopeptide production was examined for enzyme derived from Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002). 100 μl of 100 mM borate buffer (pH 9.0) containing the various carboxy components at a final concentration of 100 mM and the various amine components at a final concentration of 150 mM shown in Table 16, the SP-Sepharose HP fraction enzyme purified in Example 22 (addition of 0.33 units in the reaction liquid) and 10 mM EDTA were allowed to react for 1.5 hours at 25° C. The amounts of each oligopeptide formed in this reaction are shown in Table 16. Furthermore, hydrochloride salts were used for all the carboxy components.

TABLE 16

| Carboxy | | | |
|---|---|---|---|
| component | Amine component | Produced peptide | (mM) |
| L-Ala-OMe | L-Ala | L-Ala-L-Ala | 25.6 |
| | L-Ala-L-Ala | L-Ala-L-Ala-L-Ala | 41.1 |
| | L-Ala-L-Ala-L-Ala | L-Ala-L-Ala-L-Ala-L-Ala | 30.1 |
| | L-Ala-L-Ala-L- | L-Ala-L-Ala-L-Ala-L- | 22.8 |
| | Ala-L-Ala | Ala-L-Ala | |
| | Gly-Gly | L-Ala-Gly-Gly | 33.7 |
| | Gly-Ala | L-Ala-Gly-L-Ala | 35.1 |
| | L-His-L-Ala | L-Ala-L-His-L-Ala | 58.0 |
| | L-Phe-Gly | L-Ala-L-Phe-Gly | 34.0 |
| | L-Leu-L-Ala | L-Ala-L-Leu-L-Ala | 40.7 |
| | L-Phe-L-Ala | L-Ala-L-Phe-L-Ala | 24.8 |
| L-Thr-OMe | Gly-Gly | L-Thr-Gly-Gly | 8.4 |
| Gly-OMe | L-Ala-L-Tyr | Gly-L-Ala-L-Tyr | 0.6 |

### Example 26

### Enzyme Substrate Specificity (13)

Substrate specificity was additionally assessed using the same enzyme fraction as that used in Example 5.

TABLE 17

| Carboxy component | (mM) | Amine component | (mM) | Produced peptide | (mM) | Reaction time (hr) |
|---|---|---|---|---|---|---|
| HH-Ala-OMe | 50 mM | H-p-F-Phe-OH | 50 mM | H-Ala-p-F-Phe-OH | 21.9 mM | 3 |
| H-Ala-OMe | 40 mM | H-Cl-F-Phe-OH | 40 mM | H-Ala-Cl-F-Phe-OH | 20.8 mM | 3 |
| H-Ala-OMe | 40 mM | H-p-NO₂-Phe-OH | 40 mM | H-Ala-p-NO₂-Phe-OH | 27.5 mM | 3 |
| H-Ala-OMe | 100 mM | H-t-Leu-OH | 150 mM | H-Ala-t-Leu-OH | 0.4 mM | 3 |
| H-Ala-OMe | 20 mM | H-2-Nal-OH | 20 mM | H-Ala-2-Nal-OH | + | 3 |
| H-p-F-Phe-OMe | 100 mM | H-Gln-OH | 150 mM | H-p-F-Phe-H-Gln-OH | tr | 3 |
| H-Cl-F-Phe-OMe | 25 mM | H-Gln-OH | 50 mM | H-Cl-F-Phe-H-Gln-OH | tr | 3 |
| H-p-NO₂-Phe-OMe | 40 mM | H-Gln-OH | 40 mM | H-p-NO₂-Phe-H-Gln-OH | 1.1 mM | 3 |
| H-t-Leu-OMe | 100 mM | H-Gln-OH | 150 mM | H-t-Leu-H-Gln-OH | tr | 3 |
| H-2-Nal-OMe | 40 mM | H-Gln-OH | 40 mM | H-2-Nal-H-Gln-OH | tr | 3 |
| H-Aib-OMe | 100 mM | H-Gln-OH | 150 mM | H-Aib-H-Gln-OH | 18.8 mM | 3 |
| H-N-Me-Ala-OMe | 100 mM | H-Gln-OH | 150 mM | H-N-Me-Ala-H-Gln-OH | 0.5 mM | 3 |
| H-Aib-OMe | 100 mM | H-Phe-OH | 150 mM | H-Aib-Phe-OH | 17.2 mM | 3 |
| H-CHA-OMe | 40 mM | H-Phe-OH | 40 mM | H-CHA-Phe-OH | + | 3 |
| H-N-Me-Ala-OMe | 100 mM | H-Phe-OH | 150 mM | H-N-Me-Ala-Phe-OH | tr | 3 |
| H-Ala-OMe | 100 mM | H-Ser(tBu)-OH | 150 mM | H-Ala-Ser(tBu)-OH | 48.8 mM | 2 |
| H-Ser(tBu)-OMe | 100 mM | H-Gln-OH | 150 mM | H-Ser(tBu)-Gln-OH | tr | 2 |
| H-Ala-OMe | 100 mM | H-Asp(OtBu)-OH | 150 mM | H-Ala-Asp(OtBu)-OH | 62.6 mM | 2 |
| H-Asp(OtBu)-OMe | 100 mM | H-Gln-OH | 150 mM | H-Asp(OtBu)-Gln-OH | 0.9 mM | 2 |
| H-Ala-OMe | 100 mM | H-Lys(Boc)-OH | 150 mM | H-Ala-Lys(Boc)-OH | 51.0 mM | 2 |
| H-Lys(Boc)-OMe | 100 mM | H-Gln-OH | 150 mM | H-Lys(Boc)-Gln-OH | + | 2 |

100 μl of reaction solutions consisting of 100 mM borate buffer (pH 9.0) containing various carboxy components and amine components at the final concentrations shown in Table 17, enzyme (addition of 0.1 unit in reaction solution) and 10 mM EDTA were allowed to react at 25° C. for the reaction times shown in Table 17. The amounts of various peptides formed in the reactions are shown in Table 17. The “+” mark indicates those for which production was confirmed but which were unable to be quantified due to the absence of a standard, while “tr” indicates a trace amount.

Abbreviations

H-Ala-OMe: L-alanine methyl ester hydrochloride

H-p-F-Phe-OMe: p-fluoro-L-phenylalanine methyl ester hydrochloride

H—Cl—F-Phe-OMe: p-chloro-L-phenylalanine methyl ester hydrochloride

H-p-NO₂-Phe-OMe: p-nitro-L-phenylalanine methyl ester hydrochloride

H-t-Leu-OMe: tert-L-leucine methyl ester hydrochloride

H-2-Nal-OMe: 3-(2-naphthyl)-L-alanine methyl ester hydrochloride

H-Aib-OMe: α-aminoisobutyric acid methyl ester hydrochloride

H—N-Me-Ala-OMe: N-methyl-L-alanine methyl ester hydrochloride

H-CHA-OMe: β-cyclohexyl-L-alanine methyl ester hydrochloride

H-Ser(tBu)-OMe: O-tert-butyl-L-serine methyl ester hydrochloride

H-Asp(OtBu)-OMe: L-aspartic acid β-tert-butyl ester α-methyl ester hydrochloride

H-Lys(Boc)-OMe: N-ε-tert-butoxycarbonyl-L-lysine methyl ester hydrochloride

H-p-F-Phe-OH: p-fluoro-L-phenylalanine

H—Cl—F-Phe-OH: p-chloro-L-phenylalanine

H-p-NO₂-Phe-OH: p-nitro-L-phenylalanine

H-t-Leu-OH: tert-L-leucine

H-2-Nal-OH: 3-(2-naphthyl)-L-alanine

H-Gln-OH: L-glutamine

H-Phe-OH: L-phenylalanine

H-Ser(tBu)-OH: O-tert-butyl-L-serine

H-Asp(OtBu)-OH: L-aspartic acid β-tert-butyl ester

H-Lys(Boc)-OH: N-ε-tert-butoxycarbonyl-L-lysine

### Example 27

### Enzyme Substrate Specificity (14)

Substrate specificity with respect to oligopeptide production was assessed using the same enzyme fraction as Example 5 (derived from Empedobacter brevis). 100 μl of reaction solutions consisting of 100 mM borate buffer (pH 9.0) containing various carboxy components and amine components at the final concentrations shown in Table 18, enzyme (the numbers of units added to the reaction solution are described in Table 18) and 10 mM EDTA were allowed to react at 25° C. for 3 hours. The amounts of various oligopeptides formed in the reactions are shown in Table 18. A “+” mark indicates those for which production was confirmed but which were unable to be quantified due to the absence of a standard, while “tr” indicates a trace amount. It should be noted that hydrochloride salts were used for all the carboxy components.

TABLE 18

| | | Amount | | |
|---|---|---|---|---|
| | | of | | |
| Carboxy | | enzyme | | |
| component | Amine component | (unit) | Produced peptide | (mM) |
| Gly-OMe | L-Phe-L-Met | 1.0 | Gly-Phe-Met | 13.3 |
| L-Ala-OMe | L-Phe-L-Met | 0.2 | L-Ala-L-Phe-L-Met | + |
| L-Tyr-OMe | Gly-Gly-L- | 1.0 | L-Tyr-Gly-Gly-L- | 2.7 |
| | Phe-L-Met | | Phe-L-Met | |
| L-Ala-OMe | Gly-Gly-L- | 0.2 | L-Ala-Gly-Gly-L- | + |
| | Phe-L-Met | | Phe-L-Met | |
| Gly-OMe | Gly-L-Phe | 0.1 | Gly-L-Phe | 17.3 |
| L-Ala-OMe | Gly-L-Phe | 0.1 | L-Ala-Gly-L-Phe | + |
| D-Ala-OMe | Gly-L-Phe | 0.1 | D-Ala-Gly-L-Phe | Tr |

### Example 28

### Isolation of Peptide-forming Enzyme Gene Derived from Empedobacter brevis

Hereinafter, although the following provides a description of the isolation of a peptide-forming enzyme gene, will be explained. As the microbe was used Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002) was used as the microbe. In isolating the gene, Escherichia coli JM-109 was used as a host while pUC118 was used as a vector.

(1) Production of PCR Primer Based on Determined Internal Amino Acid Sequence

A mixed primer having the base sequences indicated in SEQ ID NO.: 3 and SEQ ID NO: 4, respectively, was produced based on the amino acid sequences (SEQ ID NOs: 1 and 2) determined according to the Edman's decomposition method from the a digestion product of lysyl endopeptidase of a peptide-forming enzyme derived from the Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002) digested by a lysyl endopeptidase.

(2) Acquisition of Microbial Cells

Empedobacter brevis strain FERM BP-8113 (Depositary institution: the independent administrative corporation, National Institute of Advanced Industrial Science and Technology, International Patent Organism Depositary, Address of depositary institution: Chuo Dai-6,1 -1 Higashi 1-Chome, Tsukuba-shi, Ibaraki-ken, Japan, International deposit transfer date: Jul. 8, 2002) was cultured at 30° C. for 24 hours on a CM2G agar medium (containing glucose at 50 g/l, yeast extract at 10 g/l, peptone at 10 g/l, sodium chloride at 5 g/l, and agar at 20 g/l, pH 7.0). One loopful of the resulting microbial cells was inoculated into a 500 ml Sakaguchi flask containing 50 ml of a CM2G liquid medium (the aforementioned medium excluding agar) followed by shake culturing at 30° C.

(3) Acquisition of Chromosomal DNA from Microbial Cells

First, 50 ml of culture broth was centrifuged (12,000 rpm, 4° C., 15 minutes) to collect the microbial cells. Then, a chromosomal DNA was acquired from the microbial cells using the QIAGEN Genomic-Tip System (Qiagen) based on the procedure described in the manual therefor.

(4) Acquisition of DNA Fragment Containing Part of Peptide-forming Enzyme Gene by PCR

A DNA fragment containing a portion of the peptide-forming enzyme gene derived from Empedobacter brevis strain FERM BP-8113 (Depositary institution: the independent administrative corporation, National Institute of Advanced Industrial Science and Technology, International Patent Organism Depositary, Address of depositary institution: Dai-6, 1-1 Higashi 1-Chome, Tsukuba-shi, Ibaraki-ken, Japan, International deposit transfer date: Jul. 8, 2002) was acquired by the PCR method using LA-Taq (manufactured by Takara Shuzo). A PCR reaction was then carried out on a chromosomal DNA acquired from Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002) using the primers having the base sequences of SEQ ID NOs: 3 and 4.

The PCR reaction was carried out for 30 cycles under the following conditions using the Takara PCR Thermal Cycler PERSONAL (manufactured by Takara Shuzo).

| | |
|---|---|
| 94° C. | 30 seconds |
| 52° C. | 1 minute |
| 72° C. | 1 minute |

After the reaction, 3 μl of the reaction liquid was applied to 0.8% agarose electrophoresis. As a result, it was verified that a DNA fragment of about 1.5 kilobases (kb) was confirmed to be amplified.

(5) Cloning of Peptide-Forming Enzyme Gene from Gene Library

In order to acquire the entire length of peptide-forming enzyme gene in full-length, Southern hybridization was carried out using the DNA fragment amplified in the PCR procedure as a probe. The procedure for Southern hybridization is explained in Molecular Cloning, 2nd edition, Cold Spring Harbor Press (1989).

The approximately 1.5 kb DNA fragment amplified by the PCR procedure was isolated by 0.8% agarose electrophoresis. The target band was then cut out and the DNA fragment was purified. The DNA fragment was labeled with probe digoxinigen using DIG High Prime (manufactured by Boehringer-Mannheim) based on the procedure described in the manual therefor using DIG High Prime (manufactured by Boehringer-Mannheim).

After completely digesting the chromosomal DNA of Empedobacter brevis acquired in the step (3) of the present Example 28(3) by reacting at 37° C. for 16 hours with restriction enzyme HindII, the resultant DNA was electrophoresed with on 0.8% agarose gel. The electrophoresed chromosomal DNA was blotted onto a positively charged Nylon membrane filter (manufactured by Roche Diagnostics) from the agarose gel after the electrophoresis, followed by treatments consisting of alkaline denaturation, neutralization and immobilization. Hybridization was carried out using EASY HYB (manufactured by Boehringer-Mannheim). After pre-hybridizing the filter at 50° C. for 1 hour, the probe labeled with digoxinigen prepared as described above was added and hybridization was carried out at 50° C. for 16 hours. Subsequently, the filter was washed for 20 minutes at room temperature with 2×SSC containing 0.1% SDS. Moreover, the filter was additionally washed twice at 65° C. for 15 minutes with 0.1×SSC.

Detection of bands that hybridized with the probe was carried out using the DIG Nucleotide Detection Kit (manufactured by Boehringer-Mannheim) based on the procedure described in the manual of the kit. As a result, a roughly 4 kb band was able to be detected that hybridized with the probe.

Then, the chromosomal DNA prepared in the step (3) of the present Example 28(3) was completely digested with HindII. A roughly 4 kb of DNA was separated by 0.8% agarose gel electrophoresis, followed by purification of the DNA using the Gene Clean II Kit (manufactured by Funakoshi) and dissolving the DNA in 10 μl of TE. 4 μl of this product was then mixed with pUC118 HindIII/BAP (manufactured by Takara Shuzo) and a ligation reaction was carried out using the DNA Ligation Kit Ver. 2 (manufactured by Takara Shuzo). 5 μl of the ligation reaction mixture and 100 μl of competent cells of Escherichia coli JM109 (manufactured by Toyobo) were mixed to transform the Escherichia coli. Thus obtained transformants were then applied to a suitable solid medium to produce a chromosomal DNA library.

To acquire the entire full-length of peptide-forming enzyme gene, the chromosomal DNA library was screened by colony hybridization using the aforementioned probe. The procedure for colony hybridization is explained in Molecular Cloning, 2nd edition, Cold Spring Harbor Press (1989).

The colonies of the chromosomal DNA library were transferred to a Nylon membrane filter (Nylon Membrane for Colony and Plaque Hybridization, (manufactured by Roche Diagnostics) followed by treatments consisting of alkali denaturation, neutralization and immobilization. Hybridization was carried out using EASY HYB (manufactured by Boehringer-Mannheim). After pre-hybridizing the filter at 37° C. for 1 hour, the aforementioned probe labeled with digoxinigen was added, followed by hybridization at 50° C. for 16 hours. Subsequently, the filter was washed for 20 minutes at room temperature with 2×SSC containing 0.1% SDS. Moreover, the filter was additionally washed twice at 65° C. for 15 minutes with 0.1×SSC.

Detection of colonies that hybridized with the labeled probe was carried out using the DIG Nucleotide Detection Kit (manufactured by Boehringer-Mannheim) based on the explanation described in the manual of the kit. As a result, two colonies were verified to hybridize with the labeled probe.

(6) Base Sequence of Peptide-Forming Enzyme Gene Derived from Empedobacter brevis

Plasmids possessed by Escherichia coli JM109 were prepared from the aforementioned two colonies that were verified to hybridize with the labeled probe using the Wizard Plus Minipreps DNA Purification System (manufactured by Promega) to and the base sequence of a portion where hybridization with the probe occurred and nearby was determined. The sequencing reaction was carried out using the CEQ DTCS-Quick Start Kit (manufactured by Beckman-Coulter) based on the procedure described in the manual of the kit. In addition, electrophoresis was carried out using the CEQ 2000-XL (manufactured by Beckman-Coulter).

As a result, it was verified that an open reading frame that encodes a protein containing the internal amino acid sequences of the peptide-forming enzyme (SEQ ID NOs: 1 and 2) did exist. Thus, the open reading frame was confirmed to be a gene encoding the peptide-forming enzyme. The base sequence of the full-length of the peptide-forming enzyme genes along with the corresponding amino acid sequences is shown in SEQ ID NO: 5. As a result of analysis on the homology of the resulting open reading frame with the BLASTP program, homology was discovered between the two enzymes; it showed with a homology of 34% as at the amino acid sequence level exhibited with the a-amino acid ester hydrolase of Acetobacter pasteuranus (see Appl. Environ. Microbiol., 68(1), 211-218 (2002), and a homology of 26% at the amino acid sequence level exhibited with the glutaryl-7ACA acylase of Brevibacillus laterosporum (see J. Bacteriol., 173(24), 7848-7855 (1991).

### Example 29

### Expression of Peptide-forming Enzyme Gene Derived from Empedobacter brevis in Escherichia coli

A target gene region on the promoter region of the trp operon on the chromosomal DNA of Escherichia coli W3110 was amplified by carrying out PCR using the oligonucleotides indicated in SEQ ID NOs: 7 and 8 as primers, and the resulting DNA fragments were ligated to a pGEM-Teasy vector (manufactured by Promega). E. coil JM109 was then transformed in this ligation solution, and those strains having the target plasmid in which the direction of the inserted trp promoter is inserted in the opposite to the orientation from of the lac promoter were selected from ampicillin-resistant strains. Next, a DNA fragment containing the trp promoter obtained by treating this plasmid with EcoO1091/EcoRI was ligated to an EcoO109I/EcoRI treatment product of pUC19 (manufactured by Takara). Escherichia coli JM109 was then transformed with this ligation solution and those strains having the target plasmid were selected from ampicillin-resistant strains. Next, a DNA fragment obtained by treating this plasmid with HindIII/PvuII was ligated with to a DNA fragment containing an rrnB terminator obtained by treating pKK223-3 (manufactured by Amersham Pharmacia) with HindIII/HincII. E. coli JM109 was then transformed with this ligation solution, strains having the target plasmid were selected from ampicillin-resistant strains, and the plasmid was designated as pTrpT.

The target gene was amplified by PCR using the chromosomal DNA of Empedobacter brevis strain FERM BP-8113 (Depositary institution: the independent administrative corporation, National Institute of Advanced Industrial Science and Technology, International Patent Organism Depositary, Address of depositary institution: Chuo No Chuo Dai-6, 1-1 Higashi 1-Chome, Tsukuba-shi, Ibaraki-ken, Japan, International deposit transfer date: Jul. 8, 2002) as a template and the oligonucleotides indicated in SEQ ID NO: 9 and 10 as primers. This DNA fragment was then treated with NdeI/PstI, and the resulting DNA fragment was ligated with the NdeI/PstI treatment product of pTrpT. Escherichia coli JM109 was then transformed with this ligation solution, those strains having the target plasmid were selected from ampicillin-resistant strains, and this plasmid was designated as pTrpT_Gtg2.

Escherichia coli JM 109 having pTrpT_Gtg2 was pre-cultured at 30° C. for 24 hours in LB medium containing 100 mg/l of ampicillin. 1 ml of the resulting culture broth was inoculated into a 500 ml Sakaguchi flask containing 50 ml of a medium (D glucose at 2 g/l, yeast extract at 10 g/l, casamino acids at 10 g/l, ammonium sulfate at 5 g/l, potassium dihydrogen phosphate at 3 g/l, dipotassium hydrogen phosphate at 1 g/l, magnesium sulfate heptahydrate at 0.5 g/l, and ampicillin at 100 mg/1), followed by culturing at 25° C. for 24 hours. The culture broth had an L-alanyl-L-glutamine forming activity of 0.44 U per 1 ml of culture broth and it was verified that the cloned gene was expressed by E. coli. Furthermore, no activity was detected for a transformant in which only pTrpT had been introduced as a control.

Prediction of Signal Sequence

When the amino acid sequence of SEQ ID NO: 6 described in the Sequence Listing was analyzed with the Signal P v 1.1 program (see Protein Engineering, Vol. 12, No. 1, pp. 3-9, 1999), it was predicted that amino acids numbers 1 to 22 function as a signal for secretion of peptide into the periplasm, while the mature protein was estimated to be downstream of amino acid number 23.

Verification of Secretion

Escherichia coli JM109, having pTrpT_Gtg2, was pre-cultured at 30° C. for 24 hours in LB medium containing 100 mg/l of ampicillin. 1 ml of the resulting culture broth was inoculated into a 500 ml Sakaguchi flask containing 50 ml of medium (glucose at 2 g/l, yeast extract at 10 g/l, casamino acids at 10 g/l, ammonium sulfate at 5 g/l, potassium dihydrogen phosphate at 3 g/l, dipotassium hydrogen phosphate at 1 g/l, magnesium sulfate heptahydrate at 0.5 g/l, and ampicillin at 100 mg/l), followed by final culturing at 25° C. for 24 hours to obtain microbial cells.

The cultured microbial cells were fractionated into a periplasm fraction and a cytoplasm fraction after disruption of cells by an osmotic pressure shock method using a 20 grams/deciliter (g/dl) sucrose solution. The disrupted microbial cells immersed in the 20 g/dl sucrose solution were immersed in a 5 mM aqueous MgSO₄ solution. The centrifuged supernatant was named a periplasm fraction (“Pe”). In addition, the centrifuged sediment was re-suspended and subjected to ultrasonic crushing. The resultant was named a cytoplasm fraction (“Cy”). The activity of glucose 6-phosphate dehydrogenase, which is known to be present in the cytoplasm, was used as an indicator to verify that the cytoplasm had been separated. This measurement was carried out by adding a suitable amount of enzyme to a reaction solution at 30° C. containing 1 mM glucose 6-phosphate, 0.4 mM NADP, 10 mM MgSO₄, and 50 mM Tris-Cl (pH 8), followed by measurement of absorbance at 340 nm to measure production of NADPH.

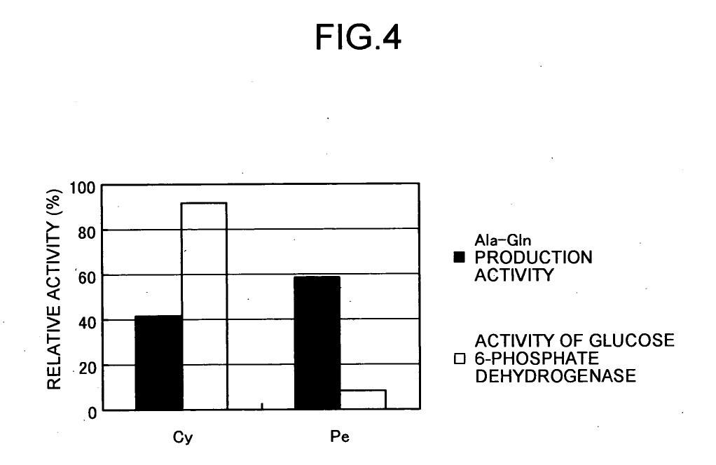

FIG. 4 demonstrates that the amounts of enzymes of in the periplasm fraction and the cytoplasm fraction when the activity of a separately prepared cell-free extract was assigned a value of 100%. The glucose 6-phosphate dehydrogenase activity was not detected in the periplasm fraction. This indicates that the periplasm fraction did not mix in the cytoplasm fraction. About 60% of the Ala-Gln forming activity was recovered in the periplasm fraction, and it was verified that the Ala-Gln forming enzyme was secreted into the periplasm as predicted from the amino acid sequence using the Signal Pv 1.1 program.

### Example 30

### Production of L-Alanyl-L-Glutamine Using Microbial Cells of Sphingobacterium sp.

A 50 ml medium (pH 7.0) containing 5 g of glucose, 5 g of ammonium sulfate, 1 g of monopotassium phosphate, 3 g of dipotassium phosphate, 0.5 g of magnesium sulfate, 10 g of yeast extract, and 10 g of peptone in 1 L was transferred to a 500 mL Sakaguchi flask and sterilized at 11 5° C. for 15 minutes for culturing Sphingobactedum sp. strain FERM BP-8124 (Depositary institution: the independent administrative corporation, National Institute of Advanced Industrial Science and Technology, International Patent Organism Depository, Address of depositary institution: Chuo Dai-6, 1-1 Higashi 1-Chome, Tsukuba-shi, Ibaraki-ken, Japan, International deposit date: Jul. 22, 2002). This was then inoculated with one loopful cells of Sphingobacterium sp. strain FERM BP-8124 (Depositary institution: National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002) cultured at 30° C. for 24 hours in slant agar medium (agar: 20 g/L, pH 7.0) containing 5 g of glucose, 10 g of yeast extract, 10 g of peptone and 5 g of NaCl in 1 L, followed by shake culturing at 30° C. for 20 hours and 120 strokes/minute. 1 ml of this culture broth was then added to the aforementioned medium (50 ml/500 mL Sakaguchi flask) and cultured at 30° C. for 18 hours. After completion of the culture, the microbial cells were separated from the culture broth by centrifugation and suspended in 0.1 M borate buffer (pH 9.0) containing 10 mM EDTA at a concentration of 100 g/L as wet microbial cells. 0.1 mL of 100 mM borate buffer (pH 9.0) containing 10 mM EDTA, 200 mM L-alanine methyl ester hydrochloride and 400 mM L-glutamine was then added to 0.1 mL of this microbial cell suspension. The resulting 0.2 mL of mixture was allowed to react at 25° C. for 120 minutes. The concentration of L-alanyl-L-glutamine produced at this time was 62 mM.

### Example 31

### Purification of Enzyme from Sphingobacterium sp.

The following procedure after centrifugation was carried out either on ice or at 4° C. Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002) was cultured in the same manner as Example 21, and the microbial cells were collected by centrifugation (10,000 rpm, 15 minutes). After washing 2 g of microbial cells with 20 mM Tris-HCl buffer (pH 7.6), they were suspended in 8 ml of the same buffer and subjected to ultrasonic crushing treatment for 45 minutes at 195 W. This ultrasonic crushed suspension was then centrifuged (10,000 rpm, 30 minutes) to remove the crushed cell fragments and obtain a supernatant. This supernatant was dialyzed overnight against 20 mM Tris-HCl buffer (pH 7.6) followed by removal of the insoluble fraction by ultracentrifugation (50,000 rpm, 30 minutes) to obtain a soluble fraction in the form of the supernatant liquid. The resulting soluble fraction was applied to a Q-Sepharose HP column (manufactured by Amersham) pre-equilibrated with Tris-HCl buffer (pH 7.6), and the active fraction was collected from the non-adsorbed fraction. This active fraction was dialyzed overnight against 20 mM acetate buffer (pH 5.0), followed by removal of the insoluble fraction by centrifugation (10,000 rpm, 30 minutes) to obtain a dialyzed fraction in the form of the supernatant liquid. This dialyzed fraction was then applied to an SP-Sepharose HP column (manufactured by Amersham) pre-equilibrated with 20 mM acetate buffer (pH 5.0) to obtain the active fraction in which enzyme was eluted at a linear concentration gradient of the same buffer containing 0 to 1 M NaCl.

### Example 32

### Production of L-Alanyl-L-Glutamine Using Active Fraction

10 μl of the SP-Sepharose HP fraction (about 27 U/ml) purified in Example 31 was added to 90 μl of borate buffer (pH 9.0) containing 111 mM L-alanine methyl ester hydrochloride, 222 mM L-glutamine and 11 mM EDTA, and allowed to react at 25° C. for 120 minutes. As a result, 73 mM of L-alanyl-L-glutamine was produced in the section to which enzyme was added. On the other hand, there was hardly any production of L-Ala-L-Glu observed in the lot to which enzyme was not added, and the amount produced was only about 0.07 mM after reacting for 120 minutes.

### Example 33

### Isolation of Peptide-Forming Enzyme Gene Derived from Sphingobacterium sp.

Although the following provides a description of the isolation of a peptide-forming enzyme gene, Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002) was used as the microbe. Gene isolation was carried out using Escherichia coli DH5a as the host, and pUC1 18 as the vector.

(1) Acquisition of Microbe

Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002) was cultured at 25° C. for 24 hours on CM2G agar medium (containing glucose at 50 g/l, yeast extract at 10 g/l, peptone at 10 g/l, sodium chloride at 5 g/l and agar at 20 g/l, pH 7.0). One loopful of the resulting microbial cells was inoculated into a 500 ml Sakaguchi flask containing 50 ml of CM2G liquid medium (the aforementioned medium excluding agar) followed by shake culturing at 25° C.

(2) Acquisition of Chromosomal DNA from Microbial Cells

50 ml of culture broth was centrifuged (12,000 rpm, 4° C., 15 minutes) to collect the microbial cells. A chromosomal DNA was then acquired from the microbial cells using the Qiagen Genomic-Tip System (Qiagen) therefor.

(3) Acquisition of Probe DNA Fragment by PCR

A DNA fragment containing a portion of the peptide-forming enzyme gene derived from Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit transfer date: Jul. 8, 2002) was acquired by the PCR method using LA-Taq (manufactured by Takara Shuzo). A PCR reaction was then carried out on a chromosomal DNA acquired from Empedobacter brevis strain FERM BP-8113 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki 1-Chome, Japan, International deposit transfer date: Jul. 8, 2002) using primers having the base sequences of SEQ ID NOs: 3 and 4.

The PCR reaction was carried out for 30 cycles under the following conditions using the Takara PCR Thermal Cycler PERSONAL (manufactured by Takara Shuzo).

| | |
|---|---|
| 94° C. | 30 seconds |
| 52° C. | 1 minute |
| 72° C. | 1 minute |

After the reaction, 3 μl of reaction mixture was applied to 0.8% agarose electrophoresis. As a result, a DNA fragment of about 1.5 kb was confirmed to be amplified.

(4) Cloning of Peptide-Forming Enzyme Gene from Gene Library

In order to acquire the entire length of peptide-forming enzyme gene, Southern hybridization was carried out using the DNA fragment amplified in the aforementioned PCR procedure as a probe. The procedure for Southern hybridization is explained in Molecular Cloning, 2nd edition, Cold Spring Harbor Press (1989).

The approximately 1.5 kb DNA fragment amplified by the aforementioned PCR procedure was separated by 0.8% agarose electrophoresis. The target band was then cut out and the DNA fragment was purified. This DNA fragment was labeled with probe digoxinigen using DIG High Prime (manufactured by Boehringer-Mannheim) based on the procedure described in the manual of the kit.

After completely digesting the chromosomal DNA of Sphingobacterium sp. acquired in the step (2) of the present Example 33 by reacting at 37° C. for 16 hours with restriction enzyme Sacl, it was electrophoresed with 0.8% agarose gel. The electrophoresed chromosomal DNA was blotted onto a positively charged Nylon membrane filter (manufactured by Roche Diagnostics) from the agarose gel following electrophoresis followed by treatment consisting of alkaline denaturation, neutralization and immobilization. Hybridization was carried out using EASY HYB (manufactured by Boehringer-Mannheim). After pre-hybridizing the filter at 37° C. for 1 hour, the probe labeled with digoxinigen prepared as described above was added and hybridization was carried out at 37° C. for 16 hours. Subsequently, the filter was washed twice at 60° C. with 1×SSC containing 0.1% SDS.

Detection of bands that hybridized with the probe was carried out using the DIG Nucleotide Detection Kit (manufactured by Boehringer-Mannheim) based on the procedure described in the manual therefor. As a result, a roughly 3 kb band was able to be detected that hybridized with the probe.

The chromosomal DNA prepared in the step (2) of the present Example 33 was completely digested with Sacd. Roughly 3 kb of DNA was separated by 0.8% agarose gel electrophoresis, followed by purification of the DNA using the Gene Clean II Kit (manufactured by Funakoshi) and dissolving in 10 μl of TE. After allowing 4 μl of this product to react with Sacl at 37° C. for 16 hours to completely digest, it was mixed with pUC118 treated with alkaline phosphatase (E. coli C75) at 37° C. for 30 minutes and at 50° C. for 30 minutes, and a ligation reaction was carried out using the DNA Ligation Kit Ver. 2 (manufactured by Takara Shuzo). 5 μl of this ligation reaction liquid and 100 μl of competent cells of Escherichia coli DH5α (manufactured by Takara Shuzo) were mixed to transform the Escherichia col. Thus obtained transformants were then applied to a suitable solid medium to produce a chromosomal DNA library.

In order to acquire the entire length of peptide-forming enzyme gene, the chromosomal DNA library was screened by colony hybridization using the aforementioned probe. The procedure for colony hybridization is explained in Molecular Cloning, 2nd edition, Cold Spring Harbor Press (1989).

The colonies of the chromosomal DNA library were transferred to a Nylon membrane filter—Nylon Membrane for Colony and Plaque Hybridization (manufactured by Roche Diagnostics), followed by treatment consisting of alkaline denaturation, neutralization and immobilization. Hybridization was carried out using EASY HYB (manufactured by Boehringer-Mannheim). After pre-hybridizing the filter at 37° C. for 1 hour, the aforementioned probe labeled with digoxinigen was added followed by hybridizing at 37° C. for 16 hours. Subsequently, the filter was washed twice at 60° C. with 1×SSC containing 1% SDS.

Detection of colonies that hybridized with the labeled probe was carried out using the DIG Nucleotide Detection Kit (manufactured by Boehringer-Mannheim) based on the explanation described in the manual of the kit. As a result, six strains of colonies were confirmed to hybridize with the labeled probe.

(5) Base Sequence of Peptide-Forming Enzyme Gene Derived from Sphingobacterium Sp.

Plasmids possessed by Escherichia coli DH5α were prepared from the aforementioned six strains of microbial cells which were confirmed to hybridize with the labeled probe using the Wizard Plus Minipreps DNA Purification System (manufactured by Promega) to determine the nearby base sequences that hybridized with the probe. The sequencing reaction was carried out using the CEQ DTCS-Quick Start Kit (manufactured by Beckman-Coulter) based on the procedure described in the manual of the kit. In addition, electrophoresis was carried out using the CEQ 2000-XL (manufactured by Beckman-Coulter).

As a result, an open reading frame that encodes peptide-forming enzyme was found to exist. The base sequence of the full-length peptide-forming enzyme gene derived from Sphingobacterium sp. along with the corresponding amino acid sequence is shown in SEQ ID NO: 11. The peptide-forming enzyme derived from Sphingobacterium sp. exhibited homology of 63.5% at the amino acid sequence level with the peptide-forming enzyme derived from the aforementioned Empedobacter brevis (as determined using the BLASTP program).

### Example 34

### Expression of Peptide-Forming Enzyme Gene Derived from Sphingobactedum sp. in Escherchia coli

The target gene was amplified by carrying out PCR using a chromosomal DNA of Sphingobacterium sp. strain FERM BP-8124 (Depositary institution: the independent administrative corporation, National Institute of Advanced Industrial Science and Technology, International Patent Organism Depositary, Address of depositary institution: Chuo Dai-6, 1-1 Higashi 1-Chome, Tsukuba-shi, Ibaraki-ken, Japan, International deposit date: Jul. 22, 2002) as template and the oligonucleotides shown in SEQ ID NOs: 13 and 14 as primers. This DNA fragment was treated with NdeI/XbaI, and the resulting DNA fragment and NdeI/XbaI treatment product of pTrpT were ligated. Escherichia coli JM109 was then transformed with this ligation solution, strains having the target plasmid were selected from ampicillin-resistant strains, and the plasmid was designated as pTrpT_Sm_aet.

Escherichia coli JM109 having pTrpT_Sm_aet was cultured at 25° C. for 20 hours by inoculating one loopful cells of the strain into an ordinary test tube containing 3 ml of medium (glucose at 2 g/l, yeast extract at 10 g/l, casamino acids at 10 g/l, ammonium sulfate at 5 g/l, potassium dihydrogen phosphate at 3 g/l, dipotassium hydrogen phosphate at 1 g/l, magnesium sulfate heptahydrate at 0.5 g/l and ampicillin at 100 mg/I). Cloned gene having L-alanyl-L-glutamine production activity of 2.1 U per 1 ml of culture liquid was confirmed to be expressed by E. coli. Furthermore, activity was not detected for a transformant containing only pTrpT used as a control.

Prediction of Signal Sequence

When the amino acid sequence of SEQ ID NO: 12 described in the Sequence Listing was analyzed with the Signal Pv1.1 program (see Protein Engineering, Vol.12, No. 1, pp. 3-9, 1999), it was predicted that amino acids numbers 1 to 20 function as a signal for secretion of peptide into the periplasm, while the mature protein was estimated to be downstream of amino acid number 21.

Confirmation of Signal Sequence

Escherichia coli JM1 09, having pTrpT_Sm_aet, was cultured at 25° C. for 20 hours by inoculating one loopful cells of the strain into an ordinary test tube containing 50 ml of medium (glucose at 2 g/l, yeast extract at 10 g/l, casamino acids at 10 g/l, ammonium sulfate at 5 g/l, potassium dihydrogen phosphate at 3 g/l, dipotassium hydrogen phosphate at 1 g/l, magnesium sulfate heptahydrate at 0.5 g/l and ampicillin at 100 mg/l).

The following procedure after centrifugation was carried out either on ice or at 4° C. Following completion of culturing, the microbial cells were separated from the culture broth by centrifugation, and after washing with 100 mM phosphate buffer (pH 7), were suspended in the same buffer. The microbial cells were then subjected to ultrasonic crushing for 20 minutes at 195 W, the ultrasonic crushed suspension was centrifuged (12,000 rpm, 30 minutes) to remove the crushed cell fragments and obtain a soluble fraction. The resulting soluble fraction was applied to a CHT-II column (manufactured by Biorad) pre-equilibrated with 100 mM phosphate buffer (pH 7), and enzyme was eluted at a linear concentration gradient by 500 mM phosphate buffer. A solution obtained by mixing the active fraction with a 5-fold volume of 2 M ammonium sulfate and 100 mM phosphate buffer was applied to a Resource-PHE column (Amersham) pre-equilibrated with 2 M ammonium sulfate and 100 mM phosphate buffer, and enzyme was eluted at a linear concentration gradient by 2 to 0 M ammonium sulfate to obtain an active fraction solution. As a result of these procedures, the peptide-forming enzyme was confirmed to be uniformly purified in terms of electrophoresis.

When the amino acid sequence of the aforementioned peptide-forming enzyme was determined by Edman's decomposition method, the amino acid sequence of SEQ ID NO: 15 was acquired, and the mature protein was confirmed to be downstream from amino acid number 21 as was predicted by the SignalP v 1.1 program.

### Example 35

### Isolation of Peptide-forming Enzyme Gene Derived from Pedobacter heparinus IFO 12017

Hereinafter, the isolation of a peptide-forming enzyme gene will be described. The microbe used is Pedobacter heparinus strain IFO 12017 (Depositary institution: Institute of Fermentation, Address of depositary institution: 2-17-85 Jusanbon-cho, Yodogawa-ku, Osaka-shi, Japan). Escherichia coli JM-109 was used as a host while pUC118 was used as a vector in isolating the gene.

(1) Acquisition of Microbe

Pedobacter heparinus strain IFO-1 2017 (Depositary institution: Institute of Fermentation, Address of depositary institution: 2-17-85 Jusanbon-cho, Yodogawa-ku, Osaka-shi, Japan) was cultured at 25° C. for 24 hours on CM2G agar medium (containing glucose at 50 g/l, yeast extract at 10 g/l, peptone at 10 g/l, sodium chloride at 5 g/l and agar at 20 g/l, pH 7.0). One loopful of the resulting microbial cells were inoculated into a 500 ml Sakaguchi flask containing 50 ml of CM2G liquid medium (the aforementioned medium excluding agar) followed by shake culturing at 25° C.

(2) Acquisition of Chromosomal DNA from Microbial Cells

50 ml of culture broth was centrifuged (12,000 rpm, 4° C., 15 minutes) to collect the microbial cells. A chromosomal DNA was then acquired from the microbial cells using the Qiagen Genomic-Tip System (Qiagen) based on the procedure described in the manual therefor.

(3) Acquisition of Probe DNA Fragment by PCR

A DNA fragment containing a portion of the peptide-forming enzyme gene derived from Pedobacter heparinus strain IFO-12017 (Depositary institution: Institute of Fermentation, Address of depositary institution: 2-17-85 Jusanbon-cho, Yodogawa-ku, Osaka-shi, Japan) was acquired by the PCR method using LA-Taq (manufactured by Takara Shuzo). A PCR reaction was then carried out on a chromosomal DNA acquired from Pedobacter heparinus strain IFO-12017 (Depositary institution: Institute of Fermentation, Address of depositary institution: 2-17-85 Jusanbon-cho, Yodogawa-ku, Osaka-shi, Japan) using primers having the base sequences of SEQ ID NOs: 15 and 16. A DNA fragment of about 1 kb amplified by PCR was separated by 0.8% agarose electrophoresis. The target band was then cut out and thus obtained DNA fragment was purified. This DNA fragment was labeled with probe digoxinigen using DIG High Prime based on the procedure described in the manual (manufactured by Boehringer-Mannheim).

(4) Cloning of Peptide-Forming Enzyme Gene from Gene Library

To acquire the full-length peptide-forming enzyme gene, Southern hybridization was carried out using the DNA fragment amplified in the aforementioned PCR procedure as a probe. The procedure for Southern hybridization is explained in Molecular Cloning, 2nd edition, Cold Spring Harbor Press (1989).

After completely digesting the chromosomal DNA of Pedobacter heparinus strain IFO-12017 (Depositary institution: Institute of Fermentation, Address of depositary institution: 2-17-85 Jusanbon-cho, Yodogawa-ku, Osaka-shi, Japan) by reacting at 37° C. for 16 hours with restriction enzyme HindII, it was electrophoresed with 0.8% agarose gel. The electrophoresed chromosomal DNA was blotted onto a positively charged Nylon membrane filter (manufactured by Roche Diagnostics) from the agarose gel after the electrophoresis, followed by treatment consisting of alkali denaturation, neutralization, and immobilization. Hybridization was carried out using EASY HYB (manufactured by Boehringer-Mannheim). After pre-hybridizing the filter at 50° C. for 1 hour, the probe labeled with digoxinigen prepared as described above was added and hybridization was carried out at 50° C. for 16 hours. Subsequently, the filter was washed twice at 60° C. with 1×SSC containing 0.1% SDS.

Detection of bands that hybridized with the probe was carried out based on the procedure described in the manual using the DIG Nucleotide Detection Kit (manufactured by Boehringer-Mannheim). As a result, a roughly 5 kb band was able to be detected that hybridized with the probe.

The chromosomal DNA of Pedobacter heparinus strain IFO-12017 (Depositary institution: Institute of Fermentation, Address of depositary institution: 2-17-85 Jusanbon-cho, Yodogawa-ku, Osaka-shi, Japan) was completely digested with HindIII. Roughly 5 kb of DNA were separated by 0.8% agarose gel electrophoresis followed by purification of the DNA using the Gene Clean II Kit (manufactured by Funakoshi) and dissolving in 10 μl of TE. 4 μl of this product was then mixed with pUC118 HindIII/BAP (manufactured by Takara Shuzo) and a ligation reaction was carried out using the DNA Ligation Kit Ver. 2 (manufactured by Takara Shuzo). 5 μl of this ligation reaction liquid and 100 μl of competent cells of Escherichia coli JM109 (manufactured by Takara Shuzo) were mixed to transform the Escherichia coli. The obtained transformants were then applied to a suitable solid medium to produce a chromosomal DNA library.

In order to acquire the full-length peptide-forming enzyme gene, the chromosomal DNA library was screened by colony hybridization using the aforementioned probe. The procedure for colony hybridization is explained in Molecular Cloning, 2nd edition, Cold Spring Harbor Press (1989).

The colonies of the chromosomal DNA library were transferred to a Nylon membrane filter, Nylon Membrane for Colony and Plaque Hybridization, (manufactured by Roche Diagnostics), followed by treatment consisting of alkali denaturation, neutralization and immobilization. Hybridization was carried out using EASY HYB (manufactured by Boehringer-Mannheim). After pre-hybridizing the filter at 37° C. for 1 hour, the aforementioned probe labeled with digoxinigen was added followed by hybridizing at 37° C. for 16 hours. Subsequently, the filter was washed twice at 60° C. with 1×SSC containing 1% SDS.

Detection of colonies that hybridized with the labeled probe was carried out based on the explanation described in the manual using the DIG Nucleotide Detection Kit (manufactured by Boehringer-Mannheim). As a result, 1 strain of colonies was confirmed to hybridize with the labeled probe.

(5) Base Sequence of Peptide-forming Enzyme Gene Derived from Pedobacter heparinus Strain IFO-12017

Plasmids retained by Escherichia coli JM109 were prepared from the aforementioned strain of microbial cells which were confirmed to hybridize with the labeled probe, and the nearby base sequence that hybridized with the probe was determined. The sequencing reaction was carried out using the CEQ DTCS-Quick Start Kit (manufactured by Beckman-Coulter) based on the procedure described in the manual. In addition, electrophoresis was carried out using the CEQ 2000-XL (Beckman-Coulter).

As a result, an open reading frame that encodes peptide-forming enzyme was found to exist. The base sequence of the full-length peptide-forming enzyme gene derived from Pedobacter heparinus strain IFO-12017 (Depositary institution: Institute of Fermentation, Address of depositary institution: 2-17-85 Jusanbon-cho, Yodogawa-ku, Osaka-shi, Japan), along with the corresponding amino acid sequence, is shown in SEQ ID NO: 17 of the Sequence Listing.

### Example 36

### Expression of Peptide-Forming Enzyme Gene Derived from Pedobacter Heparinus strain IFO-12017 in E. coli

The target gene was amplified by carrying out PCR using a chromosomal DNA of Pedobacter heparinus strain IFO-12017 (Depositary institution: Institute of Fermentation, Osaka, Address of depositary institution: 2-17-85 Jusanbon-cho, Yodogawa-ku, Osaka-shi, Japan) as template and the oligonucleotides shown in SEQ ID NOs: 19 and 20 as primers. This DNA fragment was treated with NdeI/HindIII, and the resulting DNA fragment and NdeI/HindIII treatment product of pTrpT were ligated. Escherichia coli JM109 was then transformed with this ligation solution, strains having the target plasmid were selected from ampicillin-resistant strains, and the plasmid was designated as pTrpT_Ph_aet.

Escherichia coli JM1 09 having pTrpT_Ph_aet was cultured at 25° C. for 20 hours by inoculating one loopful cells of the strain into an ordinary test tube containing 3 ml of medium (glucose at 2 g/l, yeast extract at 10 g/l, casamino acids at 10 g/l, ammonium sulfate at 5 g/l, potassium dihydrogen phosphate at 3 g/l, dipotassium hydrogen phosphate at 1 g/l, magnesium sulfate heptahydrate at 0.5 g/l and ampicillin at 100 mg/I). A cloned gene having L-alanyl-L-glutamine production activity of 0.3 U per ml of culture liquid was confirmed to be expressed in E. coli. Furthermore, no activity was detected for a transformant containing only pTrpT used as a control.

### Example 37

### Isolation of Peptide-Forming Enzyme Gene Derived from Taxeobacter Gelupurpurascens Strain DSMZ 11116

Hereinafter, the isolation of peptide-forming enzyme gene will be described. The microbe used is Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1b, 38124 Braunschweig, Germany) was used for the microbe. Escherichia coli JM-109 was used as a host while pUC118 was used as a vector in isolating the gene.

(1) Microbe Acquisition

Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1b, 38124 Braunschweig, Germany) was cultured at 25° C. for 24 hours on CM2G agar medium (containing glucose at 50 g/l, yeast extract at 10 g/l, peptone at 10 g/l, sodium chloride at 5 g/l and agar at 20 g/l, pH 7.0). One loopful of the resulting microbial cells were inoculated into a 500 ml Sakaguchi flask containing 50 ml of CM2G liquid medium (the aforementioned medium excluding agar) followed by shake culturing at 25° C.

(2) Acquisition of Chromosomal DNA from Microbial Cells

50 ml of culture liquid were centrifuged (12,000 rpm, 4° C., 15 minutes) to collect the microbial cells. A chromosomal DNA was then acquired from the microbial cells using the Qiagen Genomic-Tip System (Qiagen) based on the procedure described in the manual therefor.

(3) Acquisition of Probe DNA Fragment by PCR

A DNA fragment containing a portion of the peptide-forming enzyme gene derived from Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1 b, 38124 Braunschweig, Germany) was acquired by the PCR method using LA-Taq (manufactured by Takara Shuzo). A PCR reaction was then carried out on a chromosomal DNA acquired from Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1b, 38124 Braunschweig, Germany) using primers having the base sequences of SEQ ID NOs: 21 and 16. A DNA fragment of about 1 kb amplified by PCR was separated by 0.8% agarose electrophoresis. The target band was then cut out and purified. This DNA fragment was labeled with probe digoxinigen using DIG High Prime (manufactured by Boehringer-Mannheim) based on the procedure described in the manual.

(4) Cloning of Peptide-Forming Enzyme Gene from Gene Library

To acquire the full-length peptide-forming enzyme gene, Southern hybridization was carried out using the DNA fragment amplified in the aforementioned PCR procedure as a probe. The procedure for Southern hybridization is explained in Molecular Cloning, 2nd edition, Cold Spring Harbor Press (1989).

After completely digesting the chromosomal DNA of Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1b, 38124 Braunschweig, Germany) by reacting at 37° C. for 16 hours with restriction enzyme PstI, it was electrophoresed with 0.8% agarose gel. The electrophoresed chromosomal DNA was blotted onto a positively charged Nylon membrane filter (manufactured by Roche Diagnostics) from the agarose gel following electrophoresis followed by treatment consisting of alkali denaturation, neutralization and immobilization. Hybridization was carried out using EASY HYB (manufactured by Boehringer-Mannheim). After pre-hybridizing the filter at 50° C. for 1 hour, the probe labeled with digoxinigen prepared as described above was added and hybridization was carried out at 50° C. for 16 hours. Subsequently, the filter was washed twice at 60° C. with 1×SSC containing 0.1% SDS.

Detection of bands that hybridized with the probe was carried out based on the procedure described in the manual using the DIG Nucleotide Detection Kit (manufactured by Boehringer-Mannheim). As a result, a roughly 5 kb band was able to be detected that hybridized with the probe.

The chromosomal DNA of Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1b, 38124 Braunschweig, Germany) was completely digested with HindIII. Roughly 5 kb of DNA were separated by 0.8% agarose gel electrophoresis followed by purification of the DNA using the Gene Clean II Kit (manufactured by Funakoshi) and dissolving in 10 μl of TE. 4 μl of this product were then mixed with pUC118 PstI/BAP (manufactured by Takara Shuzo) and a ligation reaction was carried out using the DNA Ligation Kit Ver. 2 (manufactured by Takara Shuzo). 5 μl of this ligation reaction liquid and 100 μl of competent cells of Escherichia coli JM1 09 (manufactured by Takara Shuzo) were mixed to transform the Escherichia coli. Thus obtained transformants were then applied to a suitable solid medium to produce a chromosomal DNA library.

In order to acquire the entire length of peptide-forming enzyme gene, the chromosomal DNA library was screened by colony hybridization using the aforementioned probe. The procedure for colony hybridization is explained in Molecular Cloning, 2nd edition, Cold Spring Harbor Press (1989).

The colonies of the chromosomal DNA library were transferred to a Nylon membrane filter, Nylon Membrane for Colony and Plaque Hybridization, (manufactured by Roche Diagnostics) followed by treatment consisting of alkaline denaturation, neutralization and immobilization. Hybridization was carried out using EASY HYB (manufactured by Boehringer-Mannheim). After pre-hybridizing the filter at 37° C. for 1 hour, the aforementioned probe labeled with digoxinigen was added followed by hybridizing at 37° C. for 16 hours. Subsequently, the filter was washed twice at 60° C. with 1×SSC containing 0.1% SDS.

Detection of colonies that hybridized with the labeled probe was carried out based on the manual using the DIG Nucleotide Detection Kit (manufactured by Boehringer-Mannheim). As a result, 1 strain of colonies was confirmed to hybridize with the labeled probe.

(5) Base Sequence of Peptide-forming Enzyme Gene Derived from Taxeobacter gelupurpurascens Strain DSMZ 11116

Plasmids retained by Escherichia coli JM109 were prepared from the aforementioned strain of microbial cells which were confirmed to hybridize with the labeled probe, and the nearby base sequence that hybridized with the probe was determined. The sequencing reaction was carried out using the CEQ DTCS-Quick Start Kit (manufactured by Beckman-Coulter) based on the procedure described in the manual. In addition, electrophoresis was carried out using the CEQ 2000-XL (manufactured by Beckman-Coulter).

As a result, an open reading frame that encodes peptide-forming enzyme was found to exist. The base sequence of the entire length of the peptide-forming enzyme gene derived from Taxeobacter gelupurpurascens strain DSMZ 11116 (Depositary institution: Deutche Sammlung von Mikroorganismen und Zellkulturen GmbH (German Collection of Microbes and Cell Cultures), Address of depositary institution: Mascheroder Weg 1b, 38124 Braunschweig, Germany), along with the corresponding amino acid sequence, are shown in SEQ ID NO: 22 of the Sequence Listing.

### Example 38

### Isolation of Peptide-Forming Enzyme Gene Derived from Cyclobacterium Marinum Strain ATCC 25205

Hereinafter, the isolation of peptide-forming enzyme gene will be described. The microbe used is Cyclobacterium marinum strain ATCC 25205 (Depositary institution: American Type Culture Collection, Address of depositary institution: P.O. Box 1549, Manassas, Va. 20110, the United States of America). Escherichia coli JM-109 was used as a host while pUC118 was used for the vector in isolating the gene.

(1) Microbe Acquisition

Cyclobacterium marinum strain ATCC 25205 (Depositary institution: American Type Culture Collection, Address of depositary institution: P.O. Box 1549, Manassas, Va. 20110, the United States of America) was cultured at 25° C. for 24 hours on CM2G agar medium (containing glucose at 50 g/l, yeast extract at 10 g/l, peptone at 10 g/l, sodium chloride at 5 g/l and agar at 20 g/l, pH 7.0). One loopful of the resulting microbial cells was inoculated into a 500 ml Sakaguchi flask containing 50 ml of CM2G liquid medium (the aforementioned medium excluding agar), followed by shake culturing at 25° C.

(2) Acquisition of Chromosomal DNA from Microbial Cells

50 ml of culture broth were centrifuged (12,000 rpm, 4° C., 15 minutes) to collect the microbial cells. A chromosomal DNA was then acquired from the microbial cells based on the procedure described in the manual using the Qiagen Genomic-Tip System (Qiagen).

(3) Acquisition of Probe DNA Fragment by PCR

A DNA fragment containing a portion of the peptide-forming enzyme gene derived from Cyclobacterium marinum strain ATCC 25205 (Depositary institution: American Type Culture Collection, Address of depositary institution: P.O. Box 1549, Manassas, Va. 20110, the United States of America) was acquired by the PCR method using LA-Taq (manufactured by Takara Shuzo). A PCR reaction was then carried out on a chromosomal DNA acquired from Cyclobacterium marinum strain ATCC 25205 (Depositary institution: American Type Culture Collection, Address of depositary institution: P.O. Box 1549, Manassas, Va. 20110, the United States of America) using primers having the base sequences of SEQ ID NOs: 15 and 16. A DNA fragment of about 1 kb amplified by PCR was separated by 0.8% agarose electrophoresis. The target band was then cut out and the DNA fragment was purified. This DNA fragment was labeled with probe digoxinigen based on the procedure described in the manual using DIG High Prime (manufactured by Boehringer-Mannheim).

(4) Cloning of Peptide-Forming Enzyme Gene from Gene Library

In order to acquire the full-length peptide-forming enzyme gene, Southern hybridization was first carried out using the DNA fragment amplified in the aforementioned PCR procedure as a probe. The procedure for Southern hybridization is explained in Molecular Cloning, 2nd edition, Cold Spring Harbor Press (1989).

After completely digesting the chromosomal DNA of Cyclobacterium marinum strain ATCC 25205 (Depositary institution: American Type Culture Collection, Address of depositary institution: P.O. Box 1549, Manassas, Va. 20110, the United States of America) by reacting at 37° C. for 16 hours with restriction enzyme HincII, each was electrophoresed with 0.8% agarose gel. The electrophoresed chromosomal DNA was blotted onto a positively charged Nylon membrane filter (manufactured by Roche Diagnostics) from the agarose gel following electrophoresis followed by treatment consisting of alkali denaturation, neutralization and immobilization. Hybridization was carried out using EASY HYB (manufactured Boehringer-Mannheim). After pre-hybridizing the filter at 50° C. for 1 hour, the probe labeled with digoxinigen prepared as described above was added and hybridization was carried out at 50° C. for 16 hours. Subsequently, the filter was washed twice at 60° C. with 1×SSC containing 0.1% SDS.

Detection of bands that hybridized with the probe was carried out based on the procedure described in the manual using the DIG Nucleotide Detection Kit (manufactured by Boehringer-Mannheim). As a result, a roughly 7k band was able to be detected that hybridized with the probe in the PstI digestion product, while a 2k band was able to be detected that hybridized with the probe in the HincII digestion product.

The chromosomal DNA of Cyclobacterium marinum strain ATCC 25205 (Depositary institution: American Type Culture Collection, Address of depositary institution: P.O. Box 1549, Manassas, Va. 20110, the United States of America) was completely digested with PstI or HincII. Roughly 7 kb or 2 kb of DNA were respectively separated by 0.8% agarose gel electrophoresis, followed by purification of the DNA using the Gene Clean II Kit (Funakoshi) and dissolving in 10 μl of TE. 4 μl of this product were then mixed with pUC118 PstI/BAP (manufactured by Takara Shuzo) or pUC118 HincII/BAP (manufactured by Takara Shuzo) and a ligation reaction was carried out using the DNA Ligation Kit Ver. 2 (manufactured by Takara Shuzo). 5 μl of this ligation reaction liquid and 100 μl of competent cells of Escherichia coli JM109 (manufactured by Takara Shuzo) were respectively mixed to transform the Escherichia coli. Thus obtained transformants were then applied to a suitable solid medium to produce a chromosomal DNA library.

To acquire the full-length peptide-forming enzyme gene, the chromosomal DNA library was screened by colony hybridization using the aforementioned probe. The procedure for colony hybridization is explained in Molecular Cloning, 2nd edition, Cold Spring Harbor Press (1989).

The colonies of the chromosomal DNA library were transferred to a Nylon membrane filter, Nylon Membrane for Colony and Plaque Hybridization, (manufactured by Roche Diagnostics), followed by treatment consisting of alkali denaturation, neutralization, and immobilization. Hybridization was carried out using EASY HYB (manufactured by Boehringer-Mannheim). After pre-hybridizing the filter at 37° C. for 1 hour, the aforementioned probe labeled with digoxinigen was added followed by hybridizing at 37° C. for 16 hours. Subsequently, the filter was washed twice at 60° C. with 1×SSC containing 0.1% SDS.

Detection of colonies that hybridized with the labeled probe was carried out based on the manual using the DIG Nucleotide Detection Kit (manufactured by Boehringer-Mannheim). As a result, 1 strain of colonies each was confirmed to hybridize with the labeled probe.

(5) Base Sequence of Peptide-Forming Enzyme Gene Derived from Cyclobacterium marinum Strain ATCC 25205

Plasmids retained by Escherichia coli JM109 were prepared from various aforementioned strains of microbial cells which were confirmed to hybridize with the labeled probe, and the nearby base sequence that hybridized with the probe was determined. The sequencing reaction was carried out using the CEQ DTCS-Quick Start Kit (manufactured by Beckman-Coulter) based on the procedure described in the manual therefor. In addition, electrophoresis was carried out using the CEQ 2000-XL (manufactured by Beckman-Coulter).

As a result, an open reading frame that encodes peptide-forming enzyme was found to exist. The base sequence of the full-length peptide-forming enzyme gene derived from Cyclobacterium marinum strain ATCC 25205 (Depositary institution: American Type Culture Collection, Address of depositary institution: P.O. Box 1549, Manassas, Va. 20110, the United States of America), along with the corresponding amino acid sequence, is shown in SEQ ID NO: 24 of the Sequence Listing.

### Example 39

### Isolation of Peptide-Forming Enzyme Gene Derived from Psycloserpens Burtonensis Strain ATCC 700359

Hereinafter, the isolation of a peptide-forming enzyme gene will be explained. The microbe used is Psycloserpens burtonensis strain ATCC 700359 (Depositary institution: American Type Culture Collection, Address of depositary institution: P.O. Box 1549, Manassas, Va. 20110, the United States of America). Escherichia coli JM-109 was used for the host while pUC118 was used for the vector in isolating the gene.

(1) Acquisition of Microbe

Psycloserpens burtonensis strain ATCC 700359 (Depositary institution: American Type Culture Collection, Address of depositary institution: P.O. Box 1549, Manassas, Va. 20110, the United States of America) was cultured at 10° C. for 24 hours on CM2G agar medium (containing glucose at 50 g/l, yeast extract at 10 g/l, peptone at 10 g/l, sodium chloride at 5 g/l and agar at 20 g/l, pH 7.0). One loopful of the resulting microbial cells was inoculated into a 500 ml Sakaguchi flask containing 50 ml of CM2G liquid medium (the aforementioned medium excluding agar) followed by shake culturing at 10° C.

(2) Acquisition of Chromosomal DNA from Microbial Cells

50 ml of culture liquid were centrifuged (12,000 rpm, 4° C., 15 minutes) to collect the microbial cells. A chromosomal DNA was then acquired from the microbial cells using the Qiagen Genomic-Tip System (Qiagen) based on the procedure described in the manual therefor.

(3) Acquisition of Probe DNA Fragment by PCR

A DNA fragment containing a portion of the peptide-forming enzyme gene derived from Psycloserpens burtonensis strain ATCC 700359 (Depositary institution: American Type Culture Collection, Address of depositary institution: P.O. Box 1549, Manassas, Va. 20110, the United States of America) was acquired by the PCR method using LA-Taq (manufactured by Takara Shuzo). A PCR reaction was then carried out on a chromosomal DNA acquired from Psycloserpens burtonensis strain ATCC 700359 (Depositary institution: American Type Culture Collection, Address of depositary institution: P.O. Box 1549, Manassas, Va. 201 10, the United States of America) using primers having the base sequences of SEQ ID NOs: 15 and 16. A DNA fragment of about 1 kb amplified by PCR was separated by 0.8% agarose electrophoresis. The target band was then cut out and the DNA fragment was purified. This DNA fragment was labeled with probe digoxinigen based on the procedure described in the manual using DIG High Prime (manufactured by Boehringer-Mannheim).

(4) Cloning of Peptide-Forming Enzyme Gene from Gene Library

In order to acquire the entire length of peptide-forming enzyme gene, Southern hybridization was carried out using the DNA fragment amplified in the aforementioned PCR procedure as a probe. The procedure for Southern hybridization is explained in Molecular Cloning, 2nd edition, Cold Spring Harbor Press (1989).

After completely digesting the chromosomal DNA of Psycloserpens burtonensis strain ATCC 700359 (Depositary institution: American Type Culture Collection, Address of depositary institution: P.O. Box 1549, Manassas, Va. 201 10, the United States of America) by reacting at 37° C. for 16 hours with restriction enzyme EcoRI, it was electrophoresed with 0.8% agarose gel. The electrophoresed chromosomal DNA was blotted onto a positively charged Nylon membrane filter (manufactured by Roche Diagnostics) from the agarose gel following electrophoresis followed by treatment consisting of alkaline denaturation, neutralization and immobilization. Hybridization was carried out using EASY HYB (manufactured by Boehringer-Mannheim). After pre-hybridizing the filter at 50° C. for 1 hour, the probe labeled with digoxinigen prepared as described above was added and hybridization was carried out at 50° C. for 16 hours. Subsequently, the filter was washed twice at 60° C. with 1×SSC containing 0.1% SDS.

Detection of bands that hybridized with the probe was carried out using the DIG Nucleotide Detection Kit (manufactured by Boehringer-Mannheim) based on the procedure described in the manual therefor. As a result, a roughly 7 kb band was able to be detected that hybridized with the probe.

The chromosomal DNA of Psycloserpens burtonensis strain ATCC 700359 (Depositary institution: American Type Culture Collection, Address of depositary institution: P.O. Box 1549, Manassas, Va. 201 10, the United States of America) was completely digested with EcoRI. Roughly 7 kb of DNA were separated by 0.8% agarose gel electrophoresis followed by purification of the DNA using the Gene Clean II Kit (manufactured by Funakoshi) and dissolving in 10 μl of TE. 4 l of this product were then mixed with pUC118 EcoRI/BAP (manufactured by Takara Shuzo) and a ligation reaction was carried out using the DNA Ligation Kit Ver. 2 (manufactured by Takara Shuzo). 5 μμl of this ligation reaction liquid and 100 μl of competent cells of Escherichia coli JM109 (manufactured by Takara Shuzo) were mixed to transform the Escherichia coli. Thus obtained transformants were then applied to a suitable solid medium to produce a chromosomal DNA library.

To acquire the full-length peptide-forming enzyme gene, the chromosomal DNA library was screened by colony hybridization using the aforementioned probe. The procedure for colony hybridization is explained in Molecular Cloning, 2nd edition, Cold Spring Harbor Press (1989).

The colonies of the chromosomal DNA library were transferred to a Nylon membrane filter, Nylon Membrane for Colony and Plaque Hybridization, (manufactured by Roche Diagnostics), followed by treatment consisting of alkali denaturation, neutralization, and immobilization. Hybridization was carried out using EASY HYB (manufactured by Boehringer-Mannheim). After pre-hybridizing the filter at 37° C. for 1 hour, the aforementioned probe labeled with digoxinigen was added followed by hybridizing at 37° C. for 16 hours. Subsequently, the filter was washed twice at 60° C. with 1×SSC containing 0.1% SDS.

Detection of colonies that hybridized with the labeled probe was carried out based on the manual using the DIG Nucleotide Detection Kit (manufactured by Boehringer-Mannheim). As a result, 1 strain of colonies was confirmed to hybridize with the labeled probe.

(5) Base Sequence of Peptide-Forming Enzyme Gene Derived from Psycloserpens burtonensis Strain ATCC 700359

Plasmids retained by Escherichia coli JM109 were prepared from the aforementioned strain of microbial cells which were confirmed to hybridize with the labeled probe, and the nearby base sequence that hybridized with the probe was determined. The sequencing reaction was carried out using the CEQ DTCS-Quick Start Kit (manufactured by Beckman-Coulter) based on the procedure described in the manual. In addition, electrophoresis was carried out using the CEQ 2000-XL (manufactured by Beckman-Coulter).

As a result, an open reading frame that encodes peptide-forming enzyme was found to exist. The base sequence of the full-length peptide-forming enzyme gene derived from Psycloserpens burtonensis strain ATCC 700359 (Depositary institution: American Type Culture Collection, Address of depositary institution: P.O. Box 1549, Manassas, Va 201 10, the United States of America), along with the corresponding amino acid sequence, are shown in SEQ ID NO: 31 of the Sequence Listing.

Although the invention has been described with respect to a specific embodiment for a complete and clear disclosure, the appended claims are not to be thus limited but are to be construed as embodying all modifications and alternative constructions that may occur to one skilled in the art which fairly fall within the basic teaching herein set forth.

Sequence Listing

SEQ ID NO: 3: Synthetic primer 1

SEQ ID NO: 4: Synthetic primer 2

SEQ ID NO: 5: Gene encoding a peptide-forming enzyme

SEQ ID NO: 7: Synthetic primer for preparing pTrpT

SEQ ID NO: 8: Synthetic primer for preparing pTrpT

SEQ ID NO: 9: Synthetic primer for preparing pTrpT_Gtg2

SEQ ID NO: 10: Synthetic primer for preparing pTrpT_Gtg2

SEQ ID NO: 11: Gene encoding peptide-forming enzyme

SEQ ID NO: 13: Synthetic primer for preparing pTrpT_Sm_aet

SEQ ID NO: 14: Synthetic primer for preparing pTrpT_Sm_aet

SEQ ID NO: 15: Mix primer 1 for Aet

SEQ ID NO: 16: Mix primer 2 for Aet

SEQ ID NO: 19: Primer 1 for constructing aet expression vectors derived from Pedobacter

SEQ ID NO: 20: Primer 2 for constructing aet expression vectors derived from Pedobacter.

SEQ ID NO: 21: Mix primer 3 for Aet

What is claimed is:

1. A DNA encoding a protein selected from the group consisting of (A), (C), (E), (G), (I), (K), (M), (O), (Q), (S), (U), and (W), wherein said protein has an amino acid sequence defined as follows:
(A) an amino acid sequence consisting of amino acid residue numbers 23 to 616 of SEQ ID NO: 6,
(C) an amino acid sequence consisting of amino acid residue numbers 21 to 619 of SEQ ID NO: 12,
(E) an amino acid sequence consisting of amino acid residue numbers 23 to 625 of SEQ ID NO: 18,
(G) an amino acid sequence consisting of amino acid residue numbers 23 to 645 of SEQ ID NO: 23,
(I) an amino acid sequence consisting of amino acid residue numbers 26 to 620 of SEQ ID NO: 25,
(K) an amino acid sequence consisting of amino acid residue numbers 18 to 644 of SEQ ID NO: 27,
(M) an amino acid sequence consisting of SEQ ID NO: 6,
(O) an amino acid sequence consisting of SEQ ID NO: 12,
(Q) an amino acid sequence consisting of SEQ ID NO: 18,
(S) an amino acid sequence consisting of SEQ ID NO: 23,
(U) an amino acid sequence consisting of SEQ ID NO: 25, or
(W) an amino acid sequence consisting of SEQ ID NO: 27.

2. A recombinant DNA comprising the DNA according to claim 1.

3. A transformed cell comprising the recombinant DNA according to claim 2.

4. A method for producing a peptide-forming enzyme comprising:
culturing the transformed cell according to claim 3 in a medium for a time and under conditions suitable to produce the peptide-forming enzyme, and
accumulating the peptide-forming enzyme in the medium and/or transformed cell.

5. A method for producing a dipeptide comprising:
culturing the transformed cell according to claim 3 in a medium for a time and under conditions suitable to produce a peptide-forming enzyme in a culture, and
mixing the culture with a carboxy component and an amine component to synthesize a dipeptide by enzymatic catalysis facilitated by a peptide-forming enzyme encoded by said DNA.

6. The method for producing a dipeptide according to claim 5, wherein said cell is a microbe belonging to the genus Sphingobacterium that has an ability to form the dipeptide from the carboxy component and the amine component.

7. The method for producing a dipeptide according to claim 6, wherein said cell is separated from said culture.

8. The method for producing a dipeptide according to claim 6, wherein said cell is a treated microbial cell product of the microbe.

9. A DNA encoding a protein selected from the group consisting of (B), (D), (F), (H), (J), (L), (N), (P), (R), (T), (V), and (X), wherein said protein has an amino acid sequence defined as follows:
(B) an amino acid sequence including substitution, deletion, insertion, addition, and/or inversion of one or a plurality of amino acids in amino acid residue numbers 23 to 616 of SEQ ID NO: 6, and has at least 50% of the peptide-forming activity of a protein corresponding to unmutated amino acid residue numbers 23 to 616 of SEQ ID NO: 6 at50° C. and a pH of 8,
(D) an amino acid sequence including substitution, deletion, insertion, addition, and/or inversion of one or a plurality of amino acids in amino acid residue numbers 21 to 619 of SEQ ID NO: 12, and has at least 50% of the peptide-forming activity of a protein corresponding to unmutated amino acid residue numbers 21 to 619 of SEQ ID NO: 12 at 50° C. and a pH of 8,
(F) an amino acid sequence including substitution, deletion, insertion, addition, and/or inversion of one or a plurality of amino acids in amino acid residue numbers 23 to 625 of SEQ ID NO: 18, and has at least 50% of the peptide-forming activity of a protein corresponding to unmutated amino acid residue numbers 23 to 625 of SEQ ID NO: 18 at 50° C. and a pH of 8,
(H) an amino acid sequence including substitution, deletion, insertion, addition, and/or inversion of one or a plurality of amino acids in amino acid residue numbers 23 to 645 of SEQ ID NO: 23, and has at least 50% of the peptide-forming activity of a protein corresponding to unmutated amino acid residue numbers 23 to 645 of SEQ ID NO: 23 at 50° C. and a pH of 8,
(J) an amino acid sequence including substitution, deletion, insertion, addition, and/or inversion of one or a plurality of amino acids in amino acid residue numbers 26 to 620 of SEQ ID NO: 25, and has at least 50% of the peptide-forming activity of a protein corresponding to unmutated amino acid residue numbers 26 to 620 of SEQ ID NO: 25 at 50° C. and a pH of 8,
(L) an amino acid sequence including substitution, deletion, insertion, addition, and/or inversion of one or a plurality of amino acids in amino acid residue numbers 18 to 644 of SEQ ID NO: 27, and has at least 50% of the peptide-forming activity of a protein corresponding to unmutated amino acid residue numbers 18 to 644 of SEQ ID NO: 27 at 50° C. and a pH of 8,
(N) a mature protein region, having an amino acid sequence including substitution, deletion, insertion, addition, and/or inversion of one or a plurality of amino acids in an amino acid sequence consisting of SEQ ID NO: 6, and has at least 50% of the peptide-forming activity of a protein corresponding to unmutated SEQ ID NO: 6 at 50° C. and a pH of 8,
(P) a mature protein region, having an amino acid sequence including substitution, deletion, insertion, addition, and/or inversion of one or a plurality of amino acids in an amino acid sequence consisting of SEQ ID NO: 12, and has at least 50% of the peptide-forming activity of a protein corresponding to unmutated SEQ ID NO: 12 at 50° C. and a pH of 8,
(R) a mature protein region, having an amino acid sequence including substitution, deletion, insertion, addition, and/or inversion of one or a plurality of amino acids in an amino acid sequence consisting of SEQ ID NO: 18, and has at least 50% of the peptide-forming activity of a protein corresponding to unmutated SEQ ID NO: 18 at 50° C. and a pH of 8,
(T) a mature protein region, having an amino acid sequence including substitution, deletion, insertion, addition, and/or inversion of one or a plurality of amino acids in an amino acid sequence consisting of SEQ ID NO: 23, and has at least 50% of the peptide-forming activity of a protein corresponding to unmutated SEQ ID NO: 23 at 50° C. and a pH of 8,
(V) a mature protein region, having an amino acid sequence including substitution, deletion, insertion, addition, and/or inversion of one or a plurality of amino acids in an amino acid sequence consisting of SEQ ID NO: 25, and has at least 50% of the peptide-forming activity of a protein corresponding to unmutated SEQ ID NO: 25 at 50° C. and a pH of 8, or
(X) a mature protein region, having an amino acid sequence including substitution, deletion, insertion, addition, and/or inversion of one or a plurality of amino acids in an amino acid sequence consisting of SEQ ID NO: 27, and has at least 50% of the peptide-forming activity of a protein corresponding to unmutated SEQ ID NO: 27 at 50° C. and a pH of 8.

10. The DNA according to claim 9, wherein said plurality is 2 to 50 amino acid residues.

11. A recombinant DNA comprising the DNA according to claim 9.

12. A transformed cell comprising the recombinant DNA according to claim 11.

13. A method for producing a peptide-forming enzyme comprising:
culturing the transformed cell according to claim 12 in a medium for a time and under conditions suitable to produce the peptide-forming enzyme, and
accumulating the peptide-forming enzyme in the medium and/or transformed cell.

14. A method for producing a dipeptide comprising:
culturing the transformed cell according to claim 12 in a medium for a time and under conditions suitable to produce a peptide-forming enzyme in a culture, and
mixing the culture with a carboxy component and an amine component to synthesize a dipeptide by enzymatic catalysis facilitated by a peptide-forming enzyme encoded by said DNA.

15. The method for producing a dipeptide according to claim 14, wherein said cell is a microbe belonging to the genus Sphingobacterium that has an ability to form the dipeptide from the carboxy component and the amine component.

16. The method for producing a dipeptide according to claim 15, wherein said cell is separated from said culture.

17. The method for producing a dipeptide according to claim 15, wherein said cell is a treated microbial cell product of the microbe.

18. A DNA selected from the group consisting of (a), (c), (e), (g), (i), (k), (m), (o), (q), (s), (u), and (w), wherein said DNA has a base sequence defined as follows:
(a) a base sequence consisting of base numbers 127 to 1908 of SEQ ID NO: 5,
(c) a base sequence consisting of base numbers 121 to 1917 of SEQ ID NO: 11,
(e) a base sequence consisting of base numbers 127 to 1935 of SEQ ID NO: 17,
(g) a base sequence consisting of base numbers 127 to 1995 of SEQ ID NO: 22,
(i) a base sequence consisting of base numbers 104 to 1888 of SEQ ID NO: 24,
(k) a base sequence consisting of base numbers 112 to 1992 of SEQ ID NO: 26,
(m) a base sequence consisting of base numbers 61 to 1908 of SEQ ID NO: 5,
(o) a base sequence consisting of base numbers 61 to 1917 of SEQ ID NO: 11,
(q) a base sequence consisting of base numbers 61 to 1935 of SEQ ID NO: 17,
(s) a base sequence consisting of base numbers 61 to 1995 of SEQ ID NO: 22,
(u) a base sequence consisting of base numbers 29 to 1888 of SEQ ID NO: 24, or
(w) a base sequence consisting of base numbers 61 to 1992 of SEQ ID NO: 26.

19. A recombinant DNA comprising the DNA according to claim 18.

20. A transformed cell comprising the recombinant DNA according to claim 19.

21. A method for producing a peptide-forming enzyme comprising:
culturing the transformed cell according to claim 20 in a medium for a time and under conditions suitable to produce the peptide-forming enzyme, and
accumulating the peptide-forming enzyme in the medium and/or transformed cell.

22. A method for producing a dipeptide comprising:
culturing the transformed cell according to claim 20 in a medium for a time and under conditions suitable to produce a peptide-forming enzyme in a culture, and
mixing the culture with a carboxy component and an amine component to synthesize a dipeptide by enzymatic catalysis facilitated by a peptide-forming enzyme encoded by said DNA.

23. The method for producing a dipeptide according to claim 22, wherein said cell is a microbe belonging to the genus Sphingobacterium that has an ability to form the dipeptide from the carboxy component and the amine component.

24. The method for producing a dipeptide according to claim 23, wherein said cell is separated from said culture.

25. The method for producing a dipeptide according to claim 23, wherein said cell is a treated microbial cell product of the microbe.

26. A DNA selected from the group consisting of (b), (d), (f), (h), (j), (l), (n), (p), (r), (t), (v), and (x), wherein said DNA has a base sequence defined as follows:
(b) a base sequence that hybridizes under stringent conditions with a DNA having a base sequence complementary to a base sequence consisting of base numbers 127 to 1908 of SEQ ID NO: 5, and encodes a protein that has at least 50% of the peptide-forming activity at 50° C. and a pH of 8 of a protein encoded by unmutated base numbers 127 to 1908 of SEQ ID NO: 5,
(d) a base sequence that hybridizes under stringent conditions with a DNA having a base sequence complementary to a base sequence consisting of base numbers 121 to 1917 of SEQ ID NO: 11, and encodes a protein that has at least 50% of the peptide-forming activity at 50° C. and a pH of 8 of a protein encoded by unmutated base numbers 121 to 1917 of SEQ ID NO: 11,
(f) a base sequence that hybridizes under stringent conditions with a DNA having a base sequence complementary to a base sequence consisting of base numbers 127 to 1935 of SEQ ID NO: 17, and encodes a protein that has at least 50% of the peptide-forming activity at 50° C. and a pH of 8 of a protein encoded by unmutated base numbers 127 to 1935 of SEQ ID NO: 17,
(h) a base sequence that hybridizes under stringent conditions with a DNA having a base sequence complementary to a base sequence consisting of bases numbers 127 to 1995 of SEQ ID NO: 22, and encodes a protein that has at least 50% of the peptide-forming activity at 50° C. and a pH of 8 of a protein encoded by unmutated base numbers 127 to 1995 of SEQ ID NO: 22,
(j) a base sequence that hybridizes under stringent conditions with a DNA having a base sequence complementary to a base sequence consisting of base numbers 104 to 1888 of SEQ ID NO: 24, and encodes a protein that has at least 50% of the peptide-forming activity at 50° C. and a pH of 8 of a protein encoded by unmutated base numbers 104 to 1888 of SEQ ID NO: 24,
(l) a base sequence that hybridizes under stringent conditions with a DNA having a base sequence complementary to a base sequence consisting of base numbers 112 to 1992 of SEQ ID NO: 26, and encodes a protein that has at least 50% of the peptide-forming activity at 50° C. and a pH of 8 of a protein encoded by unmutated base numbers 112 to 1992 of SEQ ID NO: 26,
(n) a base sequence that hybridizes under stringent conditions with a DNA having a base sequence complementary to a base sequence consisting of base numbers 61 to 1908 of SEQ ID NO: 5, and encodes a protein containing a muture protein regaion and has at least 50% of the peptide-forming activity at 50° C. and a pH of 8 of a protein encoded by unmutated base numbers 61 to 1908 of SEQ ID NO: 5,
(p) a base sequence that hybridizes under stringent conditions with a DNA having a base sequence complementary to a base sequence consisting of base numbers 61 to 1917 of SEQ ID NO: 11, and encodes a protein containing a muture protein regaion and has at least 50% of the peptide-forming activity at 50° C. and a pH of 8 of a protein encoded by unmutated base numbers 61 to 1917 of SEQ ID NO: 11,
(r) a base sequence that hybridizes under stringent conditions with a DNA having a base sequence complementary to a base sequence consisting of base numbers 61 to 1935 of SEQ ID NO: 17, and encodes a protein containing a muture protein regaion and has at least 50% of the peptide-forming activity at 50° C. and a pH of 8 of a protein encoded by unmutated base numbers 61 to 1935 of SEQ ID NO: 17,
(t) a base sequence that hybridizes under stringent conditions with a DNA having a base sequence complementary to a base sequence consisting of base numbers 61 to 1995 of SEQ ID NO: 22, and encodes a protein containing a muture protein regaion and has at least 50% of the peptide-forming activity at 50° C. and a pH of 8 of a protein encoded by unmutated base numbers 61 to 1995 of SEQ ID NO: 22,
(v) a base sequence that hybridizes under stringent conditions with a DNA having a base sequence complementary to a base sequence consisting of base numbers 29 to 1888 of SEQ ID NO: 24, and encodes a protein containing a muture protein regaion and has at least 50% of the peptide-forming activity at 50° C. and a pH of 8 of a protein encoded by unmutated base numbers 29 to 1888 of SEQ ID NO: 24, or
(x) a base sequence that hybridizes under stringent conditions with a DNA having a base sequence complementary to a base sequence consisting of base numbers 61 to 1992 of SEQ ID NO: 26, and encodes a protein containing a muture protein regaion and has at least 50% of the peptide-forming activity at 50° C. and a pH of 8 of a protein encoded by unmutated base numbers 61 to 1992 of SEQ ID NO: 26.

27. A recombinant DNA comprising the DNA according to claim 26.

28. A transformed cell comprising the recombinant DNA according to claim 26.

29. A method for producing a peptide-forming enzyme comprising:
culturing the transformed cell according to claim 28 in a medium for a time and under conditions suitable to produce the peptide-forming enzyme, and
accumulating the peptide-forming enzyme in the medium and/or transformed cell.

30. A method for producing a dipeptide comprising:
culturing the transformed cell according to claim 28 in a medium for a time and under conditions suitable to produce a peptide-forming enzyme in a culture, and
mixing the culture with a carboxy component and an amine component to synthesize a dipeptide by enzymatic catalysis facilitated by a peptide-forming enzyme encoded by said DNA.

31. The method for producing a dipeptide according to claim 30, wherein said cell is a microbe belonging to the genus Sphingobacterium that has an ability to form the dipeptide from the carboxy component and the amine component.

32. The method for producing a dipeptide according to claim 31, wherein said cell is separated from said culture.

33. The method for producing a dipeptide according to claim 31, wherein said cell is a treated microbial cell product of the microbe.

34. The DNA according to claim 26, wherein stringent conditions are conditions under which washing is carried out at 60° C. at a salt concentration equivalent to 1×SSC and 0.1% SDS.

35. A recombinant DNA comprising the DNA according to claim 34.

36. A transformed cell comprising the recombinant DNA according to claim 35.

37. A method for producing a peptide-forming enzyme comprising:
culturing the transformed cell according to claim 36 in a medium for a time and under conditions suitable to produce the peptide-forming enzyme, and
accumulating the peptide-forming enzyme in the medium and/or transformed cell.

38. A method for producing a dipeptide comprising:
culturing the transformed cell according to claim 36 in a medium for a time and under conditions suitable to produce a peptide-forming enzyme in a culture, and
mixing the culture with a carboxy component and an amine component to synthesize a dipeptide by enzymatic catalysis facilitated by a peptide-forming enzyme encoded by said DNA.


SEQUENCE LISTING (27 sequences)

SEQ ID NO: 1 - protein, 9 aa, Empedobacter brevis
LFTAIYQPK

SEQ ID NO: 2 - protein, 9 aa, Empedobacter brevis
TNVTYTMPD

SEQ ID NO: 3 - DNA, 20 bp, Artificial Sequence
  Synthetic DNA
ttyacngcnathtaycarcc

SEQ ID NO: 4 - DNA, 23 bp, Artificial Sequence
  Synthetic DNA
tcnggcatngtrtangtnacrtt

SEQ ID NO: 5 - DNA, 2024 bp, Empedobacter brevis
  CDS (61)..(1908)
atttcttaataaaaactgaaatcttaatacatttatactatcgtaaaatttattgaacacgtgaaaaaattaacattaaaagtaactctacttacacttttgttgggaagtacagttggatttgcgcaagatgcaaaagcagattctgcttatgtgcgcgacaattacgaaaaaatagaacaagtaattccgatgcgcgatggtacaaagttatttacagctatttatcagccaaaagataaaacaaaacaatatcccgttttgttaaatcgtacgccttatacagttgcgccttatggtgtaaatgaatacaagaaatcgttaggaaattttcctacagaaatgcgcgaaggttttatttttgtttaccaagatgtgagaggaaaatggatgagcgaaggcgaatttgaagatgttcgacctataaatccttcaaaaagtaaaaaggcaattgacgaaagcacagatacatttgatacgctagaatggcttgctaaaaacttgaagaattacacgaaaaaagctggaatttatggaatttcgtatcctggtttttattcgacaatgagtttggttaattcgcatccaactctaaaagccgtttcgccacaagcgcccgttaccaattggtttttaggtgacgattttcatcataatggagttttattcttgaatgattctttctcatttatgactttttttggtgtaaaacgtccgcaaccaattacgccagataaaggtccgaaacgttttgaatatccaataaaagataattatagattttatgcaagtggctctgtaaaagagttgaaagataaatatttgcaagataatatcaagttttacaatgatttatttgcgcatccagattacgatcaattttggcaagatcgtaatgttttaccacatttaactaacgtgcaacctgctgtaatgacggttggaggtttttttgatgcagaagatgtctacggcgctttcgaaacgtataaagcaattgagaaacaaaatccgaaagcaacaaatattatggttgccggaccttggtttcatggtggttgggttcgtagcaacggaagtacttttggagatatgcaatttgcatcgaatacaagtgagcattatcagcaagaaatagaattgcctttttttaattattacttaaaagataaaggtaattttaaaccaaccgaagctacaatttttattacgggatctaacgaatggaaacaatttgatgcttggccaccaaaaaatgtaacaacacaaaaaatttatttgcaacaaaatggtaaaatagcttttaataaaaccaatacaacaactacttttgacgaatatgttgcagatccaaattctccagttccttattcaggaggagttttagaaactcgttcaagagaatatatggtcgatgatcaacgctttgcttctactcgtcctgatgttatggtgtatcaatctgatattttgacagaagatattacgcttgctggtcctgttatcaatcatttagtggtttctactacgggaacagacgctgattatgttgtaaaattgattgatgtttatcctgaaaacacgccaaaatttaataacaaattaatggctggatatcaaaatttgattcgtgcagaaattatgcgcggaaaatatagaaatagtttctctaaccccgaagctatggttccgaataaagaaacaaatgtaacgtacacgatgccagatgttggacatacatttaagaaaggacatcgcattatgattcaagttcagaacagttggtttcctttagcagatcgcaatccgcaacaatttatgaatgtttacgaagcaacttctaaagattatttaaaacaaacgcaacgaatttatcatacttcttatatcgaaattccggtattgaaataacaaaaaaatccagctaattagctggattttttttataatgttacttttcctatttttcctttatttccaactaaaattacatattttttatcgggcgaaaccgtacaagtatg

SEQ ID NO: 6 - protein, 616 aa, Empedobacter brevis
VKKLTLKVTLLTLLLGSTVGFAQDAKADSAYVRDNYEKIEQVIPMRDGTKLFTAIYQPKDKTKQYPVLLNRTPYTVAPYGVNEYKKSLGNFPTEMREGFIFVYQDVRGKWMSEGEFEDVRPINPSKSKKAIDESTDTFDTLEWLAKNLKNYTKKAGIYGISYPGFYSTMSLVNSHPTLKAVSPQAPVTNWFLGDDFHHNGVLFLNDSFSFMTFFGVKRPQPITPDKGPKRFEYPIKDNYRFYASGSVKELKDKYLQDNIKFYNDLFAHPDYDQFWQDRNVLPHLTNVQPAVMTVGGFFDAEDVYGAFETYKAIEKQNPKATNIMVAGPWFHGGWVRSNGSTFGDMQFASNTSEHYQQEIELPFFNYYLKDKGNFKPTEATIFITGSNEWKQFDAWPPKNVTTQKIYLQQNGKIAFNKTNTTTTFDEYVADPNSPVPYSGGVLETRSREYMVDDQRFASTRPDVMVYQSDILTEDITLAGPVINHLVVSTTGTDADYVVKLIDVYPENTPKFNNKLMAGYQNLIRAEIMRGKYRNSFSNPEAMVPNKETNVTYTMPDVGHTFKKGHRIMIQVQNSWFPLADRNPQQFMNVYEATSKDYLKQTQRIYHTSYIEIPVLK

SEQ ID NO: 7 - DNA, 40 bp, Artificial Sequence
  Synthetic DNA
gtatcacgaggccctagctgtggtgtcatggtcggtgatc

SEQ ID NO: 8 - DNA, 40 bp, Artificial Sequence
  Synthetic DNA
ttcggggattccatatgataccctttttacgtgaacttgc

SEQ ID NO: 9 - DNA, 38 bp, Artificial Sequence
  Synthetic DNA
gggaattccatatgaaaaaattaacattaaaagtaact

SEQ ID NO: 10 - DNA, 36 bp, Artificial Sequence
  Synthetic DNA
gggggctgcagtacttgtacggtttcgcccgataaa

SEQ ID NO: 11 - DNA, 1935 bp, Sphingobacterium sp.
  CDS (61)..(1917)
gaaaccaagtgtaaaattataatttacaccaaagaatgtactgaacaaataattatctgaatgaaaaatacaatttcgtgcctaactttagcgcttttaagcgcaagccagttacatgctcaaacagctgccgactcggcttatgttagagatcattatgaaaagaccgaagtagcaattcccatgcgagatgggaaaaaattatttactgcgatctacagtccaaaagacaaatccaagaaatatccagttttgctcaatagaacgccctacacggtttcaccttatgggcagaacgaatataaaaaaagcttgggaaactttccccaaatgatgcgtgaaggctatattttcgtttaccaggatgtccgtggcaagtggatgagcgaaggtgattttgaagatatacgtccgaccacgtacagcaaagataaaaaagcaatcgatgaaagtacggatacctatgatgcgcttgaatggttacagaaaaatctcaaaaactataatggcaaagccgggctctatgggatttcctatccaggcttctattctaccgtcggattggtcaaaacacacccgagcttgaaggcagtctccccacaggctcccgtaacagactggtatatcggcgacgacttccaccataatggcgtattgtttcttcaggatgcatttacattcatgtcaacctttggtgtccctcgtccaaaacccattacaccggatcaatttaagggcaaaattcagatcaaagaagccgataaatataacttttttgcagaagcaggaacagcgcgggaactcaaagaaaagtattttggtgactccgtacaattttggaatgacctgtttaagcatcccgactatgatgatttttggaaatcgcgtgtgatcacgaattctttacaggaggtaaaaccagctgtgatggtggttggtggtttctttgacgcggaagatgcttatggaacatttaagacctaccaatcgattgaggataaaagcaaaaaaaacaactcgattttagtcgcgggaccttggtatcatggcggttgggttcgtgcagaaggaaactatttaggtgatatccaatttgagaaaaaaaccagtattacttatcaggaacaatttgaacaaccatttttcaaatattacctaaaagatgaaggaaacttcgccccttccgaagctaacatttttgtttcaggcagcaacgaatggaaacatttcgaacagtggccaccaaaaaatgtagagacaaaaaaactatacttccaacctcaggggaaacttggatttgacaaagttcaacgtacagattcctgggatgaatatgtaacagaccctaataaacctgttccgcatcaaggtggggtaattcaaaaccgaacacgggagtatatggtagatgatcaacgtttcgcggctagtcgccctgatgtcatggtttatcaaacggaaccgttgacggaggacctgacgatagtaggcccaatcaaaaactttctcaaagtttcttcaacaggaacagacgcggactatgttgtcaaactgattgacgtttatccgaatgatgcagcaagttatcaaggaaaaacaatggctggatatcaaatgatggtacgtggtgagatcatggcggggaaataccgaaatggtttcgataaagcgcaggccttgactccaggtatggtcgaaaaggtgaattttgaaatgccagacgttgcgcataccttcaaaaaaggacatcgcattatggttcaggtacaaaactcatggtttccgctggcagaacgaaatccacaggtgtttttagcaccttatacagctaccaaagctgatttccgcaaagctacccaacgtatttttcacgatgtgaacaatgccacatacatcgaattttctgtcctcaaagattagcaggtaaattcgaaa

SEQ ID NO: 12 - protein, 619 aa, Sphingobacterium sp.
MKNTISCLTLALLSASQLHAQTAADSAYVRDHYEKTEVAIPMRDGKKLFTAIYSPKDKSKKYPVLLNRTPYTVSPYGQNEYKKSLGNFPQMMREGYIFVYQDVRGKWMSEGDFEDIRPTTYSKDKKAIDESTDTYDALEWLQKNLKNYNGKAGLYGISYPGFYSTVGLVKTHPSLKAVSPQAPVTDWYIGDDFHHNGVLFLQDAFTFMSTFGVPRPKPITPDQFKGKIQIKEADKYNFFAEAGTARELKEKYFGDSVQFWNDLFKHPDYDDFWKSRVITNSLQEVKPAVMVVGGFFDAEDAYGTFKTYQSIEDKSKKNNSILVAGPWYHGGWVRAEGNYLGDIQFEKKTSITYQEQFEQPFFKYYLKDEGNFAPSEANIFVSGSNEWKHFEQWPPKNVETKKLYFQPQGKLGFDKVQRTDSWDEYVTDPNKPVPHQGGVIQNRTREYMVDDQRFAASRPDVMVYQTEPLTEDLTIVGPIKNFLKVSSTGTDADYVVKLIDVYPNDAASYQGKTMAGYQMMVRGEIMAGKYRNGFDKAQALTPGMVEKVNFEMPDVAHTFKKGHRIMVQVQNSWFPLAERNPQVFLAPYTATKADFRKATQRIFHDVNNATYIEFSVLKD

SEQ ID NO: 13 - DNA, 30 bp, Artificial Sequence
  Synthetic DNA
gggaattccatatgaaaaatacaatttcgt

SEQ ID NO: 14 - DNA, 29 bp, Artificial Sequence
  Synthetic DNA
gctctagactaatctttgaggacagaaaa

SEQ ID NO: 15 - DNA, 17 bp, Artificial Sequence
  Synthetic DNA
gaygayttycaycayaa

SEQ ID NO: 16 - DNA, 20 bp, Artificial Sequence
  Synthetic DNA
tgrtcrtcnaccatrtaytc

SEQ ID NO: 17 - DNA, 1974 bp, Pedobacter heparinus
  CDS (61)..(1935)
aaacctatcccgtattcagcaatcaattccatatatttatccttaaaaaaaccttcctctatgactcctttcaaatcgttctccttcatttttctctttatttttaccagtctttctgcttctgcacaacagtccgactctgcttatatacgtcagaactataccaaaatagaaaggctgatccctatgcgggatggcattaagctatttacagccatttacatccccaaagacaaaagcaagaagtatccttttatgctcaaccgtactccttataccgtttcgccttatggcgaaaacaattataaaacaagccttggcccctctccgctctttataaaagaaggctttatctttgtttatcaggatgtaaggggcaaatggatgagtgagggaaaatttgaagacgtaaggccgcaaatagccagcaagaaacgcaaaacggatattgatgaaagctccgatacttatgatacgatcgactggctgatcaggaacattcctggaaacaaccgtaaaaccggtatttacggtatctcatacccaggcttttatgctactgctgccctaccagatgcgcatccatctttaaaggcagtatcgccccaggctccggttaccgactggtttataggcgatgattttcatcacaatggcaccttgttccttgcagatatctttagcttctattataccttcggggtaccgcgacctcaaccaattacgcccgacaaacgtccaaaaccctttgatttcccggttaaagacaactaccgtttttttcttgaactgggccccttaaaaaacatcaccaaaaaatattatggcgataccatacgattctggaatgatatcaatgcgcataccaattatgatgccttctggaaagcccgtaacattacgccgcatttaattggtgtaaaacctgcagttttggtagttggcggcttctttgatgcagaagacctttacggtacgcttaaaacctatcaggccatcgaaaaacaaaatccatcctcaaaaaacaacctcgttatgggcccctggtaccatggtggctgggcaagaagtacgggaagcagtttcggggatattaatttcggacagccaaccagtacttcataccagcaaaatgttgagttccctttctttatgcaatacctcaaagaggcaccggatgcaaaaattgcagaggcaaccatttttatcactggcagcaatgaatggaagaaatttagctcctggccacctcaggatacagaagaaagaacattatacctgcagcccaatggcaaactgagctttgagaaggtacagcggaccgacagctgggatgaatatgtaagtgatcccaattcacctgtcccttatcaggatggcatacaaaccagcagaacccgggaatatatgatcgatgaccagcgttttgcctcgcgcagaccggatgtaagggtattccaaacagagcccctcagttccgaccttacacttaccggcccggtattggccaaactggtggtatcaaccacaggtacggatgcagattatgtggtaaaactgatagatgtatatccggaagatacaccaaatcctgtacctaaccctaaaaacctgatcatgggtggttaccagatgctggtacgcggcgagatcatgcgtggaaaataccgtaatagctttgaaaaacccgagccttttgttcctggaacaattacaaaagtaaactatgcccttccggatgtagcccatacctttaaaaaaggccaccgcatcatgatccaggtccagaattcatggtttcccctggccgaccggaatccacagcagtttatggacatttaccaggccgaacctggcgatttcagaaaagctacgcataggatcttccacgatgtacacaatgcatctgcaattacggtaaacgtactgaaaccttaaaacggatgaaaccagtatattgtgccatccttactt

SEQ ID NO: 18 - protein, 625 aa, Pedobacter heparinus
MTPFKSFSFIFLFIFTSLSASAQQSDSAYIRQNYTKIERLIPMRDGIKLFTAIYIPKDKSKKYPFMLNRTPYTVSPYGENNYKTSLGPSPLFIKEGFIFVYQDVRGKWMSEGKFEDVRPQIASKKRKTDIDESSDTYDTIDWLIRNIPGNNRKTGIYGISYPGFYATAALPDAHPSLKAVSPQAPVTDWFIGDDFHHNGTLFLADIFSFYYTFGVPRPQPITPDKRPKPFDFPVKDNYRFFLELGPLKNITKKYYGDTIRFWNDINAHTNYDAFWKARNITPHLIGVKPAVLVVGGFFDAEDLYGTLKTYQAIEKQNPSSKNNLVMGPWYHGGWARSTGSSFGDINFGQPTSTSYQQNVEFPFFMQYLKEAPDAKIAEATIFITGSNEWKKFSSWPPQDTEERTLYLQPNGKLSFEKVQRTDSWDEYVSDPNSPVPYQDGIQTSRTREYMIDDQRFASRRPDVRVFQTEPLSSDLTLTGPVLAKLVVSTTGTDADYVVKLIDVYPEDTPNPVPNPKNLIMGGYQMLVRGEIMRGKYRNSFEKPEPFVPGTITKVNYALPDVAHTFKKGHRIMIQVQNSWFPLADRNPQQFMDIYQAEPGDFRKATHRIFHDVHNASAITVNVLKP

SEQ ID NO: 19 - DNA, 38 bp, Artificial Sequence
  Synthetic DNA
gggaattccatatgactcctttcaaatcgttctccttc

SEQ ID NO: 20 - DNA, 30 bp, Artificial Sequence
  Synthetic DNA
cccaagcttttaaggtttcagtacgtttac

SEQ ID NO: 21 - DNA, 17 bp, Artificial Sequence
  Synthetic DNA
athttygtntaycarga

SEQ ID NO: 22 - DNA, 2018 bp, Taxeobacter gelupurpurascens
  CDS (61)..(1995)
ctgaatgtctgctgacgaattggaactacattaggctcgttcttcacctacccttccactatgccctactctttcccgaaagttgccgccctgagtggcctactggtggccggtttatccggtgcccacgcccaaactcctgttacctatccgctggcttctgaggctgaaaaagcgcagctggcggtggtactagccgatacggcttacatcaaggagcgctataccaaaacagaatatcagattccgatgcgcgatggggtgaagttgtacaccattgtgtacgcgcccaacgatgccaacaaggtaaagtaccctattctgctcaaccgtaccccttacgctattggcccctacggccccggcaaatacaagctcaacctgggccccagcagcacgatgatgcatgagggatacatcttcgcctaccaggatgtgcgtgggcgatatatgtcggaaggagagtttgtggatgtgcgccccgaaaaggacatgcacaaaggcaagaacgacatcgatgaaggcaccgacacctacgataccattgagtggcttctgaagcacgggcccaagaataacggccgcgtaggccagtggggcatctcctaccccggctactataccgctactggcctactgagccgccacaaggccctaaaggcatcctcaccgcaggcccctattgccgactggttctgggacgattttcaccacaacggcgcgttcttcctgccgcacgctttcaacttcctggcctcctttgggctggcccgcccccagcccacgcctaccggcaaccccggcttcaagcacggcacccccgatggctacgattttttcctgaagatgggtccgctgaaaaacgctgatgccaactactacaaaggcaaagtggccttctggaacgaaatggccagccaccccaactacgacgaattctggcaggcccgtaacctacgcccccacctcaagaacctcaacaaaggcaccgcggtgctcacggttggtggcttcaatgatgccgaggacctgtttggcgccctgaaaacctacgaaagcatcgagaagcaaaaccccggcatgcgcaacggcctcgtgatggggccgtgggtacacggtggctgggcccgcggcactggcgaaatggtaggcaatgtggcctacggcgagtcgccgtcgttgtattaccagaagcagattgaagcgccgttcttcaaatcatatctgaaggatggcaaacctgccgctacccccgaggctaccatctttgaaagcggcaccaaccgctggcgcagcttcgaaacctggccgcccaaagaagccaaagagcgcactttgtactttcagtcggccgggaaaatcggcttcgagaagcctgccagtggcctagagtacgaccagttcctcagcgacccggctcacccagtgcctttcaccgaagctacggctacgggcatgacccgcgagtacatgaccgacgaccagcgcttcgccagccgccgccccgacgtgctgacctaccagaccgaagcgcttaccgaggacatgacgctggctggccctatcgaggcgctgttgcaggtagccaccaccggcaccgatgccgactgggtagtgaagattattgatgtgtaccccgacgatacgcccaacaaccccagcacgaaccccgccgtgaaactgggcggctaccagcagatggttcgctccgaggtgatgcgcggtcgtttccgcaacagcttctccaagcccgaagcctttgtaccggaacaggtaacggccgtgcccttcacggtgcaggacctgtgccacaccttccggaaaggacaccgcctgatggtgcaggtgcaaagcagctggttcccgattgttgaccgcaacccgcagaccttcgtacccaatattttcgaggccgatgagaaggatttccaggccgccacgcatcggctgtaccattcgccggcgcatagctcgcagctcacgttgcgcgttctgtaggccactctaaacaggctcgg

SEQ ID NO: 23 - protein, 645 aa, Taxeobacter gelupurpurascens
MPYSFPKVAALSGLLVAGLSGAHAQTPVTYPLASEAEKAQLAVVLADTAYIKERYTKTEYQIPMRDGVKLYTIVYAPNDANKVKYPILLNRTPYAIGPYGPGKYKLNLGPSSTMMHEGYIFAYQDVRGRYMSEGEFVDVRPEKDMHKGKNDIDEGTDTYDTIEWLLKHGPKNNGRVGQWGISYPGYYTATGLLSRHKALKASSPQAPIADWFWDDFHHNGAFFLPHAFNFLASFGLARPQPTPTGNPGFKHGTPDGYDFFLKMGPLKNADANYYKGKVAFWNEMASHPNYDEFWQARNLRPHLKNLNKGTAVLTVGGFNDAEDLFGALKTYESIEKQNPGMRNGLVMGPWVHGGWARGTGEMVGNVAYGESPSLYYQKQIEAPFFKSYLKDGKPAATPEATIFESGTNRWRSFETWPPKEAKERTLYFQSAGKIGFEKPASGLEYDQFLSDPAHPVPFTEATATGMTREYMTDDQRFASRRPDVLTYQTEALTEDMTLAGPIEALLQVATTGTDADWVVKIIDVYPDDTPNNPSTNPAVKLGGYQQMVRSEVMRGRFRNSFSKPEAFVPEQVTAVPFTVQDLCHTFRKGHRLMVQVQSSWFPIVDRNPQTFVPNIFEADEKDFQAATHRLYHSPAHSSQLTLRVL

SEQ ID NO: 24 - DNA, 1931 bp, Cyclobacterium marinum
  CDS (29)..(1888)
cccaaagcattaacaaaataatttagtcatgaaacactgttacaaacttctggtcttttacacattatttttgatgaccacaaactgggctttatcacaagccattaatggatatgataaggcagcctatgacattcctatgcgagatggagttcaccttcacaccatcgtctatagccccaaagatttatcgcagccctatcctatattgatgcaaaggacaccttacagcgccggcccttatggtcctggaaatatgaaaaataagcttggcccttctcagtttttaatgaacgatggctatatatttgtttaccaggatgtaagagggcggtggatgtcggaaggatcctatgacaacatgcgccctaccctatccaaatcagaaagaaattccaaccaaatagacgaaagcacagacacctatgataccatagaatggttgctcgccaatatcaaaaatcacaatgaaaaagtaggcctatggggaatcagctatcccggattttatagtgctgcagcccttccttttgcccatccaaacctgaaagccgtttcccctcaagcacccataggggatttttactttgatgattttcatcataacggtgcttacttattaagttattggttggccacttctgttttcggctaccaaaaagacggccctacacaggaagcatggtatggcatggtgaatccggaaacaaatgacggctatcagttttttatggatatggggccattaaaaaatgccgataaatggtatggtgaagacaattttttctggcaacaacttaaaaacaatcctgattacaacgctttctggcaaaagagaagtattattcctcacttaaaagaagtgaagcctgcagttttaaccgttgggggctggtttgatgcagaagatctctatggaccacttacaatttataaaaccattgaaaaaaataatcctgagacctacaataccattgtcatgggcccttggtcccacggagattggtcaagggaacctggatcacaggtcatttcaaatatttattttggtgattctatctccacatggtatcaaaaaaatatagaacgtgtttttttcaatcattttctaaaagaatccgaaaatagcaatcctgcccttcctgaagcctacatgtttgataccggaaaacataaatgggaaaaatttgacgattggcctcctaaagaaagccaatggaaaagcttttactttcaagagaaaggagagttaactgaggtaacacctgagggaaataggtttactacctatgtctcagacccctctaatcctgtcccctatagtcaagatattaaactaaacttcactccgagaaaatacatggccgatgaccagcgatttgcagccagaagaccggacgtactgacctttacgagcgaagtattaagtcaagacatgacgcttgcgggggaagtcatggcaaacttaaaagttgccacttcacaaactgatgctgattgggtagttaaaatcatcgatatatttcccggagatcagccaaatcatgcctatgttttagatggggtggacatgggcaattaccacctaatggttcgttcagaggtaattagagggaggtatagagaaagttttgagtttcctaaaccctttgttcctgatcaaatcactgctgttgatttcaggttacaagatcttttccatactttcaaaaaggggcataaaattcaaatacaaatacaaagtacttggtttcccctaattgatcgaaatccccaaaaatatgtacaaaacatatttgaagctgaggaagccgattttgtcaaagccacccatagggtttttcatacagaaaagtttgccagcaaaattgaagtaatggttcttccttagaattagaatggtttaaaattactatttgtagcagaagata

SEQ ID NO: 25 - protein, 620 aa, Cyclobacterium marinum
MKHCYKLLVFYTLFLMTTNWALSQAINGYDKAAYDIPMRDGVHLHTIVYSPKDLSQPYPILMQRTPYSAGPYGPGNMKNKLGPSQFLMNDGYIFVYQDVRGRWMSEGSYDNMRPTLSKSERNSNQIDESTDTYDTIEWLLANIKNHNEKVGLWGISYPGFYSAAALPFAHPNLKAVSPQAPIGDFYFDDFHHNGAYLLSYWLATSVFGYQKDGPTQEAWYGMVNPETNDGYQFFMDMGPLKNADKWYGEDNFFWQQLKNNPDYNAFWQKRSIIPHLKEVKPAVLTVGGWFDAEDLYGPLTIYKTIEKNNPETYNTIVMGPWSHGDWSREPGSQVISNIYFGDSISTWYQKNIERVFFNHFLKESENSNPALPEAYMFDTGKHKWEKFDDWPPKESQWKSFYFQEKGELTEVTPEGNRFTTYVSDPSNPVPYSQDIKLNFTPRKYMADDQRFAARRPDVLTFTSEVLSQDMTLAGEVMANLKVATSQTDADWVVKIIDIFPGDQPNHAYVLDGVDMGNYHLMVRSEVIRGRYRESFEFPKPFVPDQITAVDFRLQDLFHTFKKGHKIQIQIQSTWFPLIDRNPQKYVQNIFEAEEADFVKATHRVFHTEKFASKIEVMVLP

SEQ ID NO: 26 - DNA, 2036 bp, Psycloserpens burtonensis
  CDS (61)..(1992)
catattcgtaaaatagctataagtttttgtaaatttagtcaatcaaaattttaaatgtaaatgaagactctttttaaattgttgctcctatttgtatttgttctaacgtcttgtaataaggccaacaaagacgctactgaaattgtgaaaaccgaagtagaagatacttacgttaaagataattataacaaacaagaggtgactattgaaatgcgcgatggtataaaacttcacacgaccatttattcaccaaaagatgaaagtcagacctatcctattttaatgatgagaacaccatatagttctcaaccttatggtgacaatgagtttaagacgaaaattggtcctaatgttcatttaatgaaagaagggaatattgttgtgtatcaagatgtacgaggtcgttggatgagtgaaggtgtctatgataatatgcgtgcttatatcccaaataaaacagaggattctcaaattgatgaggcatcagacacttatgacacgattgactggctggtaaataacgtagaaaataataacgggaatgttggtacttggggaatttcatatcctggtttttatgctacatattctactatagacgcacacccagctttaaaagcagcatcgcctcaagcgtgtattggagatttcttttttgacgattttcatcataatggtgcttttttattaagttattttagagcagtgtctttatttggtacgacaaaagataaacctacagattctgcttggtataagtttccagaaatgaaaacacaagatcaatatcaattttttcttgatgctggacctttaagtaatttgaacaagtatttccaatatgacacaccagacgacacatctgtatccaagtctgataggatagatgatgtgttttggaaagaaattgtagagcatccaaactacgatacgatatggaaatctaaaggtttaattcaaaacctaaaagatattaagccaagtgtagcgacaatgattgtgggagggttatttgatgccgaagatttatatgggccatttgaaacttataaaacgatagaaaaacataatcctgataattataatattatggtttttgggccttgggatcatggtcgttgggctaggagtgacgttaaaaattatgttggaaattatttcttcggagattctatatctctaaaatttcaacgtgatgttgaaacgaagttttttaatcattttttaaaaggaaaaggcgacaagaactcagggttaccagaagcatatgtatttgattctggtaaaaaggaatggagtagctttgacagctggcctccaaagcaagcagaaaaacaagccatgtatcttaatgccaaccaagagctatcagattcaaaaaaaggaaatactagtgagacatttgttagtgatttaaaacgccctgtaccttattccgaagatattaaaacagttttcacaccacgaaaatacatgacagacgatcagcgttttgcagcacgacgtcctgatgttcttatatttgagaccgatattcttgaggaagatataaccttagctggtgatattttagcgcagcttaatgtgtcaactacagggacagatgcagattggattgtcaaaatagtagatgttcatccagcagatgctgaggagcaaaaagaaggtatgcaagaccatttatcaatgagtaattatcatttgatggtgaggagtgaagtgatgcgcggtcgttttagaaatagttttgaaaacccagagccatttgtgccaaaccaaccaacagatgtcaatatcaagttacaagatgtacatcatacatttaaaaaaggtcacaaattacaagtgcaagttcagagtacgtggtttccacttattgatttgaacccgcaaacatttgtgcctaatatttataaagcaaaagaaagcgattttaaaacccaaacacattcggtttttaacgattctaaaattgagtttacggttttgaaataagagtagatgactaaatttgccaaggtagatttagtcttttt

SEQ ID NO: 27 - protein, 644 aa, Psycloserpens burtonensis
MKTLFKLLLLFVFVLTSCNKANKDATEIVKTEVEDTYVKDNYNKQEVTIEMRDGIKLHTTIYSPKDESQTYPILMMRTPYSSQPYGDNEFKTKIGPNVHLMKEGNIVVYQDVRGRWMSEGVYDNMRAYIPNKTEDSQIDEASDTYDTIDWLVNNVENNNGNVGTWGISYPGFYATYSTIDAHPALKAASPQACIGDFFFDDFHHNGAFLLSYFRAVSLFGTTKDKPTDSAWYKFPEMKTQDQYQFFLDAGPLSNLNKYFQYDTPDDTSVSKSDRIDDVFWKEIVEHPNYDTIWKSKGLIQNLKDIKPSVATMIVGGLFDAEDLYGPFETYKTIEKHNPDNYNIMVFGPWDHGRWARSDVKNYVGNYFFGDSISLKFQRDVETKFFNHFLKGKGDKNSGLPEAYVFDSGKKEWSSFDSWPPKQAEKQAMYLNANQELSDSKKGNTSETFVSDLKRPVPYSEDIKTVFTPRKYMTDDQRFAARRPDVLIFETDILEEDITLAGDILAQLNVSTTGTDADWIVKIVDVHPADAEEQKEGMQDHLSMSNYHLMVRSEVMRGRFRNSFENPEPFVPNQPTDVNIKLQDVHHTFKKGHKLQVQVQSTWFPLIDLNPQTFVPNIYKAKESDFKTQTHSVFNDSKIEFTVLK